(19) United States
(12) Patent Application Publication
(10) Pub. No.: US 2024/0153289 A1
(43) Pub. Date: May 9, 2024

(54) SYSTEMS AND METHODS FOR CELL ANALYSIS

(71) Applicant: DEEPCELL, INC., Menlo Park, CA (US)

(72) Inventors: Mahdokht Masaeli, Menlo Park, CA (US); Mahyar Salek, Menlo Park, CA (US)

(73) Assignee: DEEPCELL, INC., Menlo Park, CA (US)

(21) Appl. No.: 18/546,260

(22) PCT Filed: Feb. 17, 2022
(86) PCT No.: PCT/US2022/016748

§ 371 Date: Aug. 11, 2023

Related U.S. Application Data
(60) Provisional application No. 63/151,394, filed on Feb. 19, 2021.
(60) Provisional application No. 63/174,182, filed on Apr. 13, 2021.

(51) Int. Cl. G06V 20/69; G06V 10/762; G06V 10/82; G16B 15/00; G16B 20/00

(52) U.S. Cl. CPC G06V 20/698; G06V 10/762; G06V 10/82; G06V 20/695; G16B 15/00; G16B 20/00

(57) ABSTRACT

The present disclosure provides systems and methods for classifying and sorting a cell. The method can comprise processing image data of cells to generate a cell morphology map comprising a plurality of morphologically-distinct clusters corresponding to different types or states of the cells. The method can comprise using a classifier to automatically classify a cellular image sample based on its proximity, correlation, or commonality with one or more of the morphologically-distinct clusters.

### CROSS-REFERENCE

This application claims the benefit of U.S. Provisional Patent Application No. 63/151,394, filed Feb. 19, 2021, and U.S. Provisional Patent Application No. 63/174,182, filed Apr. 13, 2021, each of which is entirely incorporated herein by reference.

### BACKGROUND

Analysis of a cell (e.g., determination of a type or a state of the cell) can be accomplished by examining, for example, one or more images of the cell that is tagged (e.g., stained with a polypeptide, such as an antibody, against a target protein of interest within the cell; with a polynucleotide against a target gene of interest within the cell; with probes to analyze gene expression profile of the cell via polymerase chain reaction; or with a small molecule substrate that is modified by the target protein) or sequencing data of the cell (e.g., gene fragment analysis, whole-genome sequencing, whole-exome sequencing, RNA-seq, etc.). Such methods can be used to identify cell type (e.g., stem cell or differentiated cell) or cell state (e.g., healthy or disease state). Such methods can require treatment of the cell (e.g., antibody staining, cell lysis or sequencing, etc.) that can be time-consuming and/or costly.

### SUMMARY

In view of the foregoing, recognized herein is a need for alternative methods and systems for analyzing cells (e.g., previously uncharacterized or unknown cells). For example, recognized herein is a need for method for analyzing cells without pretreatment of the cells to, e.g., tag a target protein or gene of interest in the cells, obtain sequencing data of the cells, etc.

Accordingly, in some embodiments, the present disclosure provides methods and systems for analyzing (e.g., automatically classifying) cells based on one or more morphological features of the cells. In some embodiments, the present disclosure provides methods and systems for sorting the cells into a plurality of sub-populations based on the one or more morphological features of the cells. In some embodiments, the present disclosure provides a reference database (e.g., a library, an atlas, etc.) of annotated images of different cells that can be used to analyze one or more news images of cells, e.g., based on one or more morphological features of the cells extracted from the one or more new images.

An aspect of the present disclosure provides a method comprising: (a) obtaining image data of a plurality of cells, wherein the image data comprises tag-free images of single cells; (b) processing the image data to generate a cell morphology map, wherein the cell morphology map comprises a plurality of morphologically-distinct clusters corresponding to different types or states of the cells; (c) training a classifier using the cell morphology map; and (d) using the classifier to automatically classify a cellular image sample based on its proximity, correlation, or commonality with one or more of the morphologically-distinct clusters.

Another aspect of the present disclosure provides a method comprising: (a) processing a sample and obtaining cellular image data of the sample; (b) processing the cellular image data to identify one or more morphological features that are potentially of interest to a user; and (c) displaying, on a graphical user interface (GUI), a visualization of patterns or profiles associated with the one or more morphological features.

Another aspect of the present disclosure provides a cell analysis platform comprising: a cell morphology atlas (CMA) comprising a database having a plurality of annotated single cell images that are grouped into morphologically-distinct clusters corresponding to a plurality of predefined cell classes; a modeling library comprising a plurality of models that are trained and validated using datasets from the CMA, to identify different cell types and/or states based at least on morphological features; and an analysis module comprising a classifier that uses one or more of the models from the modeling library to (1) classify one or more images taken from a sample and/or (2) assess a quality or state of the sample based on the one or more images.

Another aspect of the present disclosure provides a method comprising: (a) obtaining image data of a plurality of cells, wherein the image data comprises images of single cells captured using a plurality of different imaging modalities; (b) training a model using the image data; and (c) using the model with aid of a focusing tool to automatically adjust in real-time a spatial location of one or more of cells in a sample within a flow channel as the sample is being processed.

Another aspect of the present disclosure provides a method comprising: (a) obtaining image data of a plurality of cells, wherein the image data comprises images of single cells captured under a range of focal conditions; (b) training a model using the image data; (c) using the model to assess a focus of one or more images of one or more of cells in a sample within a flow channel as the sample is being processed; and (d) automatically adjusting in real-time an imaging focal plane based on the image focus assessed by the model.

Another aspect of the present disclosure provides a method comprising: (a) obtaining image data of a plurality of cells, wherein the image data comprises images of single cells captured using a plurality of different imaging modalities; (b) training an image processing tool using the image data; and (c) using the image processing tool to automatically identify, account for, and/or exclude artifacts from one or more images of one or more cells in a sample as the sample is being processed.

Another aspect of the present disclosure provides an online crowdsourcing platform comprising: a database storing a plurality of single cell images that are grouped into morphologically-distinct clusters corresponding to a plurality of predefined cell classes; a modeling library comprising one or more models; and a web portal for a community of users, wherein the web portal comprises a graphical user interface (GUI) that allows the users to (1) upload, download, search, curate, annotate, or edit one or more existing images or new images into the database, (2) train or validate the one or more models using datasets from the database, and/or (3) upload new models into the modeling library.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides a method of identifying a disease cause in a subject, the method comprising (a) obtaining a biological sample from the subject; (b) suspending the sample into a carrier, to effect constituents of the biological sample to (i) flow in a single line and (ii) rotate relative to the carrier; (c) sorting the constituents into at least two populations based on at least one morphological characteristic that is identified substantially concurrently with the sorting of the constituents; and (d) determining a disease cause of the subject as indicated by at least one population of the at least two populations.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications, and NCBI accession numbers mentioned in this specification are herein incorporated by reference to the same extent as if each individual publication, patent, patent application, or NCBI accession number was specifically and individually indicated to be incorporated by reference. To the extent publications and patents, patent applications, or NCBI accession numbers incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 schematically illustrates an example method for classifying a cell.

FIG. 2 schematically illustrates different methods of representing analysis data of image data of cells.

FIG. 3 schematically illustrates different representations of analysis of image data of a population of cells.

FIG. 4 schematically illustrates a method for a user to interact with a method for analyzing image data of cells.

FIG. 5 schematically illustrates a cell analysis platform for analyzing image data of one or more cells.

FIG. 6 schematically illustrates an example microfluidic system for sorting one or more cells.

FIG. 7 shows a computer system that is programmed or otherwise configured to implement methods provided herein.

FIGS. 8a-8f schematically illustrate an example system for classifying and sorting one or more cells.

FIGS. 9a-9e show a depiction of the model training, analysis, and sorting modes.

FIGS. 10a-10m show performance of the convolutional neural network (CNN) cell classifier as disclosed herein.

FIGS. 11a-d show example cell morphology plotting and analysis.

FIGS. 12a-12e show an additional example cell morphology plotting and analysis.

FIG. 13 demonstrates application of integrated gradients approach on an non-small-cell lung carcinomas (NSCLC) adenocarcinoma cell demonstrating pixels that supports inferring it as NSCLC in addition to pixels that oppose inferring it as other cell types.

FIGS. 14a and 14b illustrates results of random sorting of cells.

FIG. 15 shows the proportion of frame-shift mutation c.572_572delC in the TP53 gene in controlled mixtures before and after enrichment. The cell lines H522 and A549 are homozygous and wildtype respectively for this frame-shift mutation.

FIG. 16 shows accuracy of single nucleotide polymorphisms (SNP)-based mixture fraction estimates in control DNA mixtures. Each composite sample contained 250 pg of bulk DNA drawn from two individuals and the mixture proportion of DNA from the second individual was set at 5%, 10%, 20%, 30%, 40%, 60%, 80% and 90%. A close correspondence was found between the known and estimated mixture proportions.

FIG. 17 shows determination of purity of A549 cells enriched using the sorting platform as disclosed herein, from a 40 cells/ml spike-in into whole blood. The purity and blood sample genotypes were estimated with an expectation-maximization (EM) algorithm. Green triangles, blue diamonds and red circles denote AA, AB and BB genotypes respectively in the blood sample used as a base for the spike-in mixture; dotted lines represent the expected allele fractions for the three blood genotypes at the inferred purity of 43%, which is also the slope of the lines.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular.

I. Overview

One or more morphological properties of a cell can be used to, for example, study cell type and cell state, or to diagnose diseases. In some cases, cell shape can be one of the markers of cell cycle. Eukaryotic cells can show physical changes in shape which can be cell-cycle dependent, such as a yeast cell undergoing budding or fission. In some cases, cell shape can be an indicator of cell state and, thus, can be an indicator used for clinical diagnostics. In some cases, shape of a blood cell may change due to many clinical conditions, diseases, and medications (e.g., changes in red blood cells' morphologies resulting from parasitic infections). Additional examples of the morphological properties of the cell that can be used to analyze the cell can include, but are not limited to, features of cell membrane, nuclear-to-cytoplasm ratio, nuclear envelope morphology, and chromatin structure Methods, systems, and databases provided herein can be used analyze cells (e.g., previously uncharacterized or unknown cells) based on (e.g., solely on) such morphological properties of the cells.

Analyzing a cell based on one or more images of the cell and one or more morphological features of the cells extracted thereform—without the need to rely on other utilized methods of analyzing cells (e.g., identifying) cells (e.g., DNA analysis or genomics, RNA analysis or transcriptomics, protein analysis or proteomics, metabolite analysis or metabolomics, etc.)—can enhance speed and/or scalability of cell analysis systems and methods while maintaining or even enhancing accuracy of the analysis. In some cases, Analysis of a population of cells based on their morphological features can uncover unique or new parameters to define a cell or a collection of cells (e.g., clusters of cells) that would otherwise not be identified in other methods.

II. Methods and Platforms for Cell Analysis

The present disclosure describes various methods, e.g., a method for analyzing or classifying a cell, and platforms usable for or capable of performing such methods. The method can comprise obtaining image data of a plurality of cells, wherein the image data comprises tag-free images of single cells. The method can further comprise processing the image data to generate a cell morphology map (e.g., one or more cell morphology maps). The cell morphology map can comprise a plurality of morphologically-distinct clusters corresponding to different types or states of the cells. The method can further comprise training a classifier (e.g., a cell clustering machine learning algorithm or deep learning algorithm) using the cell morphology map. In some the classifier can be configured to classify (e.g., automatically classify) a cellular image sample based on its proximity, correlation, or commonality with one or more of the morphologically-distinct clusters. Thus, in some cases, the method can further comprise using the classifier to classify (e.g., automatically classify) the cellular image sample accordingly.

The term “morphology” of a cell as used herein generally refers to the form, structure, and/or configuration of the cell. The morphology of a cell can comprise one or more aspects of a cell's appearance, such as, for example, shape, size, arrangement, form, structure, pattern(s) of one or more internal and/or external parts of the cell, or shade (e.g., color, greyscale, etc.). Non-limiting examples of a shape of a cell can include, but are not limited to, circular, elliptic, shmoo-like, dumbbell, star-like, flat, scale-like, columnar, invaginated, having one or more concavely formed walls, having one or more convexly formed walls, prolongated, having appendices, having cilia, having angle(s), having corner(s), etc. A morphological feature of a cell may be visible with treatment of a cell (e.g., small molecule or antibody staining). Alternatively, the morphological feature of the cell may not and need not require any treatment to be visualized in an image or video.

The term “tag” as used herein generally refers to a heterologous composition detectable by fluorescence, spectroscopic, photochemical, biochemical, immunochemical, electrical, optical, chemical, or other means. A tag can be, for example, a polypeptide (e.g., an antibody or a fragment thereof), a nucleic acid molecule (e.g., a deoxyribonucleic acid (DNA), ribonucleic acid (RNA) molecule)) exhibiting at least a partial complementarity to a target nucleic acid sequence, or a small molecule configured to bind to a target epitope (e.g., a polypeptide sequence, a polynucleotide sequence, one or more polysaccharide moieties). In some cases, the tag can be functionalized (e.g., covalently or non-covalently) with one or more optically detectable moieties, such as, a dye (e.g., tetramethylrhodamine isothiocyanate (TRITC), Quantum Dots, CY3 and CY5), biotin-streptavidin conjugates, magnetic beads, fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. In some cases, the tag as disclosed herein, whether with or without the detectable moiety(ies), can be detected by, e.g., using photographic film or scintillation counters (e.g., for radiolabels), using photodetectors (e.g., for fluorescent markers), providing enzymes (e.g., for enzymatically modifiable substrates), etc. Alternatively, or in addition to, a tag can be a representation of any data comprising genetic information of a cell of interest, e.g., genetic information obtained after capturing one or more images of the cell.

The term “cluster” as used herein generally refers to a group of datapoints, such that datapoints in one group (e.g., a first cluster) are more similar to each other than datapoints of another group (e.g., a second cluster). A cluster can be a group of like datapoints (e.g., each datapoint representing a cell or an image of a cell) that are grouped together based on the proximity of the datapoints, to a measure of central tendency of the cluster. For example, a population of cells can be analyzed based on one or more morphological properties of each cell (e.g., by analyzing one or more images of each cell), and each cell can be plotted as a datapoint on a map base on the one or more morphological properties of each cell. Following, one or more clusters comprising a plurality of datapoints based on the proximity of the datapoints. The central tendency of each cluster can be measured by one or more algorithms (e.g., hierarchical clustering models, K-means algorithm, statistical distribution models, etc.). For instance, the measure of central tendency may be the arithmetic mean of the cluster, in which case the datapoints are joined together based on their proximity to the average value in the cluster (e.g., K-means clustering), their correlation, or their commonality.

The term “classifier” as used herein generally refers to an analysis model (e.g., a metamodel) that can be trained by using a learning model and applying learning algorithms (e.g., machine learning algorithms) on a training dataset (e.g., a dataset comprising examples of specific classes). In some cases, given a set of training examples/cases, each marked for belonging to a specific class (e.g., specific cell type or class), a training algorithm can build a classifier model capable of assigning new examples/cases (e.g., new datapoints of a cell or a group of cells) into one category or the other, e.g., to make the model a non-probabilistic classifier. In some cases, the classifier model can be capable of creating a new category to assign new examples/cases into the new category. In some cases, a classifier model can be the actual trained classifier that is generated based on the training model.

The term “cell type” as used herein generally refers to a kind, identity, or classification of cells according to one or more criteria, such as a tissue and species of origin, a differentiation state, whether or not they are healthy/normal or diseased, cell cycle stage, viability, etc. In non-limiting examples, the term “cell type” can refer specifically to any specific kind of cell, such as an embryonic stem cell, a neural precursor cell, a myoblast, a mesodermal cell, etc.

The term “cell state” as used herein generally refers to a specific state of the cell, such as but not limited to an activated cell, such as activated neuron or immune cell, resting cell, such as a resting neuron or immune cell, a dividing cell, quiescent cell, or a cell during any stages of the cell cycle.

The term “cell cycle” as used herein generally refers to the physiological and/or morphological progression of changes that cells undergo when dividing (e.g., proliferating). Examples of different phases of the cell cycle can include “interphase,” “prophase,” “metaphase,” “anaphase,” and “telophase”. Additionally, parts of the cell cycle can be “M (mitosis),” “S (synthesis),” “G0,” “G1 (gap 1)” and “G2 (gap 2)”. Furthermore, the cell cycle can include periods of progression that are intermediate to the above named phases.

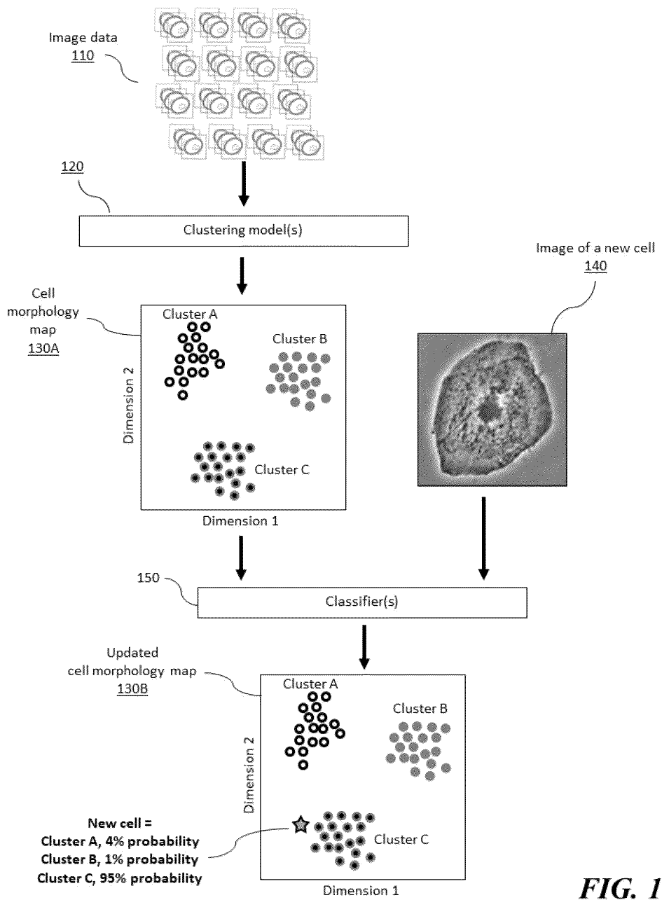

FIG. 1 schematically illustrates an example method for classifying a cell. The method can comprise processing image data 110 comprising tag-free images/videos of single cells (e.g., image data 110 consisting of tag-free images/videos of single cells). Various clustering analysis models 120 as disclosed herein can be used to process the image data 110 to extract one or more morphological properties of the cells from the image data 110, and generate a cell morphology map 130A based on the extracted one or more morphological properties. For example, the cell morphology map 130A can be generated based on two morphological properties as dimension 1 and dimension 2. The cell morphology map 130A can comprise one or more clusters (e.g., clusters A, B, and C) of datapoints, each datapoint representing an individual cell from the image data 110. The cell morphology map 130A and the clusters A-C therein can be used to train classifier(s) 150. Subsequently, a new image 140 of a new cell can be obtained and processed by the trained classifier(s) 150 to automatically extract and analyze one or more morphological features from the cellular image 140 and plot it as a datapoint on the cell morphology map 130A. Based on its proximity, correlation, or commonality with one or more of the morphologically-distinct clusters A-C on the cell morphology map 130A, the classifier(s) 150 can automatically classify the new cell. The classifier(s) 150 can determine a probability that the cell in the new image data 140 belongs to cluster C (e.g., the likelihood for the cell in the new image data 140 to share one or more commonalities and/or characteristics with cluster C more than with other clusters A/B). For example, the classifier(s) 150 can determine and report that the cell in the new image data 140 has a 95% probability of belonging to cluster C, 1% probability of belonging to cluster B, and 4% probability of belong to cluster A, solely based on analysis of the tag-free image 140 and one or more morphological features of the cell extracted therefrom.

An image and/or video (e.g., a plurality of images and/or videos) of one or more cells as disclosed herein (e.g., that of image data 110 in FIG. 1) can be captured while the cell(s) is suspended in a fluid (e.g., an aqueous liquid, such as a buffer) and/or while the cell(s) is moving (e.g., transported across a microfluidic channel). For example, the cell may not and need not be suspended is a gel-like or solid-like medium. The fluid can comprise a liquid that is heterologous to the cell(s)'s natural environment. For example, cells from a subject's blood can be suspended in a fluid that comprises (i) at least a portion of the blood and (ii) a buffer that is heterologous to the blood. The cell(s) may not be immobilized (e.g., embedded in a solid tissue or affixed to a microscope slide, such as a glass slide, for histology) or adhered to a substrate. The cell(s) may be isolated from its natural environment or niche (e.g., a part of the tissue the cell(s) would be in if not retrieved from a subject by human intervention) when the image and/or video of the cell(s) is captured. For example, the image and/or video may not and need not be from a histological imaging. The cell(s) may not and need not be sliced or sectioned prior to obtaining the image and/or video of the cell, and, as such, the cell(s) may remain substantially intact as a whole during capturing of the image and/or video.

When the image data is processed, e.g., to extract one or more morphological features of a cell, each cell image may be annotated with the extracted one or more morphological features and/or with information that the cell image belongs to a particular cluster (e.g., a probability).

The cell morphology map can be a visual (e.g., graphical) representation of one or more clusters of datapoints. The cell morphology map can be a 1-dimensional (1D) representation (e.g., based on one morphological property as one parameter or dimension) or a multi-dimensional representation, such as a 2-dimensional (2D) representation (e.g., based on two morphological properties as two parameters or dimensions), a 3-dimensional (3D) representation (e.g., based on three morphological properties as three parameters or dimensions), a 4-dimensional (4D) representation, etc. In some cases, one morphological properties of a plurality of morphological properties used for blotting the cell morphology map can be represented as a non-axial parameter (e.g., non-x, y, or z axis), such as, distinguishable colors (e.g., heatmap), numbers, letters (e.g., texts of one or more languages), and/or symbols (e.g., a square, oval, triangle, square, etc.). For example, a heatmap can be used as colorimetric scale to represent the classifier prediction percentages for each cell against a cell class, cell type, or cell state.

The cell morphology map can be generated based on one or more morphological features (e.g., characteristics, profiles, fingerprints, etc.) from the processed image data. Non-limiting examples of one or more morphological properties of a cell, as disclosed herein, that can be extracted from one or more images of the cell can include, but are not limited to (i) shape, curvature, size (e.g., diameter, length, width, circumference), area, volume, texture, thickness, roundness, etc. of the cell or one or more components of the cell (e.g., cell membrane, nucleus, mitochondria, etc.), (ii) number or positioning of one or more contents (e.g., nucleus, mitochondria, etc.) of the cell within the cell (e.g., center, off-centered, etc.), and (iii) optical characteristics of a region of the image(s) (e.g., unique groups of pixels within the image(s)) that correspond to the cell or a portion thereof (e.g., light emission, transmission, reflectance, absorbance, fluorescence, luminescence, etc.).

Non-limiting examples of clustering as disclosed herein can be hard clustering (e.g., determining whether a cell belongs to a cluster or not), soft clustering (e.g., determining a likelihood that a cell belongs to each cluster to a certain degree), strict partitioning clustering (e.g., determining whether each cell belongs to exactly one cluster), strict partitioning clustering with outliers (e.g., determining whether a cell can also belong to no cluster), overlapping clustering (e.g., determining whether a cell can belong to more than one cluster), hierarchical clustering (e.g., determining whether cells that belong to a child cluster can also belong to a parent cluster), and subspace clustering (e.g., determining whether clusters are not expected to overlap).

Cell clustering and/or generation of the cell morphology map, as disclosed herein, can be based on a single morphological property of the cells. Alternatively, cell clustering and/or generation the cell morphology map can be based on a plurality of different morphological properties of the cells. In some cases, the plurality of different morphological properties of the cells can have the same weight or different weights. A weight can be a value indicative of the importance or influence of each morphological property relative to one another in training the classifier or using the classifier to (i) generate one or more cell clusters, (ii) generate the cell morphology map, or (iii) analyze a new cellular image to classify the cellular image as disclosed herein. For example, cell clustering can be performed by having 50% weight on cell shape, 40% weight on cell area, and 10% weight on texture (e.g., roughness) of the cell membrane. In some cases, the classifier as disclosed herein can be configured to adjust the weights of the plurality of different morphological properties of the cells during analysis of new cellular image data, thereby to yield a most optimal cell clustering and cell morphology map. The plurality of different morphological properties with different weights can be utilized during the same analysis step for cell clustering and/or generation of the cell morphology map.

The plurality of different morphological properties can be analyzed hierarchically. In some cases, a first morphological property can be used as a parameter to analyze image data of a plurality of cells to generate an initial set of clusters. Subsequently, a second and different morphological property can be used as a second parameter to (i) modify the initial set of clusters (e.g., optimize arrangement among the initial set of clusters, re-group some clusters of the initial set of clusters, etc.) and/or (ii) generate a plurality of sub-clusters within a cluster of the initial set of clusters. In some cases, a first morphological property can be used as a parameter to analyze image data of a plurality of cells to generate an initial set of clusters, to generate a 1D cell morphology map. Subsequently, a second morphological property can be used as a parameter to further analyze the clusters of the 1D cell morphology map, to modify the clusters and generate a 2D cell morphology map (e.g., a first axis parameter based on the first morphological property and a second axis parameter based on the second morphological property).

In some cases of the hierarchical clustering as disclosed herein, an initial set of clusters can be generated based on an initial morphological feature that is extracted from the image data, and one or more clusters of the initial set of clusters can comprise a plurality of sub-clusters based on second morphological features or sub-features of the initial morphological feature. For example, the initial morphological feature can be stem cells (or not), and the sub-features can be different types of stem cells (e.g., embryonic stem cells, induced pluripotent stem cells, mesenchymal stem cells, muscle stem cells, etc.). In another example, the initial can be cancer cells (or not), and the sub-feature can be different types of cancer cells (e.g., sarcoma cells, sarcoma cells, leukemia cells, lymphoma cells, multiple myeloma cells, melanoma cells, etc.). In a different example, the initial can be cancer cells (or not), and the sub-feature can be different stages of the cancer cell (e.g., quiescent, proliferative, apoptotic, etc.).

Each datapoint can represent an individual cell or a collection of a plurality of cells (e.g., at least or up to about 2, 3, 4, 5, 6, 7, 8, 9, or 10 cells). Each datapoint can represent an individual image (e.g., of a single cell or a plurality of cells) or a collection of a plurality of images (e.g., at least or up to about 2, 3, 4, 5, 6, 7, 8, 9, or 10 images of the same single cell or different cells).

The cell morphology map can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500 clusters.

Each cluster as disclosed herein can comprise a plurality of sub-clusters, e.g., at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500 sub-clusters,

A cluster (or sub-cluster) can comprise datapoints representing cells of the same type/state. Alternatively, a cluster (or sub-cluster) can comprise datapoints representing cells of different types/states.

A cluster (or sub-cluster) can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500, at least or up to about 1,000, at least or up to about 2,000, at least or up to about 3,000, at least or up to about 4,000, at least or up to about 5,000, at least or up to about 10000, at least or up to about 50,000, or at least or up to about 100,000 datapoints.

Two or more clusters may overlap in a cell morphology map. Alternatively, no clusters may not overlap in a cell morphology map. In some cases, an allowable degree of overlapping between two or more clusters may be adjustable (e.g., manually or automatically by a machine learning algorithm) depending on the quality, condition, or size of data in the image data being processed.

A cluster (or sub-cluster) as disclosed herein can be represented with a boundary (e.g., a solid line or a dashed line). Alternatively, a cluster or sub-cluster may not and need not be represented with a boundary, and may be distinguishable from other cluster(s) sub-cluster(s) based on their proximity to one another.

A cluster (or sub-cluster) or a data comprising information about the cluster can be annotated based on one or more annotation schema (e.g., predefined annotation schema). Such annotation can be manual (e.g., by a user of the method or system disclosed herein) or automatically (e.g., by any of the machine learning algorithms disclosed herein). The annotation of the clustering can be related the one or more morphological properties of the cells that have been analyzed (e.g., cell shape, cell area, optical characteristic(s), etc.) to generate the cluster or assign one or more datapoints to the cluster. Alternatively, the annotation of the clustering can be related to information that has not been used or analyzed to generate the cluster or assign one or more datapoints to the cluster (e.g., genomics, transcriptomics, or proteomics, etc.). In such case, the annotation can be utilized to add additional “layers” of information to each cluster.

In some cases, an interactive annotation tool can be provided that permits one or more users to modify any process of the method described herein. For example, the interactive annotation tool can allow a user to curate, verify, edit, and/or annotate the morphologically-distinct clusters. In another example, the interactive annotation tool can process the image data, extract one or more morphological features from the image data, and allow the user to select one or more of the extracted morphological features to be used as a basis to generate the clusters and/or the cell morphology map. After the generation of the clusters and/or the cell morphology map, the interactive annotation tool can allow the user to annotate each cluster and/or the cell morphology map using (i) a predefined annotation schema or (ii) a new, user-defined annotation schema. In another example, the interactive annotation tool can allow user to assign different weights to different morphological features for the clustering and/or map plotting. In another example, the interactive annotation tool can allow user to select with imaging data (or which cells) to be used and/or which imaging data (or which cells, cell clumps, artifacts, or debris) to be discarded, for the clustering and/or map plotting. A user can manually identify incorrectly clustered cells, or the machine learning algorithm can provide probability or correlation value of cells within each cluster and identify any outlier (e.g., a datapoint that would change the outcome of the probability/correlation value of the cluster(s) by a certain percentage value). Thus, the user can choose to move the outliers via the interactive annotation tool to further tune the cell morphology map, e.g., to yield a “higher resolution” map.

One or more cell morphology maps as disclosed herein can be used to train one or more classifiers (e.g., at least or up to about 1, 2, 3, 4, 5, 6, 7, 8, 9, 10, or more classifiers) as disclosed herein. Each classifier can be trained to analyze one or more images of a cell (e.g., to extract one or more morphological features of the cell) and categorize (or classify) the cell into one or more determined class or categories of a cell (e.g., based on a type of state of the cell). Alternatively, the classifier can be trained to create a new category to categorize (or classify) the cell into the new category, e.g., when determining that the cell is morphologically distinct than any pre-existing categories of other cells.

The machine learning algorithm as disclosed herein can be configured to extract one or more morphological feature of a cell from the image data of the cell. The machine learning algorithm can form a new data set based on the extracted morphological features, and the new data set may not and need not contain the original image data of the cell. In some examples, replicas of the original images in the image data can be stored in a database disclosed herein, e.g., prior to using any of the new images for training, e.g., to keep the integrity of the images of the image data. In some examples, processed images of the original images in the image data can be stored in a database disclosed herein during or subsequent to the classifier training. In some cases, any of the newly extracted morphological features as disclosed herein can be utilized as new molecular markers for a cell or population of cells of interest to the user. As cell analysis platform as disclosed herein can be operatively coupled to one or more databases comprising non-morphological data of cells processed (e.g., genomics data, transcriptomics data, proteomics data, metabolomics data), a selected population of cells exhibiting the newly extracted morphological feature(s) can be further analyzed by their non-morphological properties to identify proteins or genes of interest that are common in the selected population of cells but not in other cells, thereby determining such proteins or genes of interest to be new molecular markers that can be used to identify such selected population of cells.

In some cases, a classifier can be trained by applying machine learning algorithms on at least a portion of one or more cell morphology maps as disclosed herein as a training dataset. Non-limiting examples of machine learning algorithms for training a classifier can include supervised learning, unsupervised learning, semi-supervised learning, reinforcement learning, self-learning, feature learning, anomaly detection, association rules, etc. In some cases, a classifier can be trained by using one or more learning models on such training dataset. Non-limiting examples of learning models can include artificial neural networks (e.g., convolutional neural networks, U-net architecture neural network, etc.), backpropagation, boosting, decision trees, support vector machines, regression analysis, Bayesian networks, genetic algorithms, kernel estimators, conditional random field, random forest, ensembles of classifiers, minimum complexity machines (MCM), probably approximately correct learning (PACT), etc.

In some cases, the neural networks are designed by the modification of neural networks such as AlexNet, VGGNet, GoogLeNet, ResNet (residual networks), DenseNet, and Inception networks. In some examples, the enhanced neural networks are designed by modification of ResNet (e.g. ResNet 18, ResNet 34, ResNet 50, ResNet 101, and ResNet 152) or inception networks. In some aspects, the modification comprises a series of network surgery operations that are mainly carried out to improve including inference time and/or inference accuracy.

The machine learning algorithm as disclosed herein can utilize one or more clustering algorithms to determine that objects in the same cluster can be more similar (in one or more morphological features) to each other than those in other clusters. Non-limiting examples of the clustering algorithms can include, but are not limited to, connectivity models (e.g., hierarchical clustering), centroid models (e.g. K-means algorithm), distribution models (e.g., expectation-maximization algorithm), density models (e.g., density-based spatial clustering of applications with noise (DB SCAN), ordering points to identify the clustering structure (OPTICS)), subspace models (e.g., biclustering), group models, graph-based models (e.g., highly connected subgraphs (HCS) clustering algorithms), single graph models, and neural models (e.g., using unsupervised neural network). The machine learning algorithm can utilize a plurality of models, e.g., in equal weights or in different weights.

In some cases, unsupervised and self-supervised approaches can be used to expedite labeling of image data of cells. For the case of unsupervised, an embedding for a cell image can be generated. For example, the embedding can be a representation of the image in a space with reduced dimensions than the original image data. Such embeddings can be used to cluster images that are similar to one another. Thus, the labeler can be configured to batch-label the cells and increase the throughput as compared to manually labeling one or more cells.

In some cases, for the case of self-supervised learning, additional meta information (e.g., additional non-morphological information) about the sample (e.g., what disease is known or associated with the patient who provided the sample) can be used for labeling of image data of cells.

In some cases, embedding generation can use a neural net trained on predefined cell types. To generate the embeddings described herein, an intermediate layer of the neural net that is trained on predetermined image data (e.g., image data of known cell types and/or states) can be used. By providing enough diversity in image data/sample data to the trained model/classifier, this method can provide an accurate way to cluster future cells.

In some cases, embedding generation can use neural nets trained for different tasks. To generate the embeddings described herein, an intermediate layer of the neural net that is trained for a different task (e.g., a neural net that is trained on a canonical dataset such as ImageNet). Without wishing to be bound by theory, this can allow to focus on features that matter for image classification (e.g., edges and curves) while removing a bias that may otherwise be introduced in labeling the image data.

In some cases, autoencoders can be used for embedding generation. To generate the embeddings described herein, autoencoders can be used, in which the input and the output can be substantially the same image and the squeeze layer can be used to extract the embeddings. The squeeze layer can force the model to learn a smaller representation of the image, which smaller representation may have sufficient information to recreate the image (e.g., as the output).

In some cases, for clustering-based labeling of image data or cells, as disclosed herein, an expanding training data set can be used. With the expanding training data set, one or more revisions of labeling (e.g., manual relabeling) may be needed to, e.g., avoid the degradation of model performance due to the accumulated effect of mislabeled images. Such manual relabeling may be intractable on a large scale and ineffective when done on a random subset of the data. Thus, to systematically surface images for potential relabeling, for example, similar embedding-based clustering can be used to identify labeled images that may cluster with members of other classes. Such examples are likely to be enriched for incorrect or ambiguous labels, which can be removed (e.g., automatically or manually).

In some cases, adaptive image augmentation can be used. In order to make the models and classifiers disclosed herein more robust to artifacts in the image data, (1) one or more images with artifacts can be identified, and (2) such images identified with artifacts can be added to training pipeline (e.g., for training the model/classifier). Identifying the image(s) with artifacts can comprise: (1a) while imaging cells, one or more additional sections of the image frame can be cropped, which frame(s) being expected to contain just the background without any cell; (2a) the background image can be checked for any change in one or more characteristics (e.g., optical characteristics, such as brightness); and (3a) flagging/labeling one or more images that have such change in the characteristic(s). Adding the identified images to training pipeline can comprise: (2a) adding the one or more images that have been flagged/labeled as augmentation by first calculating an average feature of the changed characteristic(s) (e.g., the background median color); (2b) creating a delta image by subtracting the average feature from the image data (e.g., subtracting the median for each pixel of the image); and (3c) adding the delta image to the training pipeline.

One or more dimension of the cell morphology map can be represented by various approaches (e.g., dimensionality reduction approaches), such as, for example, principal component analysis (PCA), multidimensional scaling (MDS), t-distributed stochastic neighbor embedding (t-SNE), and uniform manifold approximation and projection (UMAP). For example, UMAP can be a machine learning technique for dimension reduction. UMAP can be constructed from a theoretical framework based in Riemannian geometry and algebraic topology. UMAP can be utilized for a practical scalable algorithm that applies to real world data, such as morphological properties of one or more cells.

The cell morphology map as disclosed herein can comprise an ontology of the one or more morphological features. The ontology can be an alternative medium to represent a relationship among various datapoints (e.g., each representing a cell) analyzed from an image data. For example, an ontology can be a data structure of information, in which nodes can be linked by edges. An edge can be used to define a relationship between two nodes. For example, a cell morphology map can comprise a cluster comprising sub-clusters, and the relationship between the cluster and the sub-clusters can be represented in an nodes/edges ontology (e.g., an edge can be used to describe the relationship as a subclass of, genus of, part of, stem cell of, differentiated from, progeny of, diseased state of, targets, recruits, interacts with, same tissue, different tissue, etc.).

In some cases, one-to-one morphology to genomics mapping can be utilized. An image of a single cell or images of multiple “similar looking” cells can be mapped to its/their molecular profile(s) (e.g., genomics, proteomics, transcriptomics, etc.). In some examples, classifier-based barcoding can be performed. Each sorting event (e.g., positive classifier) can push the sorted cell(s) into an individual well or droplet with a unique barcode (e.g., nucleic acid or small molecule barcode). The exact barcode(s) used for that individual classifier positive event can be recorded and tracked. Following, the cells can be lysed and molecularly analyzed together with the barcode(s). The result of the molecular analysis can then be mapped (e.g., one-to-one) to the image(s) of the individual (or ensemble of) sorted cell(s) captured while the cell(s) was/were flowing in the flow channel. In some examples, class-based sorting can be utilized. Cells that are classified in the same class based at least on their morphological features can be sorted into a single well or droplet with a pre-determined barcoded material, and the cells can be lysed, molecularly analyzed, then any molecular information can be used for the one-to-one mapping as disclosed herein.

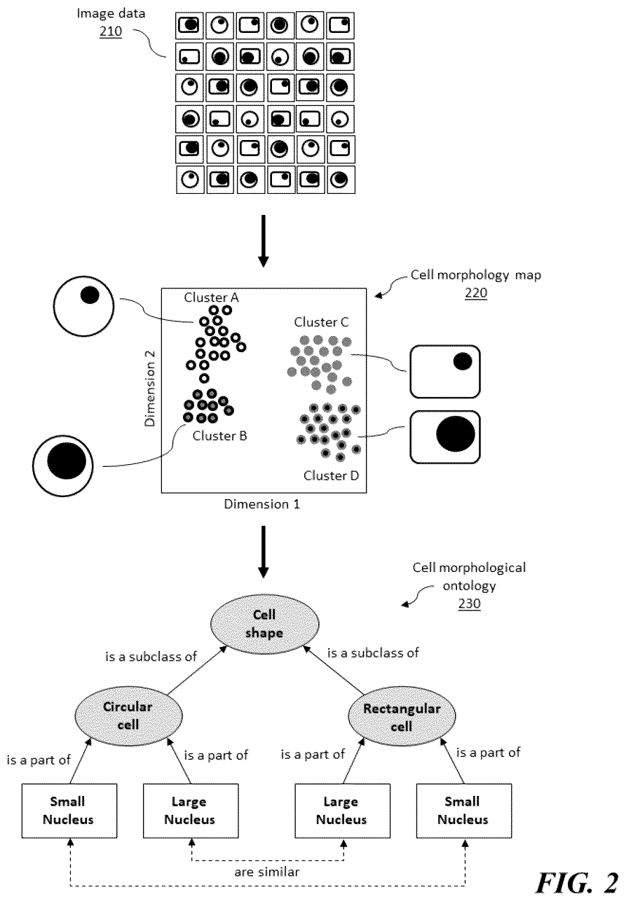

FIG. 2 schematically illustrates different ways of representing analysis data of image data of cells. Tag-free image data 210 of cells (e.g., circular cells and square cells) having different nuclei (e.g., small nucleus and large nucleus) can be analyzed by any of the methods disclosed herein (e.g., based on extraction of one or more morphological features). For example, any of the classifier(s) disclosed herein can be used to analyze and plot the image data 210 into a cell morphology map 220, comprising four distinguishable clusters: cluster A (circular cell, small nucleus), cluster B (circular cell, large nucleus), cluster C (square cell, small nucleus), and cluster D (square cell, large nucleus). The classifier(s) can also represent the analysis in a cell morphological ontology 230, in which a top node (“cell shape”) can be connected to two sub-nodes (“circular cell” and rectangular cell”) via an edge (“is a subclass of”) to define the relationship between the nodes. Each sub-node can also connected to its own sub-nodes (“small nucleus” and “large nucleus”) via an edge (“is a part of”) to define their relationships. The sub-nodes (e.g., “small nucleus” and “large nucleus”) can also be connected via one or more edges (“are similar”) to further define their relationship.

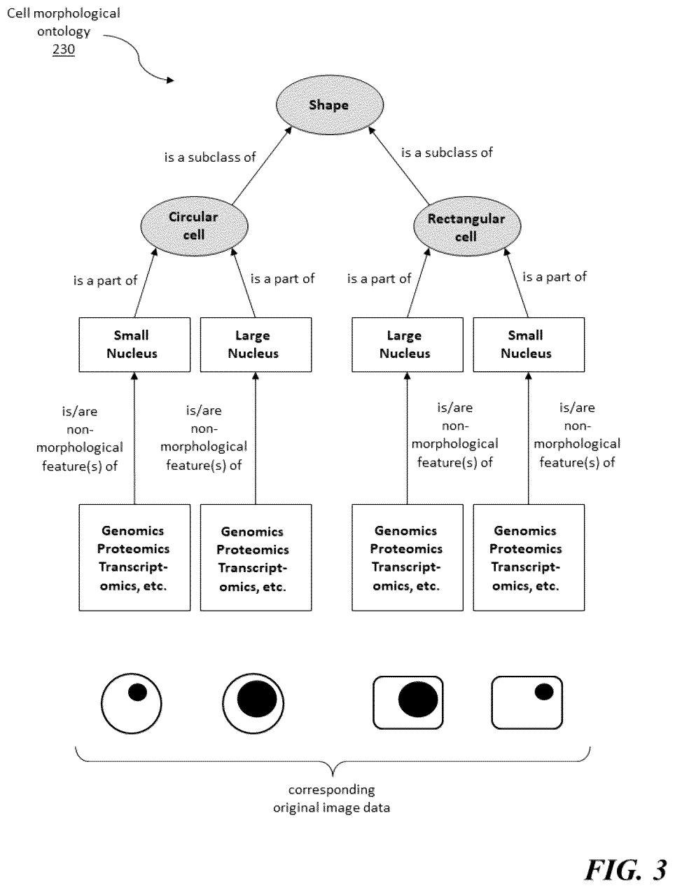

The cell morphology map or cell morphological ontology as disclosed herein can be further annotated with one or more non-morphological data of each cell. As shown in FIG. 3, the ontology 230 from FIG. 2 can be further annotated with information about the cells that may not be extractable from the image data used to classify the cells (e.g., molecular profiles obtained via molecular barcodes, as disclosed herein). Non-limiting examples of such non-morphological data can be from additional treatment and/or analysis, including, but not limited to, cell culture (e.g., proliferation, differentiation, etc.), cell permeabilization and fixation, cell staining by a probe, mass cytometry, multiplexed ion beam imaging (MIBI), confocal imaging, nucleic acid (e.g., DNA, RNA) or protein extraction, polymerase chain reaction (PCR), target nucleic acid enrichment, sequencing, sequence mapping, etc.

Examples of the probe used for cell staining (or tagging) may include, but are not limited to, a fluorescent probe (e.g., for staining chromosomes such as X, Y, 13, 18 and 21 in fetal cells), a chromogenic probe, a direct immunoagent (e.g. labeled primary antibody), an indirect immunoagent (e.g., unlabeled primary antibody coupled to a secondary enzyme), a quantum dot, a fluorescent nucleic acid stain (such as DAPI, Ethidium bromide, Sybr green, Sybr gold, Sybr blue, Ribogreen, Picogreen, YoPro-1, YoPro-2 YoPro-3, YOYo, Oligreen acridine orange, thiazole orange, propidium iodine, or Hoeste), another probe that emits a photon, or a radioactive probe.

In some cases, the instrument(s) for the additional analysis may comprise a computer executable logic that performs karyotyping, in situ hybridization (ISH) (e.g., florescence in situ hybridization (FISH), chromogenic in situ hybridization (CISH), nanogold in situ hybridization (NISH)), restriction fragment length polymorphism (RFLP) analysis, polymerase chain reaction (PCR) techniques, flow cytometry, electron microscopy, quantum dot analysis, or detects single nucleotide polymorphisms (SNPs) or levels of RNA.

Analysis of the image data (e.g., extracting one or more morphological features form the image data, determining clustering and/or cell morphology map based on the image data, etc.) can be performed (e.g., automatically) within less than about 1 hour, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 50 seconds, 40 seconds, 30 seconds, 20 seconds, 10 seconds, 5 seconds, 1 second, or less. In some cases, such analysis can be performed in real-time.

One or more morphological features utilized for generating the clusters or the cell morphology map, as disclosed herein, can be selected automatically (e.g., by one or more machine learning algorithms) or, alternatively, selected manually by a user via a user interface (e.g., graphical user interface (GUI)). The GUI can show visualization of, for example, (i) the one or more morphological parameters extracted from the image data (e.g., represented as images, words, symbols, predefined codes, etc.), (ii) the cell morphology map comprising one or more clusters, or (iii) the cell morphological ontology. The user can select, via the GUI, which morphological parameter(s) to be used to generate the clusters and the cell morphological map prior to actual generation of the clusters and the cell morphological map. The user can, upon seeing or receiving a report about the generated clusters and the cell morphological map, retroactively modify the types of morphological parameter(s) to use, thereby to (i) modify the clustering or the cell morphological mapping and/or (ii) create new cluster(s) or new cell morphological map(s). In some cases, the user can select one or more regions to be excluded or included for further analysis or further processing of the cells (e.g., sorting in the future or in real-time). For example, a microfluidic system as disclosed herein can be utilized to capture image(s) of each cell from a population of cells, and any of the methods disclosed herein can be utilized to analyze such image data to generate a cell morphology map comprising clusters representing the population of cells. The user can select one or more clusters or sub-clusters to be sorted, and the input can be provided to the microfluidic system to sort at least a portion of the cells into one or more sub-channels of the microfluidic system (e.g., in real-time) accordingly. Alternatively, the user can select one or more clusters or sub-clusters to be excluded during sorting (e.g., to get rid of artifacts, debris, or dead cells), and the input can be provided to the microfluidic system to sort at least a portion of the cells into one or more sub-channels of the microfluidic system (e.g., in real-time) accordingly without such artifacts, debris, or dead cells.

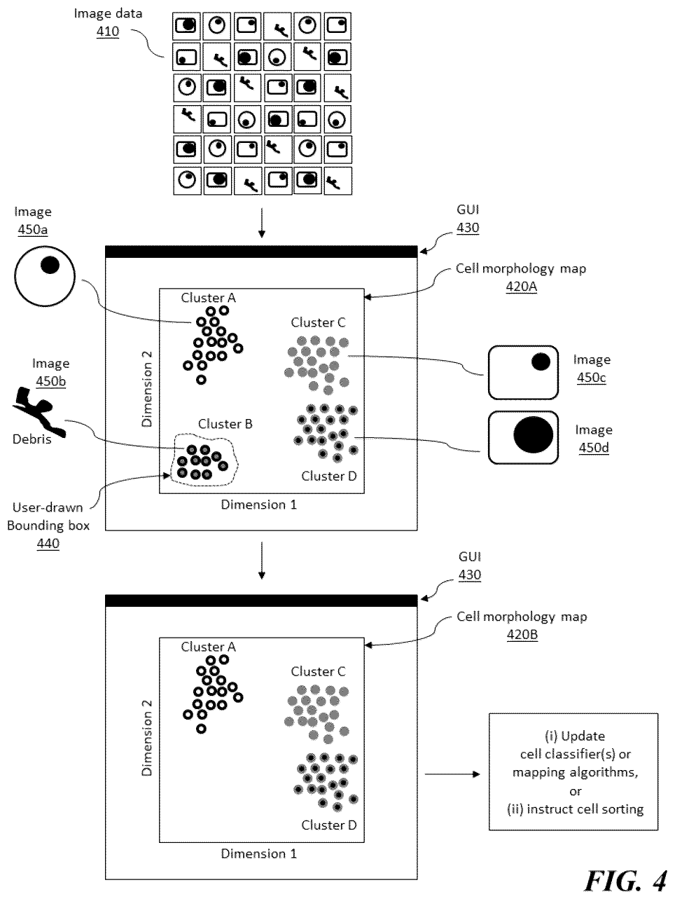

FIG. 4 schematically illustrates a method for a user to interact (e.g., via GUI) with any one of the methods disclosed herein. Image data 410 of a plurality of cells can be processed, via any one of the methods disclosed herein, to generate a cell morphology map 420A that represents the plurality of cells as datapoints in different clusters A, B, C, and D. The cell morphology map 420A can be displayed to the user via the GUI 430. The user can select each cluster or a datapoint within each cluster to visualize one or more images 450a, b, c, or d of the cells classified into the cluster. Upon visualization of the images, the user can draw a box 440 (e.g., via any user-defined shape and/or size) around one or more datapoints or around a cluster. For example, the user can draw a box 440 around a cluster of “debris” datapoints, to, e.g., remove the selected cluster and generate a new cell morphology map 420B. The user input can be used to update cell classifying algorithms (e.g., one or more classifier(s) as disclosed herein), mapping algorithms, cell flowing mechanism (e.g., velocity of cells, positioning of the cells within a flow channel, adjusting imaging focal length/plane of one or more sensors/cameras of an imaging module (also referred to as an imaging device herein) that captures one or more images/videos of cells flowing through the flow cell, etc.), cell sorting mechanisms in the flow channel, cell sorting instructions in the flow channel, etc. For example, upon the user's selection, the classifier can be trained to identify one or more common morphological features within the selected datapoints (e.g., features that distinguish the selected datapoints from the unselected data). Features of the selected group can be used to further identify other cells from other samples having similar feature(s) for further analysis or discard cells having similar feature(s), e.g., for cell sorting.

The present disclosure also describes a cell analysis platform, e.g., for analyzing or classifying a cell. The cell analysis platform can be a product of any one of the methods disclosed herein. Alternatively, or in addition to, the cell analysis platform can be used as a basis to execute any one of the methods disclosed herein. For example, the cell analysis platform can be used to process image data comprising tag-free images of single cells to generate a new cell morphology map of various cell clusters. In another example, the cell analysis platform can be used to process image data comprising tag-free images of single cells to compare the cell to pre-determined (e.g., pre-analyzed) images of known cells or cell morphology map(s), such that the single cells from the image data can be classified, e.g., for cell sorting.

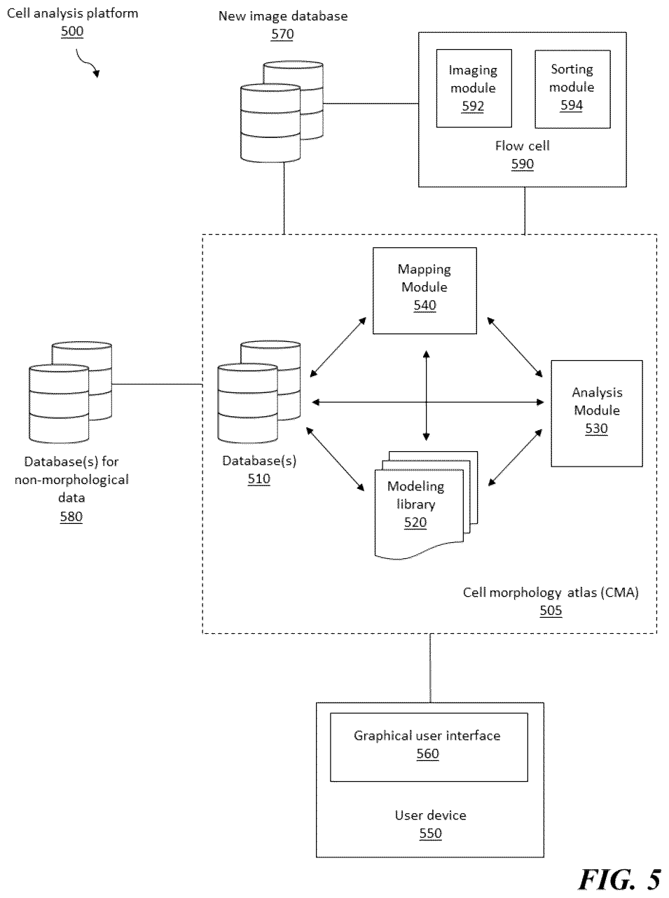

FIG. 5 illustrates an example cell analysis platform (e.g., machine learning/artificial intelligence platform) for analyzing image data of one or more cells. The cell analysis platform 500 can comprise a cell morphology atlas (CMA) 505. The CMA 505 can comprise a database 510 having a plurality of annotated single cell images that are grouped into morphologically-distinct clusters (e.g., represented a texts, as cell morphology map(s), or cell morphological ontology(ies)) corresponding to a plurality of classifications (e.g., predefined cell classes). The CMA 505 can comprise a modeling unit comprising one or more models (e.g., modeling library 520 comprising, such as, one or more machine learning algorithms disclosed herein) that are trained and validated using datasets from the CMA 505, to process image data comprising images/videos of one or more cells to identify different cell types and/or states based at least on morphological features. The CMA 505 can comprise an analysis module 530 comprising one or more classifiers as disclosed herein. The classifier(s) can uses one or more of the models from the modeling library 520 to, e.g., (1) classify one or more images taken from a sample, (2) assess a quality or state of the sample based on the one or more images, (3) map one or more datapoints representing such one or more images onto a cell morphology map (or cell morphological ontology) via using a mapping module 540. The CMA 505 can be operatively coupled to one or more additional database 570 to receive the image data comprising the images/videos of one or more cells. For example, the image data from the database 570 can be obtained from an imaging module 592 of a flow cell 590, which can also be operatively coupled to the CMA 505. The flow cell can direct flow of a sample comprising or suspected of comprising a target cell, and capture one or more images of contents (e.g., cells) within the sample by the imaging module 592. Any image data obtained by the imaging module 592 can be transmitted directly to the CMA 505 and/or to the new image database 570. Alternatively or in addition to, the CMA 505 can be operatively coupled to one or more additional databases 580 comprising non-morphological data of any of the cells (e.g., genomics, transcriptomics, or proteomics, etc.), e.g., to further annotate any of the datapoint, cluster, map, ontology, images, as disclosed herein. The CMA 505 can be operatively coupled to a user device 550 (e.g., a computer or a mobile device comprising a display) comprising a GUI 560 for the user to receive information from and/or to provide input (e.g., instructions to modify or assist any portion of the method disclosed herein). Any classification made by the CMA and/or the user can be provided as an input to the sorting module 594 of the flow cell 590. Based on the classification, the sorting module can determine, for example, (i) when to activate one or more sorting mechanisms at the sorting junction of the flow cell 590 to sort one or more cells of interest, (ii) which sub-channel of a plurality of sub-channels to direct each single cell for sorting. In some cases, the sorted cells can be collected for further analysis, e.g., downstream molecular assessment and/or profiling, such as genomics, transcriptomics, proteomics, metabolomics, etc.

Any of the methods or platforms disclosed herein can be used as a tool that permits a user to train one or more models (e.g., from the modeling library) for cell clustering and/or cell classification. For example, a user may provide initial image dataset of a sample to the platform, and the platform may process the initial set of image data. Based on the processing, the platform can determine a number of labels and/or an amount of data that the user needs to train the one or more models, based on the initial image dataset of the sample. In some examples, the platform can determine that the initial set of image data can be insufficient to provide an accurate cell classification or cell morphology map. For example, the platform can plot an initial cell morphology map and recommend to the user the number of labels and/or the amount of data needed to for enhanced processing, classification, and/or sorting, based on proximity (or separability), correlation, or commonality of the datapoints in the map (e.g., whether there is no distinguishable clusters within the map, whether the clusters within the map are too close to each other, etc.). In another example, the platform can allow the user to select different model (e.g., clustering model) or classifier, different combinations of models or classifiers, to re-analyze the initial set of image data.

Any of the methods or platforms disclosed herein can be used to determine quality or state of the image(s) of the cell, that of the cell, or that of a sample comprising the cell. The quality or state of the cell can be determined at a single cell level. Alternatively, the quality or state of the cell can be determined at an aggregate level (e.g., as a whole sample, or as a portion of the sample). The quality or state can be determined and reported based on, e.g., a number system (e.g., a number scale from 1 to 10, a percentage scale from 1% to 100%), a symbolic system, or a color system. For example, the quality or state can be indicative of a preparation or priming condition of the sample (e.g., whether the sample has a sufficient number of cells, whether the sample has too much artifacts, debris, etc.) or indicative of a viability of the sample (e.g., whether the sample has an amount of “dead” cells above a predetermined threshold).

Any of the methods or platforms disclosed herein can be used to sort cells in silico (e.g., prior to actual sorting of the cells using a microfluidic channel). The in silico sorting can be, e.g., to discriminate among and/or between, e.g., multiple different cell types (e.g., different types of cancer cells, different types of immune cells, etc.), cell states, cell qualities. The methods and platforms disclosed herein can utilize pre-determined morphological properties (e.g., provided in the platform) for the discrimination. Alternatively or in addition to, newly abstracted morphological properties can be abstracted (e.g., generated) based on the input data for the discrimination. In some cases, new model(s) and/or classifier(s) can be trained or generated to process the image data. In some cases, the newly abstracted morphological properties can be used to discriminate among and/or between, e.g., multiple different cell types, cell states, cell qualities that are known. Alternatively or in addition to, the newly abstracted morphological properties can be used to create new class (or classifications) to sort the cells (e.g., in silico or via the microfluidic system). The newly abstracted morphological properties as disclosed herein may enhance accuracy or sensitivity of cell sorting (e.g., in silico or via the microfluidic system).

Subsequent to the in silico sorting of the cells, the actual cell sorting of the cells (e.g., via the microfluidic system or flow cell) based on the in silico sorting can be performed within less than about 1 hours, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 50 seconds, 40 seconds, 30 seconds, 20 seconds, 10 seconds, 5 seconds, 1 second, or less. In some cases, the in silico sorting and the actual sorting can occur in real-time.

In any of the methods or platforms disclosed herein, the model(s) and/or classifier(s) can be validated (e.g., for the ability to demonstrate accurate cell classification performance). Non-limiting examples of validation metrics that can be utilized can include, but are not limited to, threshold metrics (e.g., accuracy, F-measure, Kappa, Macro-Average Accuracy, Mean-Class-Weighted Accuracy, Optimized Precision, Adjusted Geometric Mean, Balanced Accuracy, etc.), the ranking methods and metrics (e.g., receiver operating characteristics (ROC) analysis or “ROC area under the curve (ROC AUC)”), and the probabilistic metrics (e.g., root-mean-squared error). For example, the model(s) or classifier(s) can be determined to be balanced or accurate when the ROC AUC is greater than 0.5, greater than 0.55, greater than 0.6, greater than 0.65, greater than 0.7, greater than 0.75, greater than 0.8, greater than 0.85, greater than 0.9, greater than 0.91, greater than 0.92, greater than 0.93, greater than 0.94, greater than 0.95, greater than 0.96, greater than 0.97, greater than 0.98, greater than 0.99, or more.

In any of the methods or platforms disclosed herein, the image(s) of the cell(s) can be obtained when the cell(s) are prepared and diluted in a sample (e.g., a buffer sample). The cell(s) can be diluted, e.g., in comparison to real-life concentrations of the cell in the tissue (e.g., solid tissue, blood, serum, spinal fluid, urine, etc.) to a dilution concentration. The methods or platforms disclosed herein can be compatible with a sample (e.g., a biological sample or derivative thereof) that is diluted by a factor of about 500 to about 1,000,000. The methods or platforms disclosed herein can be compatible with a sample that is diluted by a factor of at least about 500. The methods or platforms disclosed herein can be compatible with a sample that is diluted by a factor of at most about 1,000,000. The methods or platforms disclosed herein can be compatible with a sample that is diluted by a factor of about 500 to about 1,000, about 500 to about 2,000, about 500 to about 5,000, about 500 to about 10,000, about 500 to about 20,000, about 500 to about 50,000, about 500 to about 100,000, about 500 to about 200,000, about 500 to about 500,000, about 500 to about 1,000,000, about 1,000 to about 2,000, about 1,000 to about 5,000, about 1,000 to about 10,000, about 1,000 to about 20,000, about 1,000 to about 50,000, about 1,000 to about 100,000, about 1,000 to about 200,000, about 1,000 to about 500,000, about 1,000 to about 1,000,000, about 2,000 to about 5,000, about 2,000 to about 10,000, about 2,000 to about 20,000, about 2,000 to about 50,000, about 2,000 to about 100,000, about 2,000 to about 200,000, about 2,000 to about 500,000, about 2,000 to about 1,000,000, about 5,000 to about 10,000, about 5,000 to about 20,000, about 5,000 to about 50,000, about 5,000 to about 100,000, about 5,000 to about 200,000, about 5,000 to about 500,000, about 5,000 to about 1,000,000, about 10,000 to about 20,000, about 10,000 to about 50,000, about 10,000 to about 100,000, about 10,000 to about 200,000, about 10,000 to about 500,000, about 10,000 to about 1,000,000, about 20,000 to about 50,000, about 20,000 to about 100,000, about 20,000 to about 200,000, about 20,000 to about 500,000, about 20,000 to about 1,000,000, about 50,000 to about 100,000, about 50,000 to about 200,000, about 50,000 to about 500,000, about 50,000 to about 1,000,000, about 100,000 to about 200,000, about 100,000 to about 500,000, about 100,000 to about 1,000,000, about 200,000 to about 500,000, about 200,000 to about 1,000,000, or about 500,000 to about 1,000,000. The methods or platforms disclosed herein can be compatible with a sample that is diluted by a factor of about 500, about 1,000, about 2,000, about 5,000, about 10,000, about 20,000, about 50,000, about 100,000, about 200,000, about 500,000, or about 1,000,000.

In any of the methods or platforms disclosed herein, the classifier can generate a prediction probability (e.g., based on the morphological clustering and analysis) that an individual cell or a cluster of cells belongs to a cell class (e.g., within a predetermined cell class provided in the CMA as disclosed herein), e.g., via a reporting module. The reporting module can communicate with the user via a GUI as disclosed herein. Alternatively or in addition to, the classifier can generate a prediction vector that an individual cell or a cluster of cells belongs to a plurality of cell classes (e.g., a plurality of all of predetermined cell classes from the CMA as disclosed herein). The vector can be 1D (e.g., a single row of different cell classes), 2D (e.g., two dimensions, such as tissue origin vs. cell type), 3D, etc. In some cases, based on processing and analysis of image data obtained from a sample, the classifier can generate a report showing a composition of the sample, e.g., a distribution of one or more cell types, each cell type indicated with a relative proportion within the sample. Each cell of the sample can also be annotated with a most probable cell type and one or more less probably cell types.

Any one of the methods and platforms disclosed herein can be capable of processing image data of one or more cells to generate one or more morphometric maps of the one or more cells. Non-limiting examples of morphometric models can be utilized to analyze one or more images of single cells (or cell clusters) can include, e.g., simple morphometrics (e.g., based on lengths, widths, masses, angles, ratios, areas, etc.), landmark-based geometric morphometrics (e.g., spatial information, intersections, etc. of one or more components of a cell), procrustes-based geometric morphometrics (e.g., by removing non-shape information that is altered by translation, scaling, and/or rotation from the image data), Euclidean distance matrix analysis, diffeomorphometry, and outline analysis. The morphometric map(s) can be multi-dimensional (e.g., 2D, 3D, etc.). The morphometric map(s) can be reported to the user via the GUI.

Any of the methods or platforms disclosed herein (e.g., the analysis module) can be used to process, analyze, classify, and/or compare two or more samples (e.g., at least 2, 3, 4, 5, 6, 7, 8, 9, 10, or more test samples). The two or more samples can each be analyzed to determine a morphological profile (e.g., a cell morphology map) of each sample. For example, the morphological profiles of the two or more samples can be compared for identifying a disease state of a patient's sample in comparison to a health cohort's sample or a sample of image data representative of a disease of interest. In another example, the morphological profiles of the two or more samples can be compared to monitor a progress of a condition of a subject, e.g., comparing first image data of a first set of cells from a subject before a treatment (e.g., a test drug candidate, chemotherapy, surgical resection of solid tumors, etc.) and second image data of a second set of cells from the subject after the treatment. The second set of cells can be obtained from the subject at least about 1 week, at least about 2 weeks, at least about 3 weeks, at least about 4 weeks, at least about 2 months, or at least about 3 months subsequent to obtaining the first set of cells from the subject. In a different example, the morphological profiles of the two or more samples can be compared to monitor effects of two or more different treatment options (e.g., different test drugs) in two or more different cohorts (e.g., human subjects, animal subjects, or cells being tested in vitro/ex vivo). Accordingly, the systems and methods disclosed herein can be utilized (e.g., via sorting or enrichment of a cell type of interest or a cell exhibiting a characteristic of interest) to select a drug and/or a therapy that yields a desired effect (e.g., a therapeutic effect greater than equal to a threshold value).

Any of the platforms disclosed herein (e.g., cell analysis platform) can provide an inline end-to-end pipeline solution for continuous labeling and/or sorting of multiple different cell types and/or states based at least in part on (e.g., based solely on) morphological analysis of imaging data provided. A modeling library used by the platform can be scalable for large amount of data, extensible (e.g., one or more models or classifiers modified), and/or generalizable (e.g., more resistant to data perturbations—such as artifacts, debris, random objects in the background, image/video distortions—between samples. Any of the modeling library may be removed or updated with new model automatically by the machine learning algorithms or artificial intelligence, or by the user.

Any of the methods and platforms disclosed herein can adjust one or more parameters of the microfluidic system as disclosed herein. As cells are flowing through a flow channel, an imaging module (e.g., sensors, cameras) can capture image(s)/video(s) of the cells and generate new image data. The image data can be processed and analyzed (e.g., in real-time) by the methods and platforms of the present disclosure to train a model (e.g., machine learning model) to determine whether or not one or more parameters of the microfluidic system.

In some cases, the model(s) can determine that the cells are flowing too fast or too slow, and send an instruction to the microfluidic system to adjust (i) the velocity of the cells (e.g., via adjusting velocity of the fluid medium carrying the cells) and/or (ii) image recording rate of a camera that is capturing images/videos of cells flowing through the flow channel.

In some cases, the model(s) can determine that the cells are in-focus or out-of-focus in the images/videos, and send an instruction to the microfluidic system to (i) adjust a positioning of the cells within the flow cell (e.g., move the cell towards or away from the center of the flow channel via, for example, hydrodynamic focusing and/or inertial focusing) and/or (ii) adjust a focal length/plane of the camera that is capturing images/videos of cells flowing through the flow channel. Adjusting the focal length/plane can be performed for the same cell that has been analyzed (e.g., adjusting focal length/plane of a camera that is downstream) or a subsequent cell. Adjusting the focal length/plane can enhance clarity or reduce blurriness in the images. The focal length/plane can be adjusted based on a classified type or state of the cell. In some examples, adjusting the focal length/plane can allow enhanced focusing/clarity on all parts of the cell. In some examples, adjusting the focal length/plane can allow enhanced focusing/clarity on different portions (but not all parts) of the cell. Without wishing to be bound by theory, out-of-focus images may be usable for any of the methods disclosed herein to extract morphological feature(s) of the cell that otherwise may not be abstracted from in-focus images, or vice versa. Thus, in some cases, instructing the imaging module to capture both in-focus and out-of-focus images of the cells can enhance accuracy of any of the analysis of cells disclosed herein. Alternatively or in addition to, the model(s) can send an instruction to the microfluidic system to modify the flow and adjust an angle of the cell relative to the camera, to adjust focus on different portions of the cell or a subsequent cell. Different portions as disclosed herein can comprise an upper portion, a mid portion, a lower portion, membrane, nucleus, mitochondria, etc. of the cell.

In order to image cells at the right focus (with respect to height or z dimension), what is conventionally done is to calculate the “focus measure” of an image using information theoretic methods like Fourier Transform or Laplace transform.

In some cases, bi-directional out-of-focus (OOF) images cells (e.g., one or more first images that are OOF in a first direction, and one or more second images that are OOF in as second direction that is different—such as opposite—from the first direction). For example, images that are OOF in two opposite directions may be called “bright OOF” image(s) and “dark OOF” image(s), which may be obtained by changing the z-focus bi-directionally. A classifier as disclosed herein can be trained with a image data comprising both bright OOF image(s) and dark OOF image(s). The trained classifiers can be used to run inferences (e.g., in real-time) on new image data of cells to classify each image as bright OOF image, dark OOF image, and optionally image that is not OOF (e.g., not OOF relative to the bright/dark OOF images). The classifier can also measure a percentage of bright OOF image, a percentage of dark OOF image, or a percentage of both bright and dark OOF images within the image data. For example, if any of the percentage of bright OOF image, the percentage of dark OOF image, or the percentage of both bright and dark OOF images is above a threshold value (e.g., a predetermined threshold value), then the classifier can determine that the imaging device (e.g., by the microfluidic system as disclosed herein) may not be imaging cells at the right focal length/plane. The classifier can instruct the user, via GUI of a user device, to adjust the imaging device's focal length/plane. In some examples, the classifier can determine, based on analysis of the image data comprising OOF images, direction and degree of adjustment of focal length/plane that may be required to adjust the imaging device, to yield a reduced amount of OOF imaging. In some examples, the classifier and the microfluidic device can be operatively coupled to a machine learning/artificial intelligence controller, such that the focal length/plane of the imaging device can be adjusted automatically upon determination of the classifier.

A threshold (e.g., a predetermined threshold) of a percentage of OOF images (e.g., bright OOF, dark OOF, or both) can be about 0.1% to about 20%. A threshold (e.g., a predetermined threshold) of a percentage of OOF images (e.g., bright OOF, dark OOF, or both) can be at least about 0.1%. A threshold (e.g., a predetermined threshold) of a percentage of OOF images (e.g., bright OOF, dark OOF, or both) can be at most about 20%. A threshold (e.g., a predetermined threshold) of a percentage of OOF images (e.g., bright OOF, dark OOF, or both) can be about 0.1% to about 0.5%, about 0.1% to about 1%, about 0.1% to about 2%, about 0.1% to about 4%, about 0.1% to about 6%, about 0.1% to about 8%, about 0.1% to about 10%, about 0.1% to about 15%, about 0.1% to about 20%, about 0.5% to about 1%, about 0.5% to about 2%, about 0.5% to about 4%, about 0.5% to about 6%, about 0.5% to about 8%, about 0.5% to about 10%, about 0.5% to about 15%, about 0.5% to about 20%, about 1% to about 2%, about 1% to about 4%, about 1% to about 6%, about 1% to about 8%, about 1% to about 10%, about 1% to about 15%, about 1% to about 20%, about 2% to about 4%, about 2% to about 6%, about 2% to about 8%, about 2% to about 10%, about 2% to about 15%, about 2% to about 20%, about 4% to about 6%, about 4% to about 8%, about 4% to about 10%, about 4% to about 15%, about 4% to about 20%, about 6% to about 8%, about 6% to about 10%, about 6% to about 15%, about 6% to about 20%, about 8% to about 10%, about 8% to about 15%, about 8% to about 20%, about 10% to about 15%, about 10% to about 20%, or about 15% to about 20%. A threshold (e.g., a predetermined threshold) of a percentage of OOF images (e.g., bright OOF, dark OOF, or both) can be about 0.1%, about 0.5%, about 1%, about 2%, about 4%, about 6%, about 8%, about 10%, about 15%, or about 20%.

In some cases, the model(s) can determine that images of different modalities are needed for any of the analysis disclosed herein. Images of varying modalities can comprise a bright field image, a dark field image, a fluorescent image (e.g. of cells stained with a dye), an in-focus image, an out-of-focus image, a greyscale image, a monochrome image, a multi-chrome image, etc.

Any of the models or classifiers disclosed herein can be trained on a set of image data that is annotated with one imaging modality. Alternatively, the models/classifiers can be trained on set of image data that is annotated with a plurality of different imaging modalities (e.g., 2, 3, 4, 5, or more different imaging modalities). Any of the models/classifiers disclosed herein can be trained on a set of image data that is annotated with a spatial coordinate indicative of a position or location within the flow channel. Any of the models/classifiers disclosed herein can be trained on a set of image data that is annotated with a timestamp, such that a set of images can be processed based on the time they are taken.

An image of the image data can be processed in various image processing methods, such as horizontal or vertical image flips, orthogonal rotation, gaussian noise, contrast variation, or noise introduction to mimic microscopic particles or pixel-level aberrations. One or more of the processing methods can be used to generate replicas of the image or analyze the image. In some cases, the image can be processed into a lower-resolution image or a lower-dimension image (e.g., by using one or more deconvolution algorithm).

In any of the methods disclosed herein, processing an image or video from image data can comprise identifying, accounting for, and/or excluding one or more artifacts from the image/video, either automatically or manually by a user. Upon identification, the artifact(s) can be fed into any of the models or classifiers, to train image processing or image analysis. The artifact(s) can be accounted for when classifying the type or state of one or more cells in the image/video. The artifact(s) can be excluded from any determination of the type or state of the cell(s) in the image/video. The artifact(s) can be removed in silico by any of the models/classifiers disclosed herein, and any new replica or modified variant of the image/video excluding the artifact(s) can be stored in a database as disclosed herein. The artifact(s) can be, for example, from debris (e.g., dead cells, dust, etc.), optical conditions during capturing the image/video of the cells (e.g., lighting variability, over-saturation, under-exposure, degradation of the light source, etc.), external factors (e.g., vibrations, misalignment of the microfluidic chip relative to the lighting or optical sensor/camera, power surges/fluctuations, etc.), and changes to the microfluidic system (e.g., deformation/shrinkage/expansion of the microfluidic channel or the microfluidic chip as a whole). The artifacts can be known. The artifacts can be unknown, and the models or classifiers disclosed herein can be configured to define one or more parameters of a new artifact, such that the new artifact can be identified, accounted for, and/or excluded in image processing and analysis.

In some cases, a plurality of artifacts disclosed herein can be identified, accounted for, and/or excluded during image/video processing or analysis. The plurality of artifacts can be weighted the same (e.g., determined to have the same degree of influence on the image/video processing or analysis) or can have different weights (e.g., determined to have different degrees of influence on the image/video processing or analysis). Weight assignments to the plurality of artifacts can be instructed manually by the user or determined automatically by the models/classifiers disclosed herein.

In some cases, one or more reference images or videos of the flow channel (e.g., with or without any cell) can be stored in a database and used as a frame of reference to help identify, account for, and/or exclude any artifact. The reference image(s)/video(s) can be obtained before use of the microfluidic system. The reference image(s)/video(s) can be obtained during the use of the microfluidic system. The reference image(s)/video(s) can be obtained periodically during the use of the microfluidic system, such as, each time the optical sensor/camera captures at least or up to about 5, at least or up to about 10, at least or up to about 20, at least or up to about 50, at least or up to about 100, at least or up to about 200, at least or up to about 500, at least or up to about 1,000, at least or up to about 2,000, at least or up to about 5,000, at least or up to about 10,000, at least or up to about 20,000, at least or up to about 50,000, at least or up to about 100,000 images. The reference image(s)/video(s) can be obtained periodically during the use of the microfluidic system, such as, each time the microfluidic system passes at least or up to about 5, at least or up to about 10, at least or up to about 20, at least or up to about 50, at least or up to about 100, at least or up to about 200, at least or up to about 500, at least or up to about 1,000, at least or up to about 2,000, at least or up to about 5,000, at least or up to about 10,000, at least or up to about 20,000, at least or up to about 50,000, at least or up to about 100,000 cells. The reference image(s)/video(s) can be obtained at landmark periods during the use of the microfluidic system, such as, when the optical sensor/camera captures at least or up to about 5, at least or up to about 10, at least or up to about 20, at least or up to about 50, at least or up to about 100, at least or up to about 200, at least or up to about 500, at least or up to about 1,000, at least or up to about 2,000, at least or up to about 5,000, at least or up to about 10,000, at least or up to about 20,000, at least or up to about 50,000, at least or up to about 100,000 images. The reference image(s)/video(s) can be obtained at landmark periods during the use of the microfluidic system, such as, when the microfluidic system passes at least or up to about 5, at least or up to about 10, at least or up to about 20, at least or up to about 50, at least or up to about 100, at least or up to about 200, at least or up to about 500, at least or up to about 1,000, at least or up to about 2,000, at least or up to about 5,000, at least or up to about 10,000, at least or up to about 20,000, at least or up to about 50,000, at least or up to about 100,000 images.

The method and the platform as disclosed herein can be utilized to process (e.g., modify, analyze, classify) the image data at a rate of about 1,000 images/second to about 100,000,000 images/second. The rate of image data processing can be at least about 1,000 images/second. The rate of image data processing can be at most about 100,000,000 images/second. The rate of image data processing can be about 1,000 images/second to about 5,000 images/second, about 1,000 images/second to about 10,000 images/second, about 1,000 images/second to about 50,000 images/second, about 1,000 images/second to about 100,000 images/second, about 1,000 images/second to about 500,000 images/second, about 1,000 images/second to about 1,000,000 images/second, about 1,000 images/second to about 5,000,000 images/second, about 1,000 images/second to about 10,000,000 images/second, about 1,000 images/second to about 50,000,000 images/second, about 1,000 images/second to about 100,000,000 images/second, about 5,000 images/second to about 10,000 images/second, about 5,000 images/second to about 50,000 images/second, about 5,000 images/second to about 100,000 images/second, about 5,000 images/second to about 500,000 images/second, about 5,000 images/second to about 1,000,000 images/second, about 5,000 images/second to about 5,000,000 images/second, about 5,000 images/second to about 10,000,000 images/second, about 5,000 images/second to about 50,000,000 images/second, about 5,000 images/second to about 100,000,000 images/second, about 10,000 images/second to about 50,000 images/second, about 10,000 images/second to about 100,000 images/second, about 10,000 images/second to about 500,000 images/second, about 10,000 images/second to about 1,000,000 images/second, about 10,000 images/second to about 5,000,000 images/second, about 10,000 images/second to about 10,000,000 images/second, about 10,000 images/second to about 50,000,000 images/second, about 10,000 images/second to about 100,000,000 images/second, about 50,000 images/second to about 100,000 images/second, about 50,000 images/second to about 500,000 images/second, about 50,000 images/second to about 1,000,000 images/second, about 50,000 images/second to about 5,000,000 images/second, about 50,000 images/second to about 10,000,000 images/second, about 50,000 images/second to about 50,000,000 images/second, about 50,000 images/second to about 100,000,000 images/second, about 100,000 images/second to about 500,000 images/second, about 100,000 images/second to about 1,000,000 images/second, about 100,000 images/second to about 5,000,000 images/second, about 100,000 images/second to about 10,000,000 images/second, about 100,000 images/second to about 50,000,000 images/second, about 100,000 images/second to about 100,000,000 images/second, about 500,000 images/second to about 1,000,000 images/second, about 500,000 images/second to about 5,000,000 images/second, about 500,000 images/second to about 10,000,000 images/second, about 500,000 images/second to about 50,000,000 images/second, about 500,000 images/second to about 100,000,000 images/second, about 1,000,000 images/second to about 5,000,000 images/second, about 1,000,000 images/second to about 10,000,000 images/second, about 1,000,000 images/second to about 50,000,000 images/second, about 1,000,000 images/second to about 100,000,000 images/second, about 5,000,000 images/second to about 10,000,000 images/second, about 5,000,000 images/second to about 50,000,000 images/second, about 5,000,000 images/second to about 100,000,000 images/second, about 10,000,000 images/second to about 50,000,000 images/second, about 10,000,000 images/second to about 100,000,000 images/second, or about 50,000,000 images/second to about 100,000,000 images/second. The rate of image data processing can be about 1,000 images/second, about 5,000 images/second, about 10,000 images/second, about 50,000 images/second, about 100,000 images/second, about 500,000 images/second, about 1,000,000 images/second, about 5,000,000 images/second, about 10,000,000 images/second, about 50,000,000 images/second, or about 100,000,000 images/second.

The method and the platform as disclosed herein can be utilized to process (e.g., modify, analyze, classify) the image data at a rate of about 1,000 cells/second to about 100,000,000 cells/second. The rate of image data processing can be at least about 1,000 cells/second. The rate of image data processing can be at most about 100,000,000 cells/second. The rate of image data processing can be about 1,000 cells/second to about 5,000 cells/second, about 1,000 cells/second to about 10,000 cells/second, about 1,000 cells/second to about 50,000 cells/second, about 1,000 cells/second to about 100,000 cells/second, about 1,000 cells/second to about 500,000 cells/second, about 1,000 cells/second to about 1,000,000 cells/second, about 1,000 cells/second to about 5,000,000 cells/second, about 1,000 cells/second to about 10,000,000 cells/second, about 1,000 cells/second to about 50,000,000 cells/second, about 1,000 cells/second to about 100,000,000 cells/second, about 5,000 cells/second to about 10,000 cells/second, about 5,000 cells/second to about 50,000 cells/second, about 5,000 cells/second to about 100,000 cells/second, about 5,000 cells/second to about 500,000 cells/second, about 5,000 cells/second to about 1,000,000 cells/second, about 5,000 cells/second to about 5,000,000 cells/second, about 5,000 cells/second to about 10,000,000 cells/second, about 5,000 cells/second to about 50,000,000 cells/second, about 5,000 cells/second to about 100,000,000 cells/second, about 10,000 cells/second to about 50,000 cells/second, about 10,000 cells/second to about 100,000 cells/second, about 10,000 cells/second to about 500,000 cells/second, about 10,000 cells/second to about 1,000,000 cells/second, about 10,000 cells/second to about 5,000,000 cells/second, about 10,000 cells/second to about 10,000,000 cells/second, about 10,000 cells/second to about 50,000,000 cells/second, about 10,000 cells/second to about 100,000,000 cells/second, about 50,000 cells/second to about 100,000 cells/second, about 50,000 cells/second to about 500,000 cells/second, about 50,000 cells/second to about 1,000,000 cells/second, about 50,000 cells/second to about 5,000,000 cells/second, about 50,000 cells/second to about 10,000,000 cells/second, about 50,000 cells/second to about 50,000,000 cells/second, about 50,000 cells/second to about 100,000,000 cells/second, about 100,000 cells/second to about 500,000 cells/second, about 100,000 cells/second to about 1,000,000 cells/second, about 100,000 cells/second to about 5,000,000 cells/second, about 100,000 cells/second to about 10,000,000 cells/second, about 100,000 cells/second to about 50,000,000 cells/second, about 100,000 cells/second to about 100,000,000 cells/second, about 500,000 cells/second to about 1,000,000 cells/second, about 500,000 cells/second to about 5,000,000 cells/second, about 500,000 cells/second to about 10,000,000 cells/second, about 500,000 cells/second to about 50,000,000 cells/second, about 500,000 cells/second to about 100,000,000 cells/second, about 1,000,000 cells/second to about 5,000,000 cells/second, about 1,000,000 cells/second to about 10,000,000 cells/second, about 1,000,000 cells/second to about 50,000,000 cells/second, about 1,000,000 cells/second to about 100,000,000 cells/second, about 5,000,000 cells/second to about 10,000,000 cells/second, about 5,000,000 cells/second to about 50,000,000 cells/second, about 5,000,000 cells/second to about 100,000,000 cells/second, about 10,000,000 cells/second to about 50,000,000 cells/second, about 10,000,000 cells/second to about 100,000,000 cells/second, or about 50,000,000 cells/second to about 100,000,000 cells/second. The rate of image data processing can be about 1,000 cells/second, about 5,000 cells/second, about 10,000 cells/second, about 50,000 cells/second, about 100,000 cells/second, about 500,000 cells/second, about 1,000,000 cells/second, about 5,000,000 cells/second, about 10,000,000 cells/second, about 50,000,000 cells/second, or about 100,000,000 cells/second.

The method and the platform as disclosed herein can be utilized to process (e.g., modify, analyze, classify) the image data at a rate of about 1,000 datapoints/second to about 100,000,000 datapoints/second. The rate of image data processing can be at least about 1,000 datapoints/second. The rate of image data processing can be at most about 100,000,000 datapoints/second. The rate of image data processing can be about 1,000 datapoints/second to about 5,000 datapoints/second, about 1,000 datapoints/second to about 10,000 datapoints/second, about 1,000 datapoints/second to about 50,000 datapoints/second, about 1,000 datapoints/second to about 100,000 datapoints/second, about 1,000 datapoints/second to about 500,000 datapoints/second, about 1,000 datapoints/second to about 1,000,000 datapoints/second, about 1,000 datapoints/second to about 5,000,000 datapoints/second, about 1,000 datapoints/second to about 10,000,000 datapoints/second, about 1,000 datapoints/second to about 50,000,000 datapoints/second, about 1,000 datapoints/second to about 100,000,000 datapoints/second, about 5,000 datapoints/second to about 10,000 datapoints/second, about 5,000 datapoints/second to about 50,000 datapoints/second, about 5,000 datapoints/second to about 100,000 datapoints/second, about 5,000 datapoints/second to about 500,000 datapoints/second, about 5,000 datapoints/second to about 1,000,000 datapoints/second, about 5,000 datapoints/second to about 5,000,000 datapoints/second, about 5,000 datapoints/second to about 10,000,000 datapoints/second, about 5,000 datapoints/second to about 50,000,000 datapoints/second, about 5,000 datapoints/second to about 100,000,000 datapoints/second, about 10,000 datapoints/second to about 50,000 datapoints/second, about 10,000 datapoints/second to about 100,000 datapoints/second, about 10,000 datapoints/second to about 500,000 datapoints/second, about 10,000 datapoints/second to about 1,000,000 datapoints/second, about 10,000 datapoints/second to about 5,000,000 datapoints/second, about 10,000 datapoints/second to about 10,000,000 datapoints/second, about 10,000 datapoints/second to about 50,000,000 datapoints/second, about 10,000 datapoints/second to about 100,000,000 datapoints/second, about 50,000 datapoints/second to about 100,000 datapoints/second, about 50,000 datapoints/second to about 500,000 datapoints/second, about 50,000 datapoints/second to about 1,000,000 datapoints/second, about 50,000 datapoints/second to about 5,000,000 datapoints/second, about 50,000 datapoints/second to about 10,000,000 datapoints/second, about 50,000 datapoints/second to about 50,000,000 datapoints/second, about 50,000 datapoints/second to about 100,000,000 datapoints/second, about 100,000 datapoints/second to about 500,000 datapoints/second, about 100,000 datapoints/second to about 1,000,000 datapoints/second, about 100,000 datapoints/second to about 5,000,000 datapoints/second, about 100,000 datapoints/second to about 10,000,000 datapoints/second, about 100,000 datapoints/second to about 50,000,000 datapoints/second, about 100,000 datapoints/second to about 100,000,000 datapoints/second, about 500,000 datapoints/second to about 1,000,000 datapoints/second, about 500,000 datapoints/second to about 5,000,000 datapoints/second, about 500,000 datapoints/second to about 10,000,000 datapoints/second, about 500,000 datapoints/second to about 50,000,000 datapoints/second, about 500,000 datapoints/second to about 100,000,000 datapoints/second, about 1,000,000 datapoints/second to about 5,000,000 datapoints/second, about 1,000,000 datapoints/second to about 10,000,000 datapoints/second, about 1,000,000 datapoints/second to about 50,000,000 datapoints/second, about 1,000,000 datapoints/second to about 100,000,000 datapoints/second, about 5,000,000 datapoints/second to about 10,000,000 datapoints/second, about 5,000,000 datapoints/second to about 50,000,000 datapoints/second, about 5,000,000 datapoints/second to about 100,000,000 datapoints/second, about 10,000,000 datapoints/second to about 50,000,000 datapoints/second, about 10,000,000 datapoints/second to about 100,000,000 datapoints/second, or about 50,000,000 datapoints/second to about 100,000,000 datapoints/second. The rate of image data processing can be about 1,000 datapoints/second, about 5,000 datapoints/second, about 10,000 datapoints/second, about 50,000 datapoints/second, about 100,000 datapoints/second, about 500,000 datapoints/second, about 1,000,000 datapoints/second, about 5,000,000 datapoints/second, about 10,000,000 datapoints/second, about 50,000,000 datapoints/second, or about 100,000,000 datapoints/second.

Any of the methods or platforms disclosed herein can be operatively coupled to an online crowdsourcing platform. The online crowdsourcing platform can comprise any of the database disclosed herein. For example, the database can store a plurality of single cell images that are grouped into morphologically-distinct clusters corresponding to a plurality of cell classes (e.g., predetermined cell types or states). The online crowdsourcing platform can comprise one or more models or classifiers as disclosed herein (e.g., a modeling library comprising one or more machine learning models/classifiers as disclosed herein). The online crowdsourcing platform can comprise a web portal for a community of users to share contents, e.g., (1) upload, download, search, curate, annotate, or edit one or more existing images or new images into the database, (2) train or validate the one or more model(s)/classifier(s) using datasets from the database, and/or (3) upload new models into the modeling library. In some cases, the online crowdsourcing platform can allow users to buy, sell, share, or exchange the model(s)/classifier(s) with one another.

In some cases, the web portal can be configured to generate incentives for the users to update the database with new annotated cell images, model(s), and/or classifier(s). Incentives may be monetary. Incentives may be additional access to the global CMA, model(s), and/or classifier(s). In some cases, the web portal can be configured to generate incentives for the users to download, use, and review (e.g., rate or leave comments) any of the annotated cell images, model(s), and/or classifier(s) from, e.g., other users.

In some cases, a global cell morphology atlas (global CMA) can be generated by collecting (i) annotated cell images, (ii) cell morphology maps or ontologies, (iii), and/or (iv) classifiers from the users via the web portal. The global CMA can then be shared with the users via the web portal. All users can have access to the global CMA. Alternatively, specifically defined users can have access to specifically defined portions of the global CMA. For example, cancer centers can have access to “cancer cells” portion of the global CMA, e.g., via a subscription based service. In a similar fashion, global models or classifiers may be generated based on the annotated cell images, model(s), and/or classifiers that are collected from the users via the web portal.

III. Additional Aspects of Cell Analysis

Any of the systems and methods disclosed can be utilized to sort the cell. A cell may be directed through a flow channel, and one or more imaging devices (e.g., sensor(s), camera(s)) can be configured to capture one or more images/videos of the cell passing through. Subsequently, the image(s)/video(s) of the cell can be analyzed as disclosed herein (e.g., by the classifier to plot the cell as a datapoint in a cell morphology map, determine a most likely cluster it belongs to, and determine a final classification of the cell based on the selected cluster) in real-time, such that a decision can be made in real-time (e.g., automatically by the machine learning algorithm) to determine (i) whether to sort the cell or not and/or (ii) which sub-channel of a plurality of sub-channels to sort the cell into.

Any of the systems and methods disclosed herein can be processed or performed (e.g., automatically) in real-time. The term “real time” or “real-time,” as used interchangeably herein, generally refers to an event (e.g., an operation, a process, a method, a technique, a computation, a calculation, an analysis, an optimization, etc.) that is performed using recently obtained (e.g., collected or received) data. Examples of the event may include, but are not limited to, analysis of a one or more images of a cell to classify the cell, updating one or more deep learning algorithms (e.g., neural networks) for classification and sorting, controlling one or more process within the flow channel (e.g., actuation of one or more valves by at a sorting bifurcation, etc.) based on any analysis of the imaging of cells or the flow channel, etc. In some cases, a real time event may be performed almost immediately or within a short enough time span, such as within at least 0.0001 ms, 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

The cell sorting system as disclosed herein can comprise a flow channel configured to transport a cell through the channel. The cell sorting system can comprise an imaging device configured to capture an image of the cell from a plurality of different angles as the cell is transported through the flow channel. The cell sorting system can comprise a processor configured to analyze the image using a deep learning algorithm to enable sorting of the cell. The cell sorting system can be a cell classification system. In some cases, the flow channel can be configured to transport a solvent (e.g., liquid, water, media, alcohol, etc.) without any cell. The cell sorting system can have one or more mechanisms (e.g., a motor) for moving the imaging device relative to the channel. Such movement can be relative movement, and thus the moving piece can be the imaging device, the channel, or both. The processor can be further configured to control such relative movement.

Any of the systems and methods disclosed herein can be utilized to enrich a target cell or a target population of cells, e.g., without any cell labeling. As used herein, the term “enrichment” refers to a change in relative proportion (e.g., percentage) of at least one species (e.g., one type of cell of interest) in a pool of multiple species (e.g., a pool of multiple types of cells), in which a proportion of the at least one species increases relative to one or more other species from the pool of multiple species. In some cases, the systems and methods of the present disclosure can be utilized to effect enrichment of a cell type of interest (e.g., a diseased cell, a cancer cell, a healthy cell, etc.) in a pool of multiple cell types by at least about 0.1-fold, at least about 0.2-fold, at least about 0.5-fold, at least about 0.8-fold, at least about 1-fold, at least about 2-fold, at least about 5-fold, at least about 8-fold, at least about 10-fold, at least about 20-fold, at least about 50-fold, at least about 80-fold, at least about 100-fold, at least about 200-fold, at least about 500-fold, at least about 800-fold, at least about 1,000-fold, at least about 2,000-fold, at least about 5,000-fold, at least about 8,000-fold, at least about 10,000-fold, at least about 20,000-fold, at least about 50,000-fold, at least about 80,000-fold, at least about 100,000-fold, at least about 200,000-fold, at least about 500,000-fold, at least about 800,000-fold, at least about 1,000,000-fold, or higher, as compared to a proportion of another cell type in the pool.

Without wishing to be bound by theory, the sorting or enrichment of one or more cells as disclosed herein (e.g., via cell morphology-based classification) can effect sorting or enrichment or cells exhibiting (i) a nucleic acid composition of interest, (ii) transcriptome composition of interest, and/or (ii) a protein expression profile of interest. In some cases, any one of (i), (ii), and (iii) can result in a cell with a specific morphology (e.g., a neuronal gene expression profile leading to a neuronal cell-like morphology, a cancer gene expression profile leader to a cancer cell-like morphology, a stemness gene expression profile leading to a stem cell-like morphology, etc.), and thus cell sorting or enrichment via cell morphology can indirectly sort or enrich cells exhibiting any one of (i), (ii), and (iii).

Any of the systems and methods disclosed herein can be utilized to generate a sorted or enriched sample of a cell type of interest, and a purity of such sample with respect to a proportion of the cell type of interest can be at least about 70%, at least about 72%, at least about 75%, at least about 80%, at least about 82%, at least about 85%, at least about 90%, at least about 91%, at least about 92%, at least about 93%, at least about 94%, at least about 95%, at least about 96%, at least about 97%, at least about 98%, at least about 99%, or about 100%.

In some embodiments of any the systems and methods disclosed herein can, a cell that is sorted or enriched may not arise from mitosis subsequent to or during the sorting or enrichment. For example, the cell that is sorted or enriched may be found in an original population of cells that is subjected to the sorting or enrichment.

some embodiments of any the systems and methods disclosed herein can, the sorting or enrichment of a cell from a pool of cells may not substantially change one or more characteristics (e.g., expression or activity level of one or more genes, such as endogenous genes) of the cell. For example, the cell sorting or enrichment may not substantially change (e.g., decrease and/or increase) expression or activity level of a gene of interest in the cell. In another example, the cell sorting or enrichment may not substantially change transcriptional profile of the cell. In some cases, upon the cell sorting or enrichment as disclosed herein, a degree of change of one or more characteristics of the cell (e.g., as compared to that prior to the cell sorting or enrichment, or as compared to a control cell that is not subjected to the cell sorting or enrichment) may be less than or equal to about 20%, less than or equal to about 19%, less than or equal to about 18%, less than or equal to about 17%, less than or equal to about 16%, less than or equal to about 15%, less than or equal to about 14%, less than or equal to about 13%, less than or equal to about 12%, less than or equal to about 11%, less than or equal to about 10%, less than or equal to about 9%, less than or equal to about 8%, less than or equal to about 7%, less than or equal to about 6%, less than or equal to about 5%, less than or equal to about 4%, less than or equal to about 3%, less than or equal to about 2%, less than or equal to about 1%, less than or equal to about 0.9%, less than or equal to about 0.8%, less than or equal to about 0.7%, less than or equal to about 0.6%, less than or equal to about 0.5%, less than or equal to about 0.4%, less than or equal to about 0.3%, less than or equal to about 0.2%, or less than or equal to about 0.1.

### Microfluidic Systems and Methods Thereof

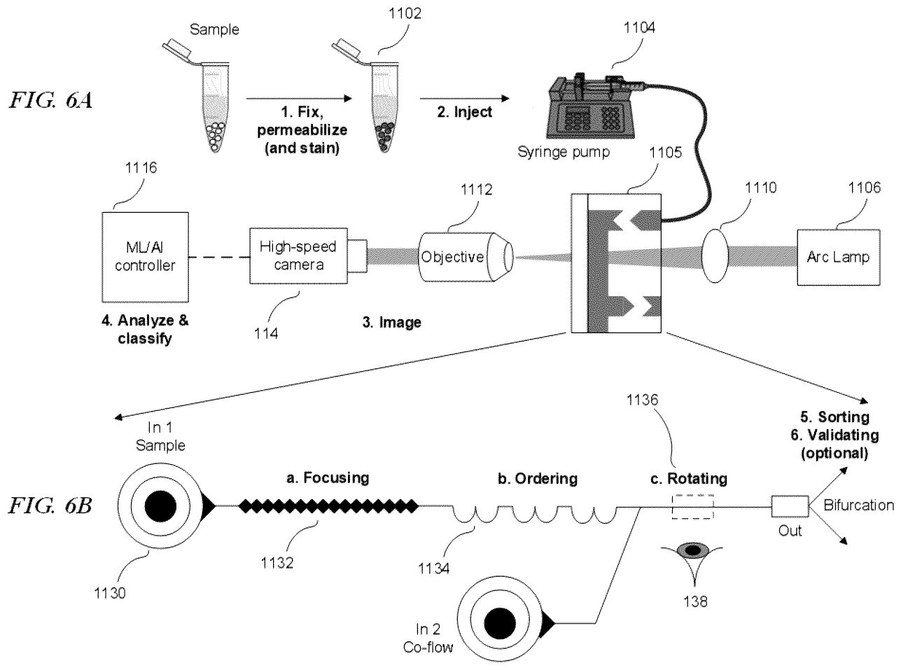

FIG. 6A shows a schematic illustration of the cell sorting system, as disclosed herein, with a flow cell design (e.g., a microfluidic design), with further details illustrated in FIG. 6B. The cell sorting system can be operatively coupled to a machine learning or artificial intelligence controller. Such ML/AI controller can be configured to perform any of the methods disclosed herein. Such ML/AI controller can be operatively coupled to any of the platforms disclosed herein.

In operation, a sample 1102 is prepared and injected by a pump 1104 (e.g., a syringe pump) into a flow cell 1105, or flow-through device. In some embodiments, the flow cell 1105 is a microfluidic device. Although FIG. 6A illustrates a classification and/or sorting system utilizing a syringe pump, any of a number of perfusion systems can be used such as (but not limited to) gravity feeds, peristalsis, or any of a number of pressure systems. In some embodiments, the sample is prepared by fixation and staining. In some examples, the sample comprises live cells. As can readily be appreciated, the specific manner in which the sample is prepared is largely dependent upon the requirements of a specific application.

Examples of the flow unit may be, but are not limited to, a syringe pump, a vacuum pump, an actuator (e.g., linear, pneumatic, hydraulic, etc.), a compressor, or any other suitable device to exert pressure (positive, negative, alternating thereof, etc.) to a fluid that may or may not comprise one or more particles (e.g., one or more cells to be classified, sorted, and/or analyzed). The flow unit may be configured to raise, compress, move, and/or transfer fluid into or away from the microfluidic channel. In some examples, the flow unit may be configured to deliver positive pressure, alternating positive pressure and vacuum pressure, negative pressure, alternating negative pressure and vacuum pressure, and/or only vacuum pressure. The flow cell of the present disclosure may comprise at least 1, 2, 3, 4, 5, 6, 7, 8, 9, 10, or more flow units. The flow cell may comprise at most 10, 9, 8, 7, 6, 5, 4, 3, 2, or 1 flow unit.

Each flow unit may be in fluid communication with at least 1, 2, 3, 4, 5, 6, 7, 8, 9, 10, or more sources of fluid. Each flow unit may be in fluid communication with at most 10, 9, 8, 7, 6, 5, 4, 3, 2, or 1 fluid. The fluid may contain the particles (e.g., cells). Alternatively, the fluid may be particle-free. The flow unit may be configured to maintain, increase, and/or decrease a flow velocity of the fluid within the microfluidic channel of the flow unit. Thus, the flow unit may be configured to maintain, increase, and/or decrease a flow velocity (e.g., downstream of the microfluidic channel) of the particles. The flow unit may be configured to accelerate or decelerate a flow velocity of the fluid within the microfluidic channel of the flow unit, thereby accelerating or decelerating a flow velocity of the particles.

The fluid may be liquid or gas (e.g., air, argon, nitrogen, etc.). The liquid may be an aqueous solution (e.g., water, buffer, saline, etc.). Alternatively, the liquid may be oil. In some cases, only one or more aqueous solutions may be directed through the microfluidic channels. Alternatively, only one or more oils may be directed through the microfluidic channels. In another alternative, both aqueous solution(s) and oil(s) may be directed through the microfluidic channels. In some examples, (i) the aqueous solution may form droplets (e.g., emulsions containing the particles) that are suspended in the oil, or (ii) the oil may form droplets (e.g., emulsions containing the particles) that are suspended in the aqueous solution.

As can readily be appreciated, any perfusion system, including but not limited to peristalsis systems and gravity feeds, appropriate to a given classification and/or sorting system can be utilized.

As noted above, the flow cell 1105 can be implemented as a fluidic device that focuses cells from the sample into a single streamline that is imaged continuously. In the illustrated embodiment, the cell line is illuminated by a light source 1106 (e.g., a lamp, such as an arc lamp) and an optical system 1110 that directs light onto an imaging region 1138 of the flow cell 1105. An objective lens system 1112 magnifies the cells by directing light toward the sensor of a high-speed camera system 114.

In some embodiments, a 10×, 20×, 40×, 60×, 80×, 100×, or 200× objective is used to magnify the cells. In some embodiments, a 10×, objective is used to magnify the cells. In some embodiments, a 20× objective is used to magnify the cells. In some embodiments, a 40× objective is used to magnify the cells. In some embodiments, a 60× objective is used to magnify the cells. In some embodiments, a 80× objective is used to magnify the cells. In some embodiments, a 100× objective is used to magnify the cells. In some embodiments, a 200× objective is used to magnify the cells. In some embodiments, a 10× to a 200× objective is used to magnify the cells, for example a 10×-20×, a 10×-40×, a 10×-60×, a 10×-80×, or al Ox-100× objective is used to magnify the cells.

As can readily be appreciated by a person having ordinary skill in the art, the specific magnification utilized can vary greatly and is largely dependent upon the requirements of a given imaging system and cell types of interest.

In some embodiments, one or more imaging devices may be used to capture images of the cell. In some aspects, the imaging device is a high-speed camera. In some aspects, the imaging device is a high-speed camera with a micro-second exposure time. In some instances, the exposure time is 1 millisecond. In some instances, the exposure time is between 1 millisecond (ms) and 0.75 millisecond. In some instances, the exposure time is between 1 ms and 0.50 ms. In some instances, the exposure time is between 1 ms and 0.25 ms. In some instances, the exposure time is between 0.75 ms and 0.50 ms. In some instances, the exposure time is between 0.75 ms and 0.25 ms. In some instances, the exposure time is between 0.50 ms and 0.25 ms. In some instances, the exposure time is between 0.25 ms and 0.1 ms. In some instances, the exposure time is between 0.1 ms and 0.01 ms. In some instances, the exposure time is between 0.1 ms and 0.001 ms. In some instances, the exposure time is between 0.1 ms and 1 microsecond (μs). In some aspects, the exposure time is between 1 μs and 0.1 μs. In some aspects, the exposure time is between 1 μs and 0.01 μs. In some aspects, the exposure time is between 0.1 μs and 0.01 μs. In some aspects, the exposure time is between 1 μs and 0.001 μs. In some aspects, the exposure time is between 0.1 μs and 0.001 μs. In some aspects, the exposure time is between 0.01 μs and 0.001 μs.

In some cases, the flow cell 1105 may comprise at least 1, 2, 3, 4, 5, 6, 7, 8, 9, 10, or more imaging devices (e.g., the high-speed camera system 114) on or adjacent to the imaging region 1138. In some cases, the flow cell may comprise at most 10, 9, 8, 7, 6, 5, 4, 3, 2, or 1 imaging device on or adjacent to the imaging region 1138. In some cases, the flow cell 1105 may comprise a plurality of imaging devices. Each of the plurality of imaging devices may use light from a same light source. Alternatively, each of the plurality of imaging devices may use light from different light sources. The plurality of imaging devices may be configured in parallel and/or in series with respect to one another. The plurality of imaging devices may be configured on one or more sides (e.g., two adjacent sides or two opposite sides) of the flow cell 1105. The plurality of imaging devices may be configured to view the imaging region 1138 along a same axis or different axes with respect to (i) a length of the flow cell 1105 (e.g., a length of a straight channel of the flow cell 1105) or (ii) a direction of migration of one or more particles (e.g., one or more cells) in the flow cell 1105.

One or more imaging devices of the present disclosure may be stationary while imaging one or more cells, e.g., at the imaging region 1138. Alternatively, one or more imaging devices may move with respect to the flow channel (e.g., along the length of the flow channel, towards and/or away from the flow channel, tangentially about the circumference of the flow channel, etc.) while imaging the one or more cells. In some examples, the one or more imaging devices may be operatively coupled to one or more actuators, such as, for example, a stepper actuator, linear actuator, hydraulic actuator, pneumatic actuator, electric actuator, magnetic actuator, and mechanical actuator (e.g., rack and pinion, chains, etc.).

In some cases, the flow cell 1105 may comprise at least 1, 2, 3, 4, 5, 6, 7, 8, 9, 10, or more imaging regions (e.g., the imaging region 1138). In some cases, the flow cell 1105 may comprise at most 10, 9, 8, 7, 6, 5, 4, 3, 2, or 1 imaging region. In some examples, the flow cell 1115 may comprise a plurality of imaging regions, and the plurality of imaging regions may be configured in parallel and/or in series with respect to each another. The plurality of imaging regions may or may not be in fluid communication with each other. In an example, a first imaging region and a second imaging region may be configured in parallel, such that a first fluid that passes through the first imaging region does not pass through a second imaging region. In another example, a first imaging region and a second imaging region may be configured in series, such that a first fluid that passes through the first imaging region also passes through the second imaging region.

The imaging device(s) (e.g., the high-speed camera) of the imaging system can comprise an electromagnetic radiation sensor (e.g., IR sensor, color sensor, etc.) that detects at least a portion of the electromagnetic radiation that is reflected by and/or transmitted from the flow cell or any content (e.g., the cell) in the flow cell. The imaging device can be in operative communication with one or more sources (e.g., at least 1, 2, 3, 4, 5, or more) of the electromagnetic radiation. The electromagnetic radiation can comprise one or more wavelengths from the electromagnetic spectrum including, but not limited to x-rays (about 0.1 nanometers (nm) to about 10.0 nm; or about 10¹⁸ Hertz (Hz) to about 10¹⁶ Hz), ultraviolet (UV) rays (about 10.0 nm to about 380 nm; or about 8×10¹⁶ Hz to about 10¹⁵ Hz), visible light (about 380 nm to about 750 nm; or about 8×10¹⁴ Hz to about 4×10¹⁴ Hz), infrared (IR) light (about 750 nm to about 0.1 centimeters (cm); or about 4×10¹⁴ Hz to about 5×10¹¹ Hz), and microwaves (about 0.1 cm to about 100 cm; or about 10⁸ Hz to about 5×10¹¹ Hz). In some cases, the source(s) of the electromagnetic radiation can be ambient light, and thus the cell sorting system may not have an additional source of the electromagnetic radiation.

The imaging device(s) can be configured to take a two-dimensional image (e.g., one or more pixels) of the cell and/or a three-dimensional image (e.g., one or more voxels) of the cell.

As can readily be appreciated, the exposure times can differ across different systems and can largely be dependent upon the requirements of a given application or the limitations of a given system such as but not limited to flow rates. Images are acquired and can be analyzed using an image analysis algorithm.

In some embodiments, the images are acquired and analyzed post-capture. In some aspects, the images are acquired and analyzed in real-time continuously. Using object tracking software, single cells can be detected and tracked while in the field of view of the camera. Background subtraction can then be performed. In a number of embodiments, the flow cell 1106 causes the cells to rotate as they are imaged, and multiple images of each cell are provided to a computing system 1116 for analysis. In some embodiments, the multiple images comprise images from a plurality of cell angles.

The flow rate and channel dimensions can be determined to obtain multiple images of the same cell from a plurality of different angles (i.e., a plurality of cell angles). A degree of rotation between an angle to the next angle may be uniform or non-uniform. In some examples, a full 360° view of the cell is captured. In some embodiments, 4 images are provided in which the cell rotates 90° between successive frames. In some embodiments, 8 images are provided in which the cell rotates 45° between successive frames. In some embodiments, 24 images are provided in which the cell rotates 15° between successive frames. In some embodiments, at least three or more images are provided in which the cell rotates at a first angle between a first frame and a second frame, and the cell rotates at a second angle between the second frame and a third frame, wherein the first and second angles are different. In some examples, less than the full 360° view of the cell may be captured, and a resulting plurality of images of the same cell may be sufficient to classify the cell (e.g., determine a specific type of the cell).

The cell can have a plurality of sides. The plurality of sides of the cell can be defined with respect to a direction of the transport (flow) of the cell through the channel. In some cases, the cell can comprise a stop side, a bottom side that is opposite the top side, a front side (e.g., the side towards the direction of the flow of the cell), a rear side opposite the front side, a left side, and/or a right side opposite the left side. In some cases, the image of the cell can comprise a plurality of images captured from the plurality of angles, wherein the plurality of images comprise: (1) an image captured from the top side of the cell, (2) an image captured from the bottom side of the cell, (3) an image captured from the front side of the cell, (4) an image captured from the rear side of the cell, (5) an image captured from the left side of the cell, and/or (6) an image captured from the right side of the cell.

In some embodiments, a two-dimensional “hologram” of a cell can be generated by superimposing the multiple images of the individual cell. The “hologram” can be analyzed to automatically classify characteristics of the cell based upon features including but not limited to the morphological features of the cell.

In some embodiments, 1, 2, 3, 4, 5, 6, 7, 8, 9, or 10 images are captured for each cell. In some embodiments, 5 or more images are captured for each cell. In some embodiments, from 5 to 10 images are captured for each cell. In some embodiments, 10 or more images are captured for each cell. In some embodiments, from 10 to 20 images are captured for each cell. In some embodiments, 20 or more images are captured for each cell. In some embodiments, from 20 to 50 images are captured for each cell. In some embodiments, 50 or more images are captured for each cell. In some embodiments, from 50 to 100 images are captured for each cell. In some embodiments, 100 or more images are captured for each cell. In some cases, at least 1, 2, 3, 4, 5, 6, 7, 8, 9, 10, 15, 20, 30, 40, 50, or more images may be captured for each cell at a plurality of different angles. In some cases, at most 50, 40, 30, 20, 15, 10, 9, 8, 7, 6, 5, 4, 3, or 2 images may be captured for each cell at a plurality of different angles.

In some embodiments, the imaging device is moved so as to capture multiple images of the cell from a plurality of angles. In some aspects, the images are captured at an angle between 0 and 90 degrees to the horizontal axis. In some aspects, the images are captured at an angle between 90 and 180 degrees to the horizontal axis. In some aspects, the images are captured at an angle between 180 and 270 degrees to the horizontal axis. In some aspects, the images are captured at an angle between 270 and 360 degrees to the horizontal axis.

In some embodiments, multiple imaging devices (for e.g. multiple cameras) are used wherein each device captures an image of the cell from a specific cell angle. In some aspects, 2, 3, 4, 5, 6, 7, 8, 9, or 10 cameras are used. In some aspects, more than 10 cameras are used, wherein each camera images the cell from a specific cell angle,

As can readily be appreciated, the number of images that are captured is dependent upon the requirements of a given application or the limitations of a given system. In several embodiments, the flow cell has different regions to focus, order, and/or rotate cells. Although the focusing regions, ordering regions, and cell rotating regions are discussed as affecting the sample in a specific sequence, a person having ordinary skill in the art would appreciate that the various regions can be arranged differently, where the focusing, ordering, and/or rotating of the cells in the sample can be performed in any order. Regions within a microfluidic device implemented in accordance with an embodiment of the disclosure are illustrated in FIG. 6B. Flow cell 1105 may include a filtration region 1130 to prevent channel clogging by aggregates/debris or dust particles. Cells pass through a focusing region 1132 that focuses the cells into a single streamline of cells that are then spaced by an ordering region 1134. In some embodiments, the focusing region utilizes “inertial focusing” to form the single streamline of cells. In some embodiments, the focusing region utilizes ‘hydrodynamic focusing” to focus the cells into the single streamline of cells. Optionally, prior to imaging, rotation can be imparted upon the cells by a rotation region 1136. The optionally spinning cells can then pass through an imaging region 1138 in which the cells are illuminated for imaging prior to exiting the flow cell. These various regions are described and discussed in further detail below. In some cases, the rotation region 1136 may precede the imaging region 1138. In some cases, the rotation region 1136 may be a part (e.g., a beginning portion, a middle portion, and/or an end portion with respect to a migration of a cell within the flow cell) of the imaging region 1138. In some cases, the imaging region 1138 may be a part of the rotation region 1136.

In some embodiments, a single cell is imaged in a field of view of the imaging device, e.g. camera. In some embodiments, multiple cells are imaged in the same field of view of the imaging device. In some aspects, 1, 2, 3, 4, 5, 6, 7, 8, 9, or 10 cells are imaged in the same field of view of the imaging device. In some aspects, up to 100 cells are imaged in the same field of view of the imaging device. In some instances, 10 to 100 cells are imaged in the field of view, for example, 10 to 20 cells, 10 to 30 cells, 10 to 40 cells, 10 to 50 cells, 10 to 60 cells, 10 to 80 cells, 10 to 90 cells, 20 to 30 cells, 20 to 40 cells, 20 to 50 cells, 20 to 60 cells, 20 to 70 cells, 20 to 80 cells, 20 to 90 cells, 30 to 40 cells, 40 to 50 cells, 40 to 60 cells, 40 to 70 cells, 40 to 80 cells, 40 to 90 cells, 50 to 60 cells, 50 to 70 cells, 50 to 80 cells, 50 to 90 cells, 60 to 70 cells, 60 to 80 cells, 60 to 90 cells, 70 to 80 cells, 70 to 90 cells, 90 to 100 cells are imaged in the same field of view of the imaging device.

In some cases, only a single cell may be allowed to be transported across a cross-section of the flow channel perpendicular to the axis of the flow channel. In some cases, a plurality of cells (e.g., at least 2, 3, 4, 5, or more cells; at most 5, 4, 3, 2, or 1 cell) may be allowed to be transported simultaneously across the cross-section of the flow channel perpendicular to the axis of the flow channel. In such a case, the imaging device (or the processor operatively linked to the imaging device) may be configured to track each of the plurality of cells as they are transported along the flow channel.

The imaging system can include, among other things, a camera, an objective lens system and a light source. In a number of embodiments, flow cells similar to those described above can be fabricated using standard 2D microfluidic fabrication techniques, requiring minimal fabrication time and cost.

Although specific classification and/or sorting systems, flow cells, and microfluidic devices are described above with respect to FIGS. 6A and 6B, classification and/or sorting systems can be implemented in any of a variety of ways appropriate to the requirements of specific applications in accordance with various embodiments of the disclosure. Specific elements of microfluidic devices that can be utilized in classification and/or sorting systems in accordance with some embodiments of the disclosure are discussed further below.

In some cases, embodiments, the microfluidic system can comprise a microfluidic chip (e.g., comprising one or more microfluidic channels for flowing cells) operatively coupled to an imaging device (e.g., one or more cameras). A microfluidic device can comprise the imaging device, and the chip can be inserted into the device, to align the imaging device to an imaging region of a channel of the chip. To align the chip to the precise location for the imaging, the chip can comprise one or more positioning identifiers (e.g., pattern(s), such as numbers, letters, symbols, or other drawings) that can be imaged to determine the positioning of the chip (and thus the imaging region of the channel of the chip) relative to the device as a whole or relative to the imaging device. For image-based alignment (e.g., auto-alignment) of the chip within the device, one or more images of the chip can be capture upon its coupling to the device, and the image(s) can be analyzed by any of the methods disclosed herein (e.g., using any model or classifier disclosed herein) to determine a degree or score of chip alignment. The positioning identifier(s) can be a “guide” to navigate the stage holding the chip within the device to move within the device towards a correct position relative to the imaging unit.

In some cases, rule-based image processing can be used to navigate the stage to a precise range of location or a precise location relative to the image unit.

In some cases, machine learning/artificial intelligence methods as disclosed herein can be modified or trained to identify the pattern on the chip and navigate the stage to the precise imaging location for the image unit, to increase resilience.

In some cases, machine learning/artificial intelligence methods as disclosed herein can be modified or trained to implement reinforcement learning based alignment and focusing. The alignment process for the chip to the instrument or the image unit can involve moving the stage holding the chip in, e.g., either X or Y axis and/or moving the imaging plane on the Z axis. In the training process, (i) the chip can start at a X, Y, and Z position (e.g., randomly selected), (ii) based on one or more image(s) of the chip and/or the stage holding the chip, a model can determine a movement vector for the stage and a movement for the imaging plane, (iii) depending on whether such movement vector may take the chip closer to the optimum X, Y, and Z position relative to the image unit, an error term can be determined as a loss for the model, and (iv) the magnitude of the error can be either constant or be proportional to how far the current X, Y, and Z position is from an optimal X, Y, and Z position (e.g., may be predetermined). Such trained model can be used to determine, for example, the movement vector and/or movement of the movement for the imaging plane, to enhance relative alignment between the chip and the image unit (e.g., one or more sensors).

The alignment can occur subsequent to capturing of the image(s). Alternatively or in addition to, the alignment can occur real-time while capturing images/videos of the positioning identifier(s) of the chip.

One or more flow channels of the flow cell of the present disclosure may have various shapes and sizes. For example, referring to FIGS. 6A and 6B, at least a portion of the flow channel (e.g., the focusing region 1132, the ordering region 1134, the rotation region 1136, the imaging region 1138, connecting region therebetween, etc.) may have a cross-section that is circular, triangular, square, rectangular, pentagonal, hexagonal, or any partial shape or combination of shapes thereof.

In some embodiments, the system of the present disclosure comprises straight channels with rectangular or square cross-sections. In some aspects, the system of the present disclosure comprises straight channels with round cross-sections. In some aspects, the system comprises straight channels with half-ellipsoid cross-sections. In some aspects, the system comprises spiral channels. In some aspects, the system comprises round channels with rectangular cross-sections. In some aspects, the system comprises round channels with rectangular channels with round cross-sections. In some aspects, the system comprises round channels with half-ellipsoid cross-sections. In some aspects, the system comprises channels that are expanding and contracting in width with rectangular cross-sections. In some aspects, the system comprises channels that are expanding and contracting in width with round cross-sections. In some aspects, the system comprises channels that are expanding and contracting in width with half-ellipsoid cross-sections.

### Focusing Regions

The flow channel can comprise one or more walls that are formed to focus one or more cells into a streamline. The flow channel can comprise a focusing region comprising the wall(s) to focus the cell(s) into the streamline. Focusing regions on a microfluidic device can take a disorderly stream of cells and utilize a variety of forces (for e.g. inertial lift forces (wall effect and shear gradient forces) or hydrodynamic forces) to focus the cells within the flow into a streamline of cells. In some embodiments, the cells are focused in a single streamline. In some examples, the cells are focused in multiple streamlines, for example at least 2, at least 3, at least 4, at least 5, at least 6, at least 7, at least 8, at least 9, or at least 10 streamlines.

The focusing region receives a flow of randomly arranged cells via an upstream section. The cells flow into a region of contracted and expanded sections in which the randomly arranged cells are focused into a single streamline of cells. The focusing can be driven by the action of inertial lift forces (wall effect and shear gradient forces) acting on cells.

In some embodiments, the focusing region is formed with curvilinear walls that form periodic patterns. In some embodiments, the patterns form a series of square expansions and contractions. In other embodiments, the patterns are sinusoidal. In further embodiments, the sinusoidal patterns are skewed to form an asymmetric pattern. The focusing region can be effective in focusing cells over a wide range of flow rates. In the illustrated embodiment, an asymmetrical sinusoidal-like structure is used as opposed to square expansions and contractions. This helps prevent the formation of secondary vortices and secondary flows behind the particle flow stream. In this way, the illustrated structure allows for faster and more accurate focusing of cells to a single lateral equilibrium position. Spiral and curved channels can also be used in an inertia regime; however, these can complicate the integration with other modules. Finally, straight channels where channel width is greater than channel height can also be used for focusing cells onto single lateral position. However, in this case, since there will be more than one equilibrium position in the z-plane, imaging can become problematic, as the imaging focal plane is preferably fixed. As can readily be appreciated, any of a variety of structures that provide a cross section that expands and contracts along the length of the microfluidic channel or are capable of focusing the cells can be utilized as appropriate to the requirements of specific applications.

The cell sorting system can be configured to focus the cell at a width and/or a height within the flow channel along an axis of the flow channel. The cell can be focused to a center or off the center of the cross-section of the flow channel. The cell can be focused to a side (e.g., a wall) of the cross-section of the flow channel. A focused position of the cell within the cross-section of the channel may be uniform or non-uniform as the cell is transported through the channel.

While specific implementations of focusing regions within microfluidic channels are described above, any of a variety of channel configurations that focus cells into a single streamline can be utilized as appropriate to the requirements of a specific application in accordance with various embodiments of the disclosure.

### Ordering Regions

Microfluidic channels can be designed to impose ordering upon a single streamline of cells formed by a focusing region in accordance with several embodiments of the disclosure. Microfluidic channels in accordance with some embodiments of the disclosure include an ordering region having pinching regions and curved channels. The ordering region orders the cells and distances single cells from each other to facilitate imaging. In some embodiments, ordering is achieved by forming the microfluidic channel to apply inertial lift forces and Dean drag forces on the cells.

Different geometries, orders, and/or combinations can be used. In some embodiments, pinching regions can be placed downstream from the focusing channels without the use of curved channels. Adding the curved channels helps with more rapid and controlled ordering, as well as increasing the likelihood that particles follow a single lateral position as they migrate downstream. As can readily be appreciated, the specific configuration of an ordering region is largely determined based upon the requirements of a given application.

### Cell Rotating Regions and Imaging Regions

Architecture of the microfluidic channels of the flow cell of the present disclosure may be controlled (e.g., modified, optimized, etc.) to modulate cell flow along the microfluidic channels. Examples of the cell flow may include (i) cell focusing (e.g., into a single streamline) and (ii) rotation of the one or more cells as the cell(s) are migrating (e.g., within the single streamline) down the length of the microfluidic channels. In some embodiments, microfluidic channels can be configured to impart rotation on ordered cells in accordance with a number of embodiments of the disclosure. One or more cell rotation regions (e.g., the cell rotation region 1136) of microfluidic channels in accordance with some embodiments of the disclosure use co-flow of a particle-free buffer to induce cell rotation by using the co-flow to apply differential velocity gradients across the cells. In some cases, a cell rotation region may introduce co-flow of at least 1, 2, 3, 4, 5, or more buffers (e.g., particle-free, or containing one or more particles, such as polymeric or magnetic particles) to impart rotation on one or more cells within the channel. In some cases, a cell rotation region may introduce co-flow of at most 5, 4, 3, 2, or 1 buffer to impart the rotation of one or more cells within the channel. In some examples, the plurality of buffers may be co-flown at a same position along the length of the cell rotation region, or sequentially at different positions along the length of the cell rotation region. In some examples, the plurality of buffers may be the same or different. In several embodiments, the cell rotation region of the microfluidic channel is fabricated using a two-layer fabrication process so that the axis of rotation is perpendicular to the axis of cell downstream migration and parallel to cell lateral migration.

Cells may be imaged in at least a portion of the cell rotating region, while the cells are tumbling and/or rotating as they migrate downstream. Alternatively or in addition to, the cells may be imaged in an imaging region that is adjacent to or downstream of the cell rotating region. In some examples, the cells may be flowing in a single streamline within a flow channel, and the cells may be imaged as the cells are rotating within the single streamline. A rotational speed of the cells may be constant or varied along the length of the imaging region. This may allow for the imaging of a cell at different angles (e.g., from a plurality of images of the cell taken from a plurality of angles due to rotation of the cell), which may provide more accurate information concerning cellular features than can be captured in a single image or a sequence of images of a cell that is not rotating to any significant extent. This also allow a 3D reconstruction of the cell using available software since the angles of rotation across the images are known. Alternatively, every single image of the sequence of image many be analyzed individually to analyze (e.g., classify) the cell from each image. In some cases, results of the individual analysis of the sequence of images may be aggregated to determine a final decision (e.g., classification of the cell).

In some embodiments, a cell rotation region of a microfluidic channel incorporates an injected co-flow prior to an imaging region in accordance with an embodiment of the disclosure. Co-flow may be introduced in the z plane (perpendicular to the imaging plane) to spin the cells. Since the imaging is done in the x-y plane, rotation of cells around an axis parallel to the y-axis provides additional information by rotating portions of the cell that may have been occluded in previous images into view in each subsequent image. Due to a change in channel dimensions, at point xo, a velocity gradient is applied across the cells, which can cause the cells to spin. The angular velocity of the cells depends on channel and cell dimensions and the ratio between Q1 (main channel flow rate) and Q2 (co-flow flow rate) and can be configured as appropriate to the requirements of a given application. In some embodiments, a cell rotation region incorporates an increase in one dimension of the microfluidic channel to initiate a change in the velocity gradient across a cell to impart rotation onto the cell. In some aspects, a cell rotation region of a microfluidic channel incorporates an increase in the z-axis dimension of the cross section of the microfluidic channel prior to an imaging region in accordance with an embodiment of the disclosure. The change in channel height can initiate a change in velocity gradient across the cell in the z axis of the microfluidic channel, which can cause the cells to rotate as with using co-flow.

### Flowing Cells

In some embodiments, the system and methods of the present disclosure focuses the cells in microfluidic channels. The term focusing as used herein broadly means controlling the trajectory of cell/cells movement and comprises controlling the position and/or speed at which the cells travel within the microfluidic channels. In some embodiments controlling the lateral position and/or the speed at which the particles travel inside the microfluidic channels, allows to accurately predict the time of arrival of the cell at a bifurcation. The cells may then be accurately sorted. The parameters critical to the focusing of cells within the microfluidic channels include, but are not limited to channel geometry, particle size, overall system throughput, sample concentration, imaging throughput, size of field of view, and method of sorting.

In some embodiments the focusing is achieved using inertial forces. In some embodiments, the system and methods of the present disclosure focus cells to a certain height from the bottom of the channel using inertial focusing. In these embodiments, the distance of the cells from the objective is equal and images of all the cells will be clear. As such, cellular details, such as nuclear shape, structure, and size appear clearly in the outputted images with minimal blur. In some aspects, the system disclosed herein has an imaging focusing plane that is adjustable. In some aspects, the focusing plane is adjusted by moving the objective or the stage. In some aspects, the best focusing plane is found by recording videos at different planes and the plane wherein the imaged cells have the highest Fourier magnitude, thus, the highest level of detail and highest resolution, is the best plane.

In some embodiments, the system and methods of the present disclosure utilize a hydrodynamic-based z focusing system to obtain a consistent z height for the cells of interests that are to be imaged. In some aspects, the design comprises hydrodynamic focusing using multiple inlets for main flow and side flow. In some aspects, the hydrodynamic-based z focusing system is a triple-punch design. In some aspects, the design comprises hydrodynamic focusing with three inlets, wherein the two side flows pinch cells at the center. For certain channel designs, dual z focus points may be created, wherein a double-punch design similar to the triple-punch design may be used to send objects to one of the two focus points to get consistent focused images. In some aspects, the design comprises hydrodynamic focusing with 2 inlets, wherein only one side flow channel is used and cells are focused near channel wall. In some aspects, the hydrodynamic focusing comprises side flows that do not contain any cells and a middle inlet that contains cells. The ratio of the flow rate on the side channel to the flow rate on the main channel determines the width of cell focusing region. In some aspects, the design is a combination of the above. In all aspects, the design is integrable with the bifurcation and sorting mechanisms disclosed herein. In some aspects, the hydrodynamic-based z focusing system is used in conjunction with inertia-based z focusing.

In some embodiments, the terms “particles”, “objects”, and “cells” are used interchangeably. In some aspects, the cell is a live cell. In some aspects, the cell is a fixed cell (e.g., in methanol or paraformaldehyde). In some cases, one or more cells may be coupled (e.g., attached covalently or non-covalently) to a substrate (e.g., a polymeric bead or a magnetic bead) while flowing through the flow cell. In some cases, the cell(s) may not be coupled to any substrate while flowing through the flow cell.

### Imaging and Classification

A variety of techniques can be utilized to classify images of cells captured by classification and/or sorting systems in accordance with various embodiments of the disclosure. In some embodiments, the image captures are saved for future analysis/classification either manually or by image analysis software. Any suitable image analysis software can be used for image analysis. In some embodiments, image analysis is performed using OpenCV. In some embodiments, analysis and classification is performed in real time.

In some embodiments, the system and methods of the present disclosure comprise collecting a plurality of images of objects in the flow. In some aspects, the plurality of images comprises at least 20 images of cells. In some aspects, the plurality of images comprises at least 19, 18, 17, 16, 15, 14, 13, 12, 11, 10, 9, 8, 7, 6, 5, 4, 3, or 2 images of cells. In some embodiments, the plurality of images comprises images from multiple cell angles. In some aspects, the plurality of images, comprising images from multiple cell angles, help derive extra features from the particle which would typically be hidden if the particle is imaged from a single point-of-view. In some aspects, without wishing to be bound by theory, the plurality of images, comprising images from multiple cell angles, help derive extra features from the particle which would typically be hidden if a plurality of images are combined into a multi-dimensional reconstruction (e.g., a two-dimensional hologram or a three-dimensional reconstruction).

In some embodiments, the systems and methods of present disclosure allow for a tracking ability, wherein the system and methods track a particle (e.g., cell) under the camera and maintain the knowledge of which frames belong to the same particle. In some embodiments, the particle is tracked until it has been classified and/or sorted. In some cases, the particle may be tracked by one or more morphological (e.g., shape, size, area, volume, texture, thickness, roundness, etc.) and/or optical (e.g., light emission, transmission, reflectance, absorbance, fluorescence, luminescence, etc.) characteristics of the particle. In some examples, each particle may be assigned a score (e.g., a characteristic score) based on the one or more morphological and/or optical characteristics, thereby to track and confirm the particle as the particle travels through the microfluidic channel.

In some embodiments, the systems and methods of the disclosure comprise imaging a single particle in a particular field of view of the camera. In some aspects, the system and methods of the present disclosure image multiple particles in the same field of view of camera. Imaging multiple particles in the same field of view of the camera can provide additional advantages, for example it will increase the throughput of the system by batching the data collection and transmission of multiple particles. In some instances, at least about 2, 3, 4, 5, 6, 7, 8, 9, 10, 20, 30, 40, 50, 60, 70, 80, 90, 100, or more particles are imaged in the same field of view of the camera. In some instances, 100 to 200 particles are imaged in the same field of view of the camera. In some instances, at most about 100, 90, 80, 70, 60, 50, 40, 30, 20, 10, 9, 8, 7, 6, 5, 4, 3, or 2 particles are imaged in the same field of view of the camera. In some cases, the number of the particles (e.g., cells) that are imaged in the same field of view may not be changed throughout the operation of the flow cell. Alternatively, the number of the particles (e.g., cells) that are imaged in the same field of view may be changed in real-time throughout the operation of the flow cell, e.g., to increase speed of the classification and/or sorting process without negatively affecting quality or accuracy of the classification and/or soring process.

The imaging region maybe downstream of the focusing region and the ordering region. Thus, the imaging region may not be part of the focusing region and the ordering region. In an example, the focusing region may not comprise or be operatively coupled to any imaging device that is configured to capture one or more images to be used for particle analysis (e.g., cell classification).

Sorting

In some embodiments, the systems and the methods of the present disclosure actively sorts a stream of particles. The term sort or sorting as used herein refers to physically separating particles, for e.g. cells, with one or more desired characteristics. The desired characteristic(s) can comprise a feature of the cell(s) analyzed and/or obtained from the image(s) of the cell. Examples of the feature of the cell(s) can comprise a size, shape, volume, electromagnetic radiation absorbance and/or transmittance (e.g., fluorescence intensity, luminescence intensity, etc.), or viability (e.g., when live cells are used).

The flow channel can branch into a plurality of channels, and the cell sorting system can be configured to sort the cell by directing the cell to a selected channel of the plurality of channels based on the analyzed image of the cell. The analyzed image may be indicative of one or more features of the cell, wherein the feature(s) are used as parameters of cell sorting. In some cases, one or more channels of the plurality of channels can have a plurality of sub-channels, and the plurality of sub-channels can be used to further sort the cells that have been sorted once.

Cell sorting may comprise isolating one or more target cells from a population of cells. The target cell(s) may be isolated into a separate reservoir that keeps the target cell(s) separate from the other cells of the population. Cell sorting accuracy may be defined as a proportion (e.g., a percentage) of the target cells in the population of cells that have been identified and sorted into the separate reservoir. In some cases, the cell sorting accuracy of the flow cell provided herein may be at least 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.9% or 100%). In some cases, the cell sorting accuracy of the flow cell provided herein may be at most 100%, 99%, 98%, 7%, 96%, 95%, 94%, 93%, 92%, 91%, 90%, 89%, 88%, 87%, 86%, 85%, 84%, 83%, 82%, 81%, 80%, or less.

In some cases, cell sorting may be performed at a rate of at least 1 cell/second, 5 cells/second, 10 cells/second, 50 cells/second, 100 cells/second, 500 cells/second, 1,000 cells/second, 5,000 cells/second, 10,000 cells/second, 50,000 cells/second, or more. In some cases, cell sorting may be performed at a rate of at most 50,000 cells/second, 10,000 cells/second, 5,000 cells/second, 1,000 cells/second, 500 cells/second, 100 cells/second, 50 cells/second, 10 cells/second, 5 cells/second, 1 cell/second, or less.

In some aspects, the systems and methods disclosed herein use an active sorting mechanism. In various embodiments, the active sorting is independent from analysis and decision making platforms and methods. In various embodiments the sorting is performed by a sorter, which receives a signal from the decision making unit (e.g. a classifier), or any other external unit, and then sorts cells as they arrive at the bifurcation. The term bifurcation as used herein refers to the termination of the flow channel into two or more channels, such that cells with the one or more desired characteristics are sorted or directed towards one of the two or more channels and cell without the one or more desired characteristics are directed towards the remaining channels. In some embodiments, the flow channel terminates into at least 2, 3, 4, 5, 6, 7, 8, 9, 10, or more channels. In some embodiments, the flow channel terminates into at most 10, 9, 8, 7, 6, 5, 4, 3, or 2 channels. In some embodiments, the flow channel terminates in two channels and cells with one or more desired characteristics are directed towards one of the two channels (the positive channel), while cells without the one or more desired characteristics are directed towards the other channel (the negative channel). In some embodiments, the flow channel terminates in three channels and cells with a first desired characteristic are directed to one of the three channels, cells with a second desired characteristic are directed to another of the three channels, and cells without the first desired characteristic and the second desired characteristic are directed to the remaining of the three channels.

In some embodiments, the sorting is performed by a sorter. The sorter may function by predicting the exact time at which the particle will arrive at the bifurcation. To predict the time of particle arrival, the sorter can use any applicable method. In some examples, the sorter predicts the time of arrival of the particle by using (i) velocity of particles (e.g., downstream velocity of a particle along the length of the microfluidic channel) that are upstream of the bifurcation and (ii) the distance between velocity measurement/calculation location and the bifurcation. In some examples, the sorter predicts the time of arrival of the particles by using a constant delay time as an input.

In some cases, prior to the cell's arrival at the bifurcation, the sorter may measure the velocity of a particle (e.g., a cell) at least 1, 2, 3, 4, 5, or more times. In some cases, prior to the cell's arrival at the bifurcation, the sorter may measure the velocity of the particle at most 5, 4, 3, 2, or 1 time. In some cases, the sorter may use at least 1, 2, 3, 4, 5, or more sensors. In some cases, the sorter may use at most 5, 4, 3, 2, or 1 sensor. Example of the sensor(s) may be an imaging device (e.g., a camera such as a high-speed camera), one- or multi-point light (e.g., laser) detector, etc. Referring to FIGS. 6A and 6B, the sorter may use any one of the imaging devices (e.g., the high-speed camera system 114) disposed at or adjacent to the imaging region 1138. In some examples, the same imaging device(s) may be used to capture one or more images of a cell as the cell is rotating and migrating within the channel, and the one or more images may be analyzed to (i) classify the cell and (ii) measure a rotational and/or lateral velocity of the cell within the channel and predict the cell's arrival time at the bifurcation. In some examples, the sorter may use one or more sensors that are different than the imaging devices of the imaging region 1138. The sorter may measure the velocity of the particle (i) upstream of the imaging region 1138, (ii) at the imaging region 1138, and/or (iii) downstream of the imaging region 1138.

The sorter may comprise or be operatively coupled to a processor, such as a computer processor. Such processor may be the processor 1116 that is operatively coupled to the imaging device 114 or a different processor. The processor may be configured to calculate the velocity of a particle (rotational and/or downstream velocity of the particle) an predict the time of arrival of the particle at the bifurcation. The processor may be operatively coupled to one or more valves of the bifurcation. The processor may be configured to direct the valve(s) to open and close any channel in fluid communication with the bifurcation. The processor may be configured to predict and measure when operation of the valve(s) (e.g., opening or closing) is completed.

In some examples, the sorter may comprise a self-included unit (e.g., comprising the sensors, such as the imaging device(s)) which is capable of (i) predicting the time of arrival of the articles and/or (ii) detecting the particle as it arrives at the bifurcation. In order to sort the particles, the order at which the particles arrive at the bifurcation, as detected by the self-included unit, may be matched to the order of the received signal from the decision making unit (e.g. a classifier). In some aspects, controlled particles are used to align and update the order as necessary. In some examples, the decision making unit may classify a first cell, a second cell, and a third cell, respectively, and the sorter may confirm that the first cell, the second cell, and the third cell are sorted, respectively in the same order. If the order is confirmed, the classification and sorting mechanisms (or deep learning algorithms) may remain the same. If the order is different between the classifying and the sorting, then the classification and/or sorting mechanisms (or deep learning algorithms) may be updated or optimized, either manually or automatically. In some aspects, the controlled particles may be cells (e.g., live or dead cells).

In some aspects, the controlled particles may be special calibration beads (e.g., plastic beads, metallic beads, magnetic beads, etc.). In some embodiments the calibration beads used are polystyrene beads with size ranging between about 1 μM to about 50 μM. In some embodiments the calibration beads used are polystyrene beads with size of least about 1 μM. In some embodiments the calibration beads used are polystyrene beads with size of at most about 50 μM. In some embodiments the calibration beads used are polystyrene beads with size ranging between about 1 μM to about 3 about 1 μM to about 5 about 1 μM to about 6 about 1 μM to about 10 about 1 μM to about 15 about 1 μM to about 20 about 1 μM to about 25 about 1 μM to about 30 about 1 μM to about 35 about 1 μM to about 40 about 1 μM to about 50 about 3 μM to about 5 about 3 μM to about 6 about 3 μM to about 10 about 3 μM to about 15 about 3 μM to about 20 about 3 μM to about 25 about 3 μM to about 30 about 3 μM to about 35 about 3 μM to about 40 about 3 μM to about 50 about 5 μM to about 6 about 5 μM to about 10 about 5 μM to about 15 about 5 μM to about 20 about 5 μM to about 25 about 5 μM to about 30 about 5 μM to about 35 about 5 μM to about 40 about 5 μM to about 50 about 6 μM to about 10 about 6 μM to about 15 about 6 μM to about 20 about 6 μM to about 25 about 6 μM to about 30 about 6 μM to about 35 μM, about 6 μM to about 40 μM, about 6 μM to about 50 μM, about 10 μM to about 15 μM, about 10 μM to about 20 μM, about 10 μM to about 25 μM, about 10 μM to about 30 μM, about 10 μM to about 35 μM, about 10 μM to about 40 μM, about 10 μM to about 50 μM, about 15 μM to about 20 μM, about 15 μM to about 25 μM, about 15 μM to about 30 μM, about 15 μM to about 35 μM, about 15 μM to about 40 μM, about 15 μM to about 50 μM, about 20 μM to about 25 μM, about 20 μM to about 30 μM, about 20 μM to about 35 μM, about 20 μM to about 40 μM, about 20 μM to about 50 μM, about 25 μM to about 30 μM, about 25 μM to about 35 μM, about 25 μM to about 40 μM, about 25 μM to about 50 μM, about 30 μM to about 35 μM, about 30 μM to about 40 μM, about 30 μM to about 50 μM, about 35 μM to about 40 μM, about 35 μM to about 50 μM, or about 40 μM to about 50 μM. In some embodiments the calibration beads used are polystyrene beads with size of about 1 μM, about 3 μM, about 5 μM, about 6 μM, about 10 μM, about 15 μM, about 20 μM, about 25 μM, about 30 μM, about 35 μM, about 40 μM, or about 50 μM.

In some embodiments, the sorter (or an additional sensor disposed at or adjacent to the bifurcation) may be configured to validate arrival of the particles (e.g., the cells) at the bifurcation. In some examples, the sorter may be configured to measure an actual arrival time of the particles (e.g., the cells) at the bifurcation. The sorter may analyze (e.g., compare) the predicted arrival time, the actual arrival time, the velocity of the particles downstream of the channel prior to any adjustment of the velocity, and/or a velocity of the particles downstream of the channel subsequent to such adjustment of the velocity. Based on the analyzing, the sorter may modify any operation (e.g., cell focusing, cell rotation, controlling cell velocity, cell classification algorithms, valve actuation processes, etc.) of the flow cell. The validation by the sorter may be used for closed-loop and real-time update of any operation of the flow cell.

In some cases, to predict the time of arrival of one or more cells for sorting, the systems, methods, and platforms disclosed herein can dynamically adjust a delay time (e.g., a constant delay time) based on imaging of the cell(s) or based on tracking of the cell(s) with light (e.g., laser). By detecting changes (e.g., flow rates, velocity of aggregate of multiple cells, the lateral location of cells in the channel, etc.) the delay time (e.g., time at which the cells arrive at the bifurcation) can be predicted and adjusted in real-time (e.g., every few milliseconds). A feedback loop can be designed that can constantly read such changes and adjust the delay time accordingly. Alternatively or in addition to, the delay time can be adjusted for each cell/particle. The delay time can be calculated separately for each individual cell, based on, e.g., its velocity, lateral position in the channel, and/or time of arrival at specific locations along the channel (e.g., using tracking based on lasers or other methods). The calculated delay time can then be applied to the individual cell/particle (e.g., if the cell is a positive cell or a target cell, the sorting can be performed according to its specific delay time or a predetermined delay time).

In some embodiments, the sorters used in the systems and methods disclosed herein are self-learning cell sorting systems or intelligent cell sorting systems, as disclosed herein. These sorting systems can continuously learn based on the outcome of sorting. For example, a sample of cells is sorted, the sorted cells are analyzed, and the results of this analysis are fed back to the classifier. In some examples, the cells that are sorted as “positive” (i.e., target cells or cells of interest) may be analyzed and validated. In some examples, the cells that are sorted as “negative” (i.e., non-target cells or cells not of interest) may be analyzed and validated. In some examples, both positive and negative cells may be validated. Such validation of sorted cells (e.g., based on secondary imaging and classification) may be used for closed-loop and real-time update of the primary cell classification algorithms.

In some cases, a flush mechanism can be used during sorting. The flush mechanism can ensure that the cell which has been determined to be sorted to a specific bucket or well will end up there (e.g., not be stuck in various parts of the channel or outlet). The flush mechanism can ensure that the channel and outlets stay clean and debris-free for maximum durability. The flush mechanism can inject additional solutions/reagents (e.g., cell lysis buffers, barcoded reagents, etc.) to the well or droplet that the cell is being sorted into. The flush mechanism can be supplied by a separate set of channels and/or valves which are responsible to flow a fluid at a predefined cadence in the direction of sorting.

### Sorting Techniques

In some embodiments, the methods and systems disclosed herein can use any sorting technique to sort particles. At least a portion of the collection reservoir may or may not be pre-filled with a fluid, e.g., a buffer. In some embodiments, the sorting technique comprises closing a channel on one side of the bifurcation to collect the desired cell on the other side. In some aspects, the closing of the channels can be carried out by employing any known technique. In some aspects, the closing is carried out by application of a pressure. In some instances, the pressure is pneumatic actuation. In some aspects, the pressure can be positive pressure or negative pressure. In some embodiments, positive pressure is used. In some examples, one side of the bifurcation is closed by applying pressure and deflecting the soft membrane between top and bottom layers. Other aspects of systems and methods of particle (e.g., cell) imaging, analysis, and sorting are further described in International Application No. PCT/US2017/033676 and International Application No. PCT/US2019/046557, each of which is incorporated herein by reference.

### Sample and Data Collection

In various embodiments, the systems and methods of the present disclosure comprise one or more reservoirs designed to collect the particles after the particles have been sorted. In some embodiments, the number of cells to be sorted is about 1 cell to about 1,000,000 cells. In some embodiments, the number of cells to be sorted is at least about 1 cell. In some embodiments, the number of cells to be sorted is at most about 1,000,000 cells. In some embodiments, the number of cells to be sorted is about 1 cell to about 100 cells, about 1 cell to about 500 cells, about 1 cell to about 1,000 cells, about 1 cell to about 5,000 cells, about 1 cell to about 10,000 cells, about 1 cell to about 50,000 cells, about 1 cell to about 100,000 cells, about 1 cell to about 500,000 cells, about 1 cell to about 1,000,000 cells, about 100 cells to about 500 cells, about 100 cells to about 1,000 cells, about 100 cells to about 5,000 cells, about 100 cells to about 10,000 cells, about 100 cells to about 50,000 cells, about 100 cells to about 100,000 cells, about 100 cells to about 500,000 cells, about 100 cells to about 1,000,000 cells, about 500 cells to about 1,000 cells, about 500 cells to about 5,000 cells, about 500 cells to about 10,000 cells, about 500 cells to about 50,000 cells, about 500 cells to about 100,000 cells, about 500 cells to about 500,000 cells, about 500 cells to about 1,000,000 cells, about 1,000 cells to about 5,000 cells, about 1,000 cells to about 10,000 cells, about 1,000 cells to about 50,000 cells, about 1,000 cells to about 100,000 cells, about 1,000 cells to about 500,000 cells, about 1,000 cells to about 1,000,000 cells, about 5,000 cells to about 10,000 cells, about 5,000 cells to about 50,000 cells, about 5,000 cells to about 100,000 cells, about 5,000 cells to about 500,000 cells, about 5,000 cells to about 1,000,000 cells, about 10,000 cells to about 50,000 cells, about 10,000 cells to about 100,000 cells, about 10,000 cells to about 500,000 cells, about 10,000 cells to about 1,000,000 cells, about 50,000 cells to about 100,000 cells, about 50,000 cells to about 500,000 cells, about 50,000 cells to about 1,000,000 cells, about 100,000 cells to about 500,000 cells, about 100,000 cells to about 1,000,000 cells, or about 500,000 cells to about 1,000,000 cells. In some embodiments, the number of cells to be sorted is about 1 cell, about 100 cells, about 500 cells, about 1,000 cells, about 5,000 cells, about 10,000 cells, about 50,000 cells, about 100,000 cells, about 500,000 cells, or about 1,000,000 cells.

In some embodiments, the number of cells to be sorted is 100 to 500 cells, 200 to 500 cells, 300 to 500 cells, 350 to 500 cells, 400 to 500 cells, or 450 to 500 cells. In some embodiments, the reservoirs may be milliliter scale reservoirs. In some examples, the one or more reservoirs are pre-filled with a buffer and the sorted cells are stored in the buffer. Using the buffer helps to increase the volume of the cells, which can then be easily handled, for example a pipetted. In some examples, the buffer is a phosphate buffer, for example phosphate-buffered saline (PBS).

In some embodiments, the system and methods of the present disclosure comprise a cell sorting technique wherein pockets of buffer solution containing no negative objects are sent to the positive output channel in order to push rare objects out of the collection reservoir. In some aspects, additional buffer solution is sent to the positive output channel to flush out all positive objects at the end of a run, once the channel is flushed clean (e.g., using the flush mechanism as disclosed herein).

In some embodiments, the system and methods of the present disclosure comprise a cell retrieving technique, wherein sorted cells can be retrieved for downstream analysis (e.g., molecular analysis). Non-limiting examples of the cell retrieving technique can include: retrieval by centrifugation; direct retrieval by pipetting; direct lysis of cells in well; sorting in a detachable tube; feeding into a single cell dispenser to be deposited into 96 or 384 well plates; etc.

### Real-Time Integration

In some embodiments, the system and methods of the present disclosure comprise a combination of techniques, wherein a graphics processing unit (GPU) and a digital signal processor (DSP) are used to run artificial intelligence (AI) algorithms and apply classification results in real-time to the system. In some aspects, the system and methods of the present disclosure comprise a hybrid method for real-time cell sorting.

In some embodiments, the system and methods of the present disclosure comprise a feedback loop (e.g., an automatic feedback loop). For example, the system and methods can be configured to (i) monitor the vital signals and (ii) finetune one or more parameters of the system and methods based on the signals being read. At the beginning or throughout the run (e.g., the use of the microfluidic channel for cell imaging, classification, and/or sorting), a processor (e.g., a ML/AI processor as disclosed herein) can specify target values for one or more selected parameters (e.g., flow rate, cell rate, etc.). Alternatively or in addition to, other signals that reflect (e.g., automatically reflect) the quality of the run (e.g., the number of cells that are out of focus within the last 100 imaged cells) can be utilized in the feedback loop. The feedback loop can receive (e.g., in real-time) values of the parameters/signals disclosed herein and, based on the predetermined target values and/or one or more general mandates (e.g., the fewer the out-of-focus cells, the better), the feedback loop can facilitate adjustments (e.g., adjustments to pressure systems, illumination, stage, etc.). In some cases, the feedback loop can be designed to monitor and/or handle degenerate scenarios, in which the microfluidic system is not responsive or mal-functioning (e.g., outputting a value read that is out of range of acceptable reads).

In some embodiments, the system and methods of the present disclosure can adjust a cell classification threshold based on expected true positive rate for a sample type. The expected true positive rate can come from statistics gathered in one or more previous runs from the same or other patients with similar conditions. Such approach can help neutralize run-to-run variations (e.g., illumination, chip fabrication variation, etc.) that would impact imaging and hence any inference therefrom.

### Validation

In some embodiments, the systems disclosed herein further comprise a validation unit that detects the presence of a particle without getting detailed information, such as imaging. In some instances, the validation unit may be used for one or more purposes. In some examples, the validation unit detects a particle approaching the bifurcation and enables precise sorting. In some examples, the validation unit detects a particle after the particle has been sorted to one of subchannels in fluid communication with the bifurcation. In some examples, the validation unit provides timing information with a plurality of laser spots, e.g., two laser spots. In some instances, the validation unit provides timing information by referencing the imaging time. In some instances, the validation unit provides precise time delay information and/or flow speed of particles.

### Samples

In some embodiments, the particles (for e.g. cells) analyzed by the systems and methods disclosed herein are comprised in a sample. The sample may be a biological sample obtained from a subject. In some embodiments, the biological sample comprises a biopsy sample from a subject. In some embodiments, the biological sample comprises a tissue sample from a subject. In some embodiments, the biological sample comprises liquid biopsy from a subject. In some embodiments, the biological sample can be a solid biological sample, e.g., a tumor sample. In some embodiments, a sample from a subject can comprise at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% tumor cells from a tumor.

In some embodiments, the sample can be a liquid biological sample. In some embodiments, the liquid biological sample can be a blood sample (e.g., whole blood, plasma, or serum). A whole blood sample can be subjected to separation of cellular components (e.g., plasma, serum) and cellular components by use of a Ficoll reagent. In some embodiments, the liquid biological sample can be a urine sample. In some embodiments, the liquid biological sample can be a perilymph sample. In some embodiments, the liquid biological sample can be a fecal sample. In some embodiments, the liquid biological sample can be saliva. In some embodiments, the liquid biological sample can be semen. In some embodiments, the liquid biological sample can be amniotic fluid. In some embodiments, the liquid biological sample can be cerebrospinal fluid. In some embodiments, the liquid biological sample can be bile. In some embodiments, the liquid biological sample can be sweat. In some embodiments, the liquid biological sample can be tears. In some embodiments, the liquid biological sample can be sputum. In some embodiments, the liquid biological sample can be synovial fluid. In some embodiments, the liquid biological sample can be vomit.

In some embodiments, samples can be collected over a period of time and the samples may be compared to each other or with a standard sample using the systems and methods disclosed herein. In some embodiments the standard sample is a comparable sample obtained from a different subject, for example a different subject that is known to be healthy or a different subject that is known to be unhealthy. Samples can be collected over regular time intervals, or can be collected intermittently over irregular time intervals.

In some embodiments, the subject may be an animal (e.g., human, rat, pig, horse, cow, dog, mouse). In some instances, the subject is a human and the sample is a human sample. The sample may be a fetal human sample. The sample may be a placental sample (e.g., comprising placental cells). The sample may be from a multicellular tissue (e.g., an organ (e.g., brain, liver, lung, kidney, prostate, ovary, spleen, lymph node, thyroid, pancreas, heart, skeletal muscle, intestine, larynx, esophagus, and stomach), a blastocyst). The sample may be a cell from a cell culture. In some sample the subject is a pregnant human, or a human suspected to be pregnant.

The sample may comprise a plurality of cells. The sample may comprise a plurality of the same type of cell. The sample may comprise a plurality of different types of cells. The sample may comprise a plurality of cells at the same point in the cell cycle and/or differentiation pathway. The sample may comprise a plurality of cells at different points in the cell cycle and/or differentiation pathway.

The plurality of samples may comprise one or more malignant cell. The one or more malignant cells may be derived from a tumor, sarcoma, or leukemia.

The plurality of samples may comprise at least one bodily fluid. The bodily fluid may comprise blood, urine, lymphatic fluid, saliva. The plurality of samples may comprise at least one blood sample.

The plurality of samples may comprise at least one cell from one or more biological tissues. The one or more biological tissues may be a bone, heart, thymus, artery, blood vessel, lung, muscle, stomach, intestine, liver, pancreas, spleen, kidney, gall bladder, thyroid gland, adrenal gland, mammary gland, ovary, prostate gland, testicle, skin, adipose, eye or brain.

The biological tissue may comprise an infected tissue, diseased tissue, malignant tissue, calcified tissue or healthy tissue.

### Non-Invasive Prenatal Testing (NIPT)

Conventional prenatal screening methods for detecting fetal abnormalities and for sex determination use fetal samples acquired through invasive techniques, such as amniocentesis and chorionic villus sampling (CVS). Ultrasound imaging is also used to detect structural malformations such as those involving the neural tube, heart, kidney, limbs and the like. Chromosomal aberrations such as the presence of extra chromosomes, such as Trisomy 21 (Down syndrome), Klinefelter's syndrome, Trisomy 13 (Patau syndrome), Trisomy 18 (Edwards syndrome), or the absence of chromosomes, such as Turner's syndrome, or various translocations and deletions can be currently detected using CVS and/or amniocentesis. Both techniques require careful handling and present a degree of risk to the mother and to the pregnancy.

Prenatal diagnosis is offered to women over the age of 35 and/or women who are known to carry genetic diseases, as balanced translocations or microdeletions.

Chorionic villus sampling (CVS) is performed between the 9^{th} and the 14^{th} week of gestation. CVS involves the insertion of a catheter through the cervix or the insertion of a needle into the abdomen of the subject/patient. The needle or catheter is used to remove a small sample of the placenta, known as the chorionic villus. The fetal karyotype is then determined within one to two weeks of the CVS procedure. Due to the invasive nature of the CVS procedure, there is a 2 to 4% procedure-related risk of miscarriage. CVS is also associated with an increased risk of fetal abnormalities, such as defective limb development, which are presumably due to hemorrhage or embolism from the aspirated placental tissues.

Amniocentesis is performed between the 16^{th} and the 20^{th} week of gestation. Amniocentesis involves the insertion of a thin needle through the abdomen into the uterus of the patient. This procedure carries a 0.5 to 1% procedure-related risk of miscarriage. Amniotic fluid is aspirated by the needle and fetal fibroblast cells are further cultured for 1 to 2 weeks, following which they are subjected to cytogenetic and/or fluorescence in situ hybridization (FISH) analyses.

Recent techniques have been developed to predict fetal abnormalities and predict possible complications in pregnancy. These techniques use material blood or serum samples and have focused on the use of three specific markers, including alpha-fetoprotein (AFP), human chorionic gonadotrophin (hCG), and estriol. These three markers are used to screen for Down's syndrome and neural tube defects. Maternal serum is currently being used for biochemical screening for chromosomal aneuploidies and neural tube defects.

The passage of nucleated cells between the mother and fetus is a well-studied phenomenon. Using the fetal cells that are present in maternal blood for non-invasive prenatal diagnosis prevents the risks that are usually associated with conventional invasive techniques. Fetal cells include fetal trophoblasts, leukocytes, and nucleated erythrocytes from the maternal blood during the first trimester of pregnancy. This the, the isolation of trophoblasts from the maternal blood is limited by their multinucleated morphology and the availability of antibodies, whereas the isolation of leukocytes is limited by the lack of unique cell markers which differentiate maternal from fetal leukocytes. Furthermore, since leukocytes may persist in the maternal blood for as long as 27 years, residual cells are likely to be present in the maternal blood from previous pregnancies.

In some embodiments, the system and methods disclosed herein are used for non-invasive prenatal testing (NIPT), wherein the methods are used to analyze maternal serum or plasma samples from a pregnant female. In some aspects, the system and methods are used for non-invasive prenatal diagnosis. In some aspects, the system and methods disclosed herein can be used to analyze maternal serum or plasma samples derived from maternal blood. In some aspects, as little as 10 μL of serum or plasma can be used. In some aspects, larger samples are used to increase accuracy, wherein the volume of the sample used is dependent upon the condition or characteristic being detected.

In some embodiments, the system and methods disclosed herein are used for non-invasive prenatal diagnosis including but not limited to sex determination, blood typing and other genotyping, detection of pre-eclampsia in the mother, determination of any maternal or fetal condition or characteristic related to either the fetal DNA itself or the quantity or quality of the fetal DNA in the maternal serum or plasma, and identification of major or minor fetal malformations or genetic diseases present in a fetus. In some aspects, a fetus is a human fetus.

In some embodiments, the system and methods disclosed herein are used to analyze serum or plasma from maternal blood samples, wherein the serum or plasma preparation is carried out by standard techniques and subjected to a nucleic acid extraction process. In some aspects, the serum or plasma is extracted using a proteinase K treatment followed by phenol/chloroform extraction.

In some embodiments, the system and methods disclosed herein are used to image cells from maternal serum or plasma acquired from a pregnant female subject. In some aspects, the subject is a human. In some aspects, the pregnant female human subject is over the age of 35. In some aspects, the pregnant female human subject is known to carry a genetic disease. In some aspects, the subject is a human. In some aspects, the pregnant female human subject is over the age of 35 and is known to carry a genetic disease.

In some embodiments, the system and methods disclosed herein are used to analyze fetal cells from maternal serum or plasma. In some aspects, the cells that are used for non-invasive prenatal testing using the system and methods disclosed herein are fetal cells such as fetal trophoblasts, leukocytes, and nucleated erythrocytes. In some aspects, fetal cells are from the maternal blood during the first trimester of pregnancy.

In some embodiments, the system and methods disclosed herein are used for non-invasive prenatal diagnosis using fetal cells comprising trophoblast cells. In some aspects, trophoblast cells using the present disclosure are retrieved from the cervical canal using aspiration. In some aspects, trophoblast cells using the present disclosure are retrieved from the cervical canal using cytobrush or cotton wool swabs. In some aspects, trophoblast cells using the present disclosure are retrieved from the cervical canal using endocervical lavage. In some aspects, trophoblast cells using the present disclosure are retrieved from the cervical canal using intrauterine lavage.

In some embodiments, the system and methods disclosed herein are used to analyze fetal cells from maternal serum or plasma, wherein the cell population is mixed and comprises fetal cells and maternal cells. In some aspects, the system and methods of the present disclosure are used to identify embryonic or fetal cells in a mixed cell population. In some embodiments, the system and methods of the present disclosure are used to identify embryonic or fetal cells in a mixed cell population, wherein nuclear size and shape are used to identify embryonic or fetal cells in a mixed population. In some embodiments, the systems and methods disclosed herein are used to sort fetal cells from a cell population.

In some embodiments, the system and methods disclosed herein are used to measure the count of fetal nucleated red blood cells (RBCs), wherein an increase in fetal nucleated RBC count (or proportion) indicates the presence of fetal aneuploidy. In some examples, a control sample (e.g., a known blood or plasma sample from a non-pregnant individual) may be used for comparison. In some cases, the system and methods disclosed herein are used to provide a likelihood (i.e., probability) of a presence of an abnormal condition in a fetus.

In some embodiments, the system and methods disclosed herein are used to identify, classify, and/or measure the count of trophoblasts. In some cases, trophoblasts collected from the mother during a blood draw, can determine fetal genetic abnormalities.

In some embodiments, the system and methods disclosed herein are used to image cells from maternal serum or plasma acquired from a pregnant female subject. In some aspects, the cells are not labelled. In some aspects, the cells are in a flow. In some aspects, the cells are imaged from different angles. In some aspects, the cells are live cells. In some aspects, the cells are housed in a flow channel within the system of the present disclosure, wherein the flow channel has walls formed to space the plurality of cells within a single streamline. In some aspects, the cells are housed in a flow channel within the system of the present disclosure, wherein the flow channel has walls formed to rotate the plurality of the cells within a single streamline.

In some embodiments, the system and methods disclosed herein are used to image cells from maternal serum or plasma acquired from a pregnant female subject. In some aspects, a plurality of images of the cells is collected using the system and methods of the present disclosure. In some aspects, the plurality of images is analyzed to determine if specific disease conditions are present in the subject, wherein the cells are in a flow during the imaging and wherein the plurality of images comprises images of the cells from a plurality of angles. In some aspects, subject is the fetus. In some aspects, subject is pregnant female subject.

In some embodiments, the system and methods disclosed herein can classify and sort maternal or fetal cells, and the sorted material or fetal cells can be further analyzed for molecular analysis (e.g., genomics, proteomics, transcriptomics, etc.). In some cases, a mixture of maternal and fetal cells can be analyzed (e.g., as sub-pools or single-cells) for paired molecular analysis as disclosed herein.

### Sperm Analysis

In some embodiments, the sample used in the methods and systems described herein is a semen sample, and the system and methods of the present disclosure are used to identify sperm quality and/or gender. In these embodiments, the methods described herein comprise imaging the semen sample from the subject according to the methods described herein and analyzing the sperms in the semen sample for one or more features. In some embodiments, the systems and methods described herein are used to obtain a sperm count. In some aspects, the systems and methods described herein are used to obtain information about sperm viability and/or health. In some aspects, the systems and methods described herein are used to obtain information about sperm gender. In some embodiments, the sorting systems and methods described herein are used for and automated enrichment of sperms with desired morphological features. In some embodiment, the enriched sperms obtained according to the methods and systems described herein are used for in-vitro fertilization. In some aspects, the features are associated with health, motility, and/or gender.

### Circulating Endometrial Cells

In some embodiments, the system and methods disclosed herein can be utilized to detect circulating endometrial cells, e.g., for non-invasive diagnosis of endometriosis as an alternative or additional approach to other surgical methods (e.g., visualization or biopsy under laparoscopy). Determination of a presence of one or more endometrial cells in circulation in a provided sample, their count, their isolation, and/or subsequent molecular analysis (e.g., for gene expression consistent with endometriosis) can help detection of endometriosis. Similar approaches can be utilized for detection/analysis of circulating endometrial cancer cells, e.g., for uterine/endometrial cancer detection.

### Circulating Endothelial Cells

In some embodiments, the system and methods disclosed herein can be utilized to detect circulating endothelial cells. The endothelium can be involved (e.g., directly involved) in diseases such as, e.g., peripheral vascular disease, stroke, heart disease, diabetes, insulin resistance, chronic kidney failure, tumor growth, metastasis, venous thrombosis, and severe viral infectious diseases. Thus, dysfunction of the vascular endothelium can be one of the hallmarks of human diseases (e.g., preeclampsia (a pregnancy specific disease), endocarditis, etc.). For example, detection of circulating endothelial cells can be utilized for detection of cardiovascular disease. Sorted endothelial cells can be further analyzed for molecular profiling, e.g., specific vascular endothelial cell RNA expression in the presence of various vascular disease states.

### Cancer Cells

Many cancers are diagnosed in later stages of the disease because of low sensitivity of existing diagnostic procedures and processes. More than 1.5 million people are diagnosed with cancer every year in the USA, of which 600,000 people die. Currently, the first cancer screening procedure involves the detection of a tumor. Many cancer tumors, such as breast cancer are detected by self- or clinical examination. However, these tumors are typically detected only after the tumor reach a volume of 1 mL or 1 cc, when it contains approximately 10⁹ cells. Routine screening by mammography is more sensitive and allows detection of a tumor before it becomes palpable, but only after they reach an inch in diameter. MRI, positron emission tomography (PET) and single-photon emission computed tomography (SPECT) can reveal even smaller tumors than can be detected by mammograms. However, these imaging methods present significant disadvantages. Contrast agents for magnetic resonance imaging (MM) are toxic and radionuclides delivered for SPECT or PET examination are sources of ionizing radiation. Because of its relatively poor resolution, ovarian cancer often requires several follow up scans with computed tomography (CT) or MRI, while undertaking all precautions to protect possible pregnancies, to reveal fine anatomy of developing tumors. Additionally, all of these diagnostic techniques require dedicated facilities, expensive equipment, well trained staff, and financial coverages.

Cancer is commonly diagnosed in patients by obtaining a sample of the suspect tissue and examining the tissue under a microscope for the presence of malignant cells. While this process is relatively straightforward when the anatomic location of the suspect tissue is known, it can become quite challenging when there is no readily identifiable tumor or pre-cancerous lesion. For example, to detect the presence of lung cancer from a sputum sample requires one or more relatively rare cancer cells to be present in the sample. Therefore, patients having lung cancer may not be diagnosed properly if the sample does not perceptively and accurately reflect the conditions of the lung.

Conventional light microscopy, which utilizes cells mounted on glass slides, can only approximate 2D and 3D measurements because of limitations in focal plane depth, sampling angles, and problems with cell preparations that typically cause cells to overlap in the plane of the image. Another drawback of light microscopy is the inherent limitation of viewing through an objective lens where only the area within the narrow focal plane provides accurate data for analysis.

Flow cytometry methods generally overcome the cell overlap problem by causing cells to flow one-by-one in a fluid stream. Unfortunately, flow cytometry systems do not generate images of cells of the same quality as traditional light microscopy, and, in any case, the images are not three-dimensional.

In some embodiments, the system and methods disclosed herein enable the acquisition of three-dimensional imaging data of individual cells, wherein each individual cell from a cell population is imaged from a plurality of angles. In some aspects, the present disclosure is used to diagnose cancer, wherein individual cancer cells are identified, tracked, and grouped together. In some aspects, the cells are live.

In some embodiments, the system and methods disclosed herein are used for cancer diagnosis in a subject, the method comprising imaging a cell in a biological sample from the subject to collect a plurality of images of the cell and analyzing the plurality of images to determine if cancerous cells are present in the subject, wherein the cancerous cell is in a flow during imaging and is spinning, and wherein the plurality of images comprise images from a different spinning angles.

In some embodiments, the system and methods disclosed herein are used for cancer cell detection, wherein the cancerous cells are from biological samples and are detected and tracked as they pass through the system of the present disclosure.

In some embodiments, the system and methods disclosed herein are used to identify cancer cells from biological samples acquired from mammalian subjects, wherein the cell population is analyzed by nuclear detail, nuclear contour, presence or absence of nucleoli, quality of cytoplasm, quantity of cytoplasm, nuclear aspect ratio, cytoplasmic aspect ratio, or nuclear to cytoplasmic ratio. In some aspects, the cancer cells that are identified indicate the presence of cancer in the mammalian sample, including but not limited to, lymphoma, myeloma, neuroblastoma, breast cancer, ovarian cancer, lung cancer, rhabdomyosarcoma, small-cell lung tumors, primary brain tumors, stomach cancer, colon cancer, pancreatic cancer, urinary bladder cancer, testicular cancer, lymphomas, thyroid cancer, neuroblastoma, esophageal cancer, genitourinary tract cancer, cervical cancer, endometrial cancer, adrenal cortical cancer, or prostate cancer. In some aspects, the the cancer is metastatic cancer. In some aspects, the the cancer is an early stage cancer.

In some embodiments, the system and methods disclosed herein are used to image a large number of cells from a subject and collect a plurality of images of the cell, and to then classify the cells based on an analysis of one or more of the plurality of images; wherein the plurality of images comprise images from a plurality of cell angles and wherein the cell is tracked until the cell has been classified. In some aspects, the tracked cells are classified as cancerous. In some aspects, the subject is a human.

In some embodiments, the cells used in the methods disclosed herein are live cells. In some aspects, the cells that are classified as cancerous cells are isolated and subsequently cultured for potential drug compound screening, testing of a biologically active molecule, and/or further studies.

In some embodiments, the system and methods disclosed herein are used to identify cancer cells from a cell population from a mammalian subject. In some aspects, the subject is a human. In some aspects, the system and methods disclosed herein are used to determine the progression of a cancer, wherein samples from a subject are obtained from two different time points and compared using the methods of the present disclosure. In some aspects, the system and methods disclosed herein are used to determine the effectiveness of an anti-cancer treatment, wherein samples from a subject are obtained before and after anti-cancer treatment and comparing the two samples using the methods of the present disclosure.

In some embodiments, the system and methods disclosed herein comprise a cancer detection system that uses a rapidly trained neural network, wherein the neural network detects cancerous cells by analyzing raw images of the cell and provides imaging information from the pixels of the images to a neural network. In some aspects, the neural network performs recognition and identification of cancerous cells using information derived from an image of the cells, among others, the area, the average intensity, the shape, the texture, and the DNA (pgDNA) of the cells. In some aspects, the neural network performs recognition of cancerous cells using textural information derived from an image of the cells, among them angular second moment, contrast, coefficient of correlation, sum of squares, difference moment, inverse difference moment, sum average, sum variance, sum entropy, entry, difference variance, difference entropy, information measures, maximal correlation coefficient, coefficient of variation, peak transition probability, diagonal variance, diagonal moment, second diagonal moment, product moment, triangular symmetry and blobness.

Non-limiting examples of cancer of interest can include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

In some embodiments, the system and methods disclosed herein can detect and/or sort circulating tumor cells or liquid tumors. In cases where the primary tumor has been previously resected or inaccessible for other reasons, a biopsy of the main tissue may not be a viable option. As such, disseminated cancer cells can be found at a much lower concentration and purity in bodily fluids, such as circulating tumor cells (CTCs) in blood, peritoneal or pleural fluids, urine, etc.

### Immune Cells

some embodiments, the system and methods disclosed herein can be utilized to isolate specific types or subtypes of immune cells. Examples of different types of immune cells can include, but are not limited to, neutrophils, eosinophils, basophils, mast cells, monocytes, macrophages, dendritic cells, natural killer (NK) cells, and lymphocytes (e.g., B cells, T cells). Additional examples of different types of immune cells can include, but are not limited to, native immune cells and engineered immune cells (e.g., engineered to express a heterologous cytokine, cytokine receptor, antigen, antigen receptor (e.g., chimeric antigen receptor or CAR), etc.). Examples of different sub-types of immune cells (e.g., T cells) can include, but are not limited to, naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Additional examples of different sub-types of immune cells can include, but are not limited to, upregulation or downregulation of one or more of the following genes: CD3, CD4, CD8, CCR7, CD45RA, CD38, HLA, CD45RO, CCR4, CD24, CD127, CCR6, CXCR3, CD24, CD38, CD19, CD19, CD20, CD27, IgD, CD14, CD16, CD56, CD11c, and CD123. For example, T cells can comprise CD38+/HLA-DR+CD4+ activated T cells or CD38+/HLA-DR+/CD8+ activated T cells. In other examples, monocytes can comprise CD16+ non-classical monocytes or CD16− classical monocytes. In another example, dendritic cells can comprise CD11c+ myeloid dendritic cells or CD123+ plasmacytoid dendritic cells. In another example, NK cells can comprise CD16+NK cells or CD16− NK cells. In some cases, an immune cell as disclosed herein may be characterized as an antibody producing cell.

In some embodiments, the system and methods disclosed herein can be utilized to isolate specific types or subtypes of T cells (e.g., CART cells) from a population of T cells. CAR T cells can be cells that have been genetically engineered to produce an artificial T-cell receptor for use in, e.g., immunotherapy. CAR T cells can be classified and sorted, using systems and methods disclosed herein, and further cultured and proliferated for the applications for, e.g., drug development.

Bacteria from Human Cells

In some embodiments, the methods disclosed herein are used for bacterial detection, wherein the human cells containing bacteria are from biological samples and are detected and tracked as they pass through the system of the present disclosure.

In some embodiments, the system and methods disclosed herein enable the acquisition of three-dimensional imaging data of bacteria present in a sample, wherein each individual bacterium is imaged from a plurality of angles. In some embodiments, the system and methods disclosed herein are used for bacterial detection, wherein the bacteria is from biological samples and are detected and tracked as they pass through the system of the present disclosure.

In some embodiments, the system and methods disclosed herein are used to detect bacteria in fluids, including blood, platelets, and other blood products for transfusion, and urine. In some aspects, the present disclosure provides a method for separating intact eukaryotic cells from suspected intact bacterial cells that may be present in the fluid sample. In some aspects, the present disclosure identifies certain bacterial species, including but not limited to: Bacillus cereus, Bacillus subtilis, Clostridium perfringens, Corynebacterium species, Escherichia coli, Enterobacter cloacae, Klebsiella oxytoca, Propionibacterium acnes, Pseudomonas aeruginosa, Salmonella choleraesuis, Serratia marcesens, Staphylococcus aureus, Staphylococcus epidermidis, Streptococcus pyogenes, and Streptococcus viridans.

In some embodiments, the system and methods disclosed herein comprise a bacterial detection system that uses a rapidly trained neural network, wherein the neural network detects bacteria by analyzing raw images of the cell and provides imaging information from the pixels of the images to a neural network. In some aspects, the neural network performs recognition and identification of bacteria using information derived from an image of the bacteria, among others, the area, the average intensity, the shape, the texture, and the DNA (pgDNA) of the cells. In some aspects, the neural network performs recognition of cancerous cells using textural information derived from an image of the cells, among them angular second moment, contrast, coefficient of correlation, sum of squares, difference moment, inverse difference moment, sum average, sum variance, sum entropy, entry, difference variance, difference entropy, information measures, maximal correlation coefficient, coefficient of variation, peak transition probability, diagonal variance, diagonal moment, second diagonal moment, product moment, triangular symmetry and blobness.

### Sepsis

In some embodiments, the system and methods disclosed herein are used for the detection and/or identification of sepsis. Without wishing to be bound by theory, plasma cells (e.g., myeloid cells such as white blood cells, lymphocytes, etc.) of a subject with hematologic bacterial infections, such as sepsis, may exhibit different morphological features (e.g., geometry, texture, shape, aspect ratio, area, etc.) than those of a subject without the hematologic bacterial infection. Thus, in some examples, the classification and sorting processes, as provided herein, may be used to diagnosis sepsis.

### Sickle Cell Disease

In some embodiments, the system and methods disclosed herein are used for the detection and/or identification of a sickle cell. In some aspects, the system and methods disclosed herein are used to image a cell and to determine if the cell is a sickle cell. The methods of the disclosure may be further used to collect the cells determined to be sickle cells. In some embodiments the cell is from a biological sample from a subject and the methods disclosed herein are used to determine whether the subject suffers from or is susceptible to a sickle cell disease. In some embodiments, the sickle cell disease is a sickle cell anemia.

### Crystals in Biological Samples

Current diagnostic methods used to detect crystals in blood and/or urine includes radiological, serological, sonographic, and enzymatic methods.

Urine crystals may be of several different types. Most commonly crystals are formed of struvite (magnesium-ammonium-phosphate), oxalate, urate, cysteine, or silicate, but may also be composed of other materials such as bilirubin, calcium carbonate, or calcium phosphate.

In some embodiments, the system and methods disclosed herein are used for the detection of crystals in biological samples. In some aspects, detected crystals are formed. In some aspects, the biological sample from a subject is imaged according to the methods described herein to determine whether the biological sample comprises a crystal. In some aspects, the biological sample is blood. In some aspects, the blood is venous blood of a subject. In some aspects, the biological sample is urine. In some aspects, the subject is a human, horse, rabbit, guinea pig, or goat. In some aspects, the methods of the disclosure may be further utilized to isolate and collect the crystal from the sample. In some aspects, the biological sample is from a subject and the system and methods of the present disclosure are used to determine whether the subject suffers from or is susceptible to disease or a condition.

In some embodiments, the methods disclosed herein are used for the analysis of a crystal from a biological sample. In some aspects, the methods disclosed herein may be used to image a crystal, and the crystal images may be analyzed for, including but not limited to, crystal shape, size, texture, morphology, and color. In some embodiments, the biological sample is from a subject and the methods disclosed herein are used to determine whether the subject suffers from a disease or a condition. In some example the subject is a human. For example, the methods of the disclosure may be used to analyze crystal in a blood sample of the human subject, and the results may be used to determine whether the subject suffers from pathological conditions, including but not limited to, chronic or rheumatic leukemia. In some aspects, the biological sample is a urine sample.

In some embodiments, the system and methods disclosed herein enable the acquisition of three-dimensional imaging data of crystals, if found in the biological sample, wherein each individual crystal is imaged from a plurality of angles.

In some embodiments, the system and methods disclosed herein comprise a crystal detection system that uses a rapidly trained neural network, wherein the neural network detects crystals by analyzing raw images of a plurality of crystals and provides imaging information from the pixels of the images to a neural network. In some aspects, the neural network performs recognition and identification of a plurality of crystals using information derived from an image of the crystals, among others, the area, the average intensity, the shape, the texture. In some aspects, the neural network performs recognition of crystals using textural information derived from an image of the cells, among them angular second moment, contrast, coefficient of correlation, sum of squares, difference moment, inverse difference moment, sum average, sum variance, sum entropy, entry, difference variance, difference entropy, information measures, maximal correlation coefficient, coefficient of variation, peak transition probability, diagonal variance, diagonal moment, second diagonal moment, product moment, triangular symmetry and blobness.

### Liquid Biopsy

A liquid biopsy comprises the collection of blood and/or urine from a cancer patient with primary or recurrent disease and the analysis of cancer-associated biomarkers in the blood and/or urine. A liquid biopsy is a simple and non-invasive alternative to surgical biopsies that enables doctors to discover a range of information about a tumor. Liquid biopsies are increasingly being recognized as a viable, noninvasive method of monitoring a patient's disease progression, regression, recurrence, and/or response to treatment.

In some embodiments, the methods disclosed herein are used for liquid biopsy diagnostics, wherein the biopsy is a liquid biological sample that is passed through the system of the present disclosure. In some aspects, the liquid biological sample that is used for the liquid biopsy is less than 5 mL of liquid. In some aspects, the liquid biological sample that is used for the liquid biopsy is less than 4 mL of liquid. In some aspects, the liquid biological sample that is used for the liquid biopsy is less than 3 mL of liquid. In some aspects, the liquid biological sample that is used for the liquid biopsy is less than 2 mL of liquid. In some aspects, the liquid biological sample that is used for the liquid biopsy is less than 1 mL of liquid. In some aspects, the liquid biological sample that is used for liquid biopsy is centrifuged to get plasma.

In some embodiments, the system and methods of the present disclosure are used for body fluid sample assessment, wherein cells within a sample are imaged and analyzed and a report is generated comprising all the components within the sample, the existence of abnormalities in the sample, and a comparison to previously imaged or tested samples from the same patient or the baseline of other healthy individuals.

In some embodiments, the system and methods of the present disclosure are used for the diagnosis of immune diseases, including but not limited to tuberculosis (TB) and acquired immune deficiency disorder (AIDS), wherein white blood cells are imaged in the system disclosed herein to examine their capacity to release pro- and anti-inflammatory cytokines.

In some embodiments, the system and methods of the present disclosure are used to assess patient immune responses to immunomodulatory therapies by imaging their white blood cells and analyzing the change in their capacity to release pro- and anti-inflammatory cytokines.

In some embodiments, the system and methods of the present disclosure are used to identify the efficacy of therapeutics and/or to guide the selection of agents or their dosage by isolating patients' white blood cells and analyzing the effect of target therapeutics on their capacity to release pro- and anti-inflammatory cytokines.

In some embodiments, the system and methods of the present disclosure are used to isolate pure samples of stem cell-derived tissue cells by obtaining images of cells, and isolating cells with desired phenotype.

### Testing Biologically Active Molecules

In some embodiments, the methods disclosed herein are used for biologically active molecule testing, for example drugs. In some embodiments, the methods of the disclosure are sued to collect desired cells from a sample and then treating the desired cells with a biologically active molecule in order to test the effect of the biologically active molecule on the collected cells.

In some embodiments, the methods and systems of the present disclosure are used for identifying the efficacy of therapeutics. In some aspects, identifying the efficacy of therapeutics using the system disclosed herein is carried out by obtaining images of a cell before and after treatment and analyzing the images to determine whether the cell has responded to the therapeutic of interest.

In some embodiments, the system and methods disclosed herein are used for diseased cell detection, wherein the diseased cells are from biological samples and are detected and tracked as they pass through the system of the present disclosure. In some aspects, the diseased cells are isolated and grouped together for further studies.

In some embodiments, the cells used in the methods disclosed herein are live cells. In some aspects, the cells that are classified as diseased cells are isolated and subsequently cultured for potential drug compound screening, testing of a biologically active molecule, and/or further studies.

Although the present disclosure has been described in certain specific aspects, many additional modifications and variations would be apparent to those skilled in the art. It is therefore to be understood that the present disclosure can be practiced otherwise than specifically described without departing from the scope and spirit of the present disclosure. Thus, some embodiments of the present disclosure should be considered in all respects as illustrative and not restrictive.

### Point-of-Care Diagnostics

Any one of the systems and methods disclosed herein (e.g., cell morphology-based classification, such as for sorting or enrichment) can be utilized for point-of-care diagnostics. A point-of-care diagnostics or point-of-care diagnostics can encompass analysis of one or more samples (e.g., biopsy samples, such as blood samples) of a subject (e.g., a patient) in a point-of-care environment, such as, for example, hospitals, emergency departments, intensive care units, primary care setting, medical centers, patient homes, a physician's office, a pharmacy or a site of an emergency. The point-of-care diagnostics as disclosed herein can be utilized to identify a pathogen (e.g., any infectious agents, gems, bacteria, virus, etc.), identify immune response in the subject (e.g., via classifying and/or sorting specific immune cell types), generate a count of cells of interest (e.g., diseased cells, healthy cells, etc.), etc.

### Point-of-Care Complete Blood Count (CBC)

CBC may provide information about types and numbers of cells in blood or plasma. White blood cell (WBC) count may be used as biomarkers for acute infection and/or inflammation. While an elevated WBC may be associated with infection, inflammation, tissue injury, leukemia and allergy, a low WBC count may be associated with viral infections, immunodeficiency, acute leukemia and bone marrow failure. Thus, an efficient point-of-care CBC may enhance (e.g., expedite) any clinical decision process that requires such information. Thus, a facility (e.g., a hospital, pharmacy, any point-of-care site, etc.) may comprise any subject embodiment of the flow cell of the present disclosure to analyze a subject's blood (or plasma) and obtain the CBC. Furthermore, the flow cell provided herein may provide CBC to track the number of WBCs before and after each treatment for a subject (e.g., chemotherapy treatment for a cancer patient). As such, in some cases, the flow cell provided herein may negate a need for hematological analysis-based CBC, which is often performed in a central or satellite laboratories.

### Computer Systems

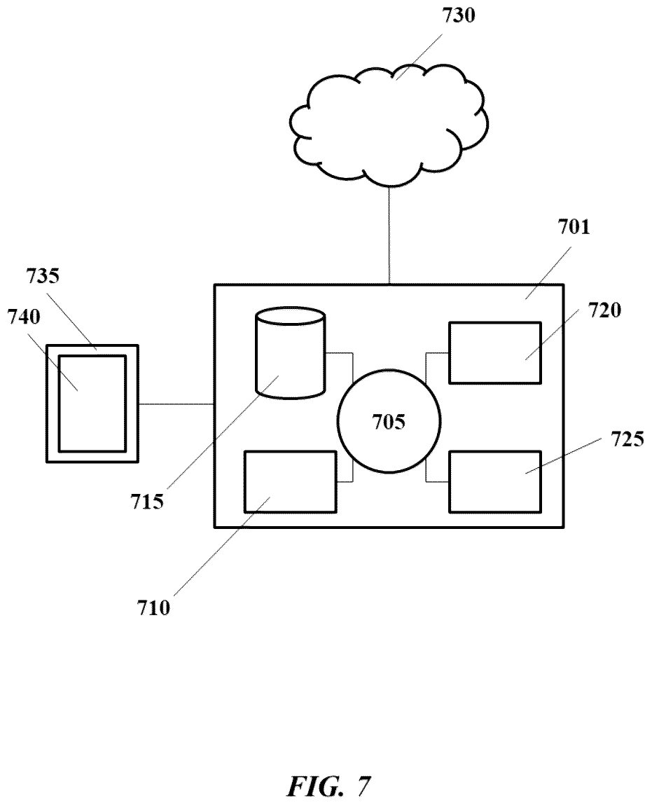

The present disclosure provides computer systems that are programmed to implement methods of the disclosure. FIG. 7 shows a computer system 701 that is programmed or otherwise configured to capture and/or analyze one or more images of the cell. The computer system 701 can regulate various aspects of components of the cell sorting system of the present disclosure, such as, for example, the pump, the valve, and the imaging device. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, the one or more images of the cell that is transported through the channel of the cell sorting system. In some cases, the computer system 701 can be configured to provide a live feedback of the images. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can be, for example, a deep learning algorithm to enable sorting of the cell.

### EXAMPLES

The following specific examples are illustrative and non-limiting. The examples described herein reference and provide non-limiting support to the various embodiments described in the preceding sections.

### Example 1. Intelligent Morphology-Based Single Cell Analysis and Sorting (iMAS)

Traditional cell classification and sorting techniques can be limited by their reliance on prior knowledge or guesswork (e.g., cell biomarkers or physical characteristics). Describes herein are systems (e.g., platforms) and methods platform that combine microfluidics, high-resolution imaging for unlabeled single cells in flow, a Convolutional Neural Network (CNN) that enables the scalable profiling and accurate classification of cells based on their morphology, and a sorting mechanism to isolate and enrich cells of interest. Models and classifiers are developed/trained to discriminate among multiple cell types, e.g., fetal nucleated red blood cells (fNRBC), non-small-cell lung carcinomas (NSCLC), hepatocellular carcinomas (HCC), and multiple subtypes of immune cells. Validation results, which include cells not used in the training data, demonstrate highly accurate cell classification: the model/classifier achieved an area under the ROC curve (AUC) metrics of >0.999 for the classification of NSCLC and HCC cell lines against a background of blood cells. Features extracted from the model/classifier have been demonstrated to provide discriminating information on cell classes for which it has not been trained, suggesting that the CNN abstracts morphological attributes that are broadly informative of the type and state of cells. Models/classifiers were trained and tuned to specific problems, and the accuracy of identifying cells of interest improved. The systems and methods disclosed herein demonstrated successful isolation of NSCLC cells from spike-in mixtures with WBCs or whole blood at concentrations as low as 1:100,000, achieving an enrichment of >25,000× on multiple cell lines, and demonstrated the enrichment of tumor-specific mutations in the sorted cells. The systems and methods disclosed herein demonstrate that deep learning applied to high-resolution cell images collected at scale can accurately classify cells in flow and can enable the label-free isolation of rare cells of interest for a wide range of applications.

### Example 2. Introduction to iMAS

High-throughput single-cell multi-omic analysis can be used to understand normal development and disease processes at cellular resolution. Single cell sequencing technologies, e.g., can allow for understanding genome, epigenome, transcriptome, or protein profile of single cells at scale. ThSuch information can provide holistic views of biological processes free of inherent biases and limitations of traditional target-based, hypothesis-driven approaches. Genotype-phenotype associations, while difficult to map, can help understanding how biological models function. However, the abovementioned analysis methods are not without challenges and failures, e.g., inadequate and qualitative (as opposed to sufficient and quantitative) description of phenotypes of cells of interest. Thus, the systems and methods of the present disclosure (e.g., iMAS) can be utilized to standardize and scale the phenotypic assessment of cells.

The systems and methods of the present disclosure can expand analysis and mapping of cells based on their phenotype. Human understanding of cell morphology can be confined within the boundaries of human language that describes it. Traditionally, “reading” cell morphology can be dependent on a cytopathologist's ability to recognize and/or physically discriminate features of individual cells (e.g., nuclear to cytoplasmic ratio, nuclear roundness, nuclear envelope smoothness, chromatin distribution, the presence of nuclear envelope grooves, etc.). However, such human-based morphological parameters can lack quantitation, thus making it difficult to be standardized. In addition, data collection in a standardized manner can bee non-trivial. Different laboratories rely on a variety of different imaging modalities. Slide preparation, staining and handling procedures can affect the analysis and contribute to challenges with standardization and repeatability. Thus, the systems and methods of the present disclosure can fulfill the unmet need of a quantitative, scalable method to collect and analyze cell morphology data, e.g., in a “big data” approach.

The systems and methods of the present disclosure can fulfill the challenges for extracting images of single cells from biological sampels (e.g., smears) introduces a multitude of challenges, such as, e.g., overlapping cells in image data that can make image segmentation complicated and/or complex, obscure angle at which the cell has been fixed on an imaging slide, etc. The systems and methods of the present disclosure can fulfill the unmet need for an image-based analysis of pathological slides.

The systems and methods of the present disclosure comprise an AI-powered morphological cell analysis and sorting platform based on high-resolution imaging of single cells in flow. The sorting capability directly connects morphology to molecular analysis at the cellular level which enables data annotation at scale in order to train and validate ultra-accurate machine learning models that can classify cells based on morphology. Disclosed herein is a continuous labeling, training, and sorting pipeline to amass a training dataset of tens of millions of annotated cells in high resolution, resulting in highly accurate classification of various cell types (and cell states). Demonstrated herein is enrichment of cell types of interest against PBMC at extreme spike-in ratios inspired by rare cell capture applications including circulating tumor cells (e.g., oncology) and circulating fetal nucleated red blood cells (e.g., prenatal diagnosis). Cells flowing through the microfluidic channel of the system can remain intact and viable at the end of the process, owing to label-free brightfield imaging and minimal cellular stress. The systems and methods of the present disclosure demonstrate the power of morphology for clustering various cell types and the potential to use the tool to profile tissue-level morphological heterogeneity akin to state-of-the-art techniques to visualize other ‘omics’ data.

### Example 3. Method and Materials

A. Microfluidics

Each chip design has a microfluidic channel height between 15 μm and 40 μm, chosen to be a few micrometers greater than the largest cells to be processed. A filter region at the input port prevents large particles, cells or cell aggregates from entering the flow channel. A buffer reagent (1X PBS) was introduced into the flow alongside the cell suspension on either side, achieving hydrodynamic focusing that keeps cells flowing at a consistent speed near the center of the flow horizontally. The flow rate used (˜0.1 m/s) is also high enough that the effects of inertial focusing are realized, confining cells to the vicinity of two vertically separated planes close to the center of the flow channel.

B. Bright-field Imaging of cells in flow

The microfluidic chip was mounted on a stage with lateral (horizontal) XY control and a fine Z control for focus. The objectives, camera, laser optics and fluidics components were all mounted on the same platform. After the microfluidic chip was loaded into the platform, it was automatically aligned and a focusing algorithm was used to bring the imaging region into the field of view. A super bright LED illumination light (SOLA SE) was directed to the imaging region, and multiple images of each cell were captured as it flowed through. Bright-field images were taken through objectives of high magnification (Leica 40×-100×) and projected onto an ultra high-speed camera. These high-resolution cell images revealed not only the cell shape and size but also finer structural features within the cytoplasm and the cell nucleus useful for discriminating cell types and states based on their morphology.

C. Software and Machine Learning

The software workload is distributed over a CPU, a GPU, and a microcontroller (MCU). The camera was periodically polled for the availability of new images. Image frames from the camera were retrieved over a dedicated 1 Gbps ethernet connection. Images were cropped to center cells within them, and the cropped images were sent to the GPU for classification by an optimized convolutional neural network (CNN) that has been trained on relevant cell categories. The CNN was based on the Inception V3 model architecture. It was written using TensorFlow and was trained using cell images annotated with their corresponding cell categories. NVidia TensorRT was used to create an optimized model which was used for inference on an NVidia GPU. The classification inference from the CNN was sent to the microcontroller, which in turn sent switching signals to synchronize the toggling of valves with the arrival of the cell at the sorting location. In order to maximize throughput, image processing happened in a parallel pipeline such that multiple cells can be in different stages of the pipeline at the same time. The primary use of the GPU was to run the optimized Convolutional Neural Network (CNN). Some basic image processing tasks such as cropping cells from the images were performed on the CPU. The CPU was also used to control all the hardware components and to read in sensor data for monitoring.

D. Data augmentation and model training

Several steps were taken to make the CNN classifier robust to imaging artifacts by systematically incorporating variation in cell image characteristics into the training data. Cells were imaged under a range of focus conditions to sample the effects of changes in focus during instrument runs. Images across four replicas of the instrument were gathered, to sample instrument-to-instrument variation. Several augmentation methods were implemented to generate altered replicas of the cell images used to train the classifier. These included standard augmentation techniques, such as horizontal and vertical flips of images, orthogonal rotation, gaussian noise, and contrast variation. Also added were salt-and-pepper noise to images to mimic microscopic particles and pixel-level aberrations. Systematic variation in the image characteristics was studied to develop custom augmentation algorithms that simulate chip variability and sample-correlated imaging artifacts on the microfluidic chip.

All cell images were resized to 299×299 pixels to make them compatible with the Inception architecture. A model comprising cell types present in normal adult blood, cell types specific to fetal blood, trophoblast cell lines, and multiple cancer cell lines drawn from NSCLC, HCC, pancreatic carcinoma, acute lymphoblastic leukemia (ALL), and acute myeloid leukemia (AML) was trained. The CNN model was also trained to detect out-of-focus images, both to use this information in auto-focusing during instrument runs and to exclude out-of-focus cell images from possible misclassification.

E. AI-Assisted Annotation of Cell Images

High-resolution images from 25.7 million cells, including cells from normal adult blood, fetal blood, trophoblast cell lines, and multiple cell lines derived from NSCLC, HCC, pancreatic carcinoma, acute lymphoblastic leukemia (ALL), and acute myeloid leukemia (AML) were collected. Images were collected by an ultra high-speed bright-field camera as cell suspensions flowed through a narrow, straight channel in a microfluidics chip. A combination of techniques were deployed in self-supervised, unsupervised, and semi-supervised learning to facilitate cell annotation on this scale. First, subject and sample source data were used to restrict the set of class labels permitted for each cell; as an example, fetal cell class annotations were disallowed in cells drawn from non-pregnant adult subjects. Next, a 64-dimensional feature vector was extracted for each cell image from a hidden layer in one of two pre-trained convolutional neural nets (CNNs) with the Inception V3 architecture: one trained on the ImageNet dataset and the other on a subset of manually labeled cell images from different image data. Following, agglomerative clustering of these feature vectors was used to divide the dataset into morphologically similar clusters which were presented for manual labeling, thereby facilitating efficient cell annotation at scale.

To further enhance the accuracy of subsequent cell classification, false positives identified from the predictions of previous trained models in an iterative manner were selectively annotated. Finally, the classes to be discriminated were balanced by feeding the harder examples of more abundant classes inspired by an active learning approach. The hard examples were identified as those that a model trained on a smaller training set has made a false inference.

F. Cell Sorting

Cell sorting was performed using built-in pneumatic microvalves on both the positive (targeted) and negative (waste) sides of the flow channel downstream of the bifurcation point. Valve timing was controlled by a DSP-based microcontroller circuit with 0.1 millisecond (ms) time precision. When the CNN inferred that a cell belongs to a targeted category, switching signals were timed to synchronize the toggling of valves with the arrival of the cell at the flow bifurcation point, and the cell flowed into a reservoir on the microfluidic chip where targeted cells are collected (also called the positive well). If the CNN infered that a cell does not belong to a targeted category, the cell flowed into a waste tube. Elliptical laser beams were focused onto both the positive and negative output channels downstream of the sorting flow bifurcation to detect passing cells and thereby monitor sorting performance in real time.

G. Blood Processing and Cell Culture

All blood samples were collected at external sites according to individual institutional review board (IRB) approved protocols and informed consent was obtained for each case. For adult control and maternal blood samples, white blood cells (WBCs) were isolated from whole blood by first centrifugation then the buffy coat was lysed with Red Blood Cell (RBC) Lysis Buffer (Roche) and then washed with PBS (Thermo Fisher Scientific). Fetal cells were isolated from fetal blood by directly lysing with the RBC lysis buffer then washed with PBS. Cells were then fixed with 4% paraformaldehyde (Electron Microscopy Sciences) and stored in PBS at 4° C. for longer term usage. A549, NCI-H1975, NCI-H23 (H23), NCI-H522 (H522), NCI-H810, Hep G2 (HEPG2), SNU-182, SNU-449, SNU-387, Hep 3B2.1-7 (HEP3B), BxPC-3, PANC-1, Kasumi-1, Reh, and HTR-8/SVneo cell lines were purchased (e.g., from ATCC).

For spike-in experiments using WBCs as mixture bases, cancer cell lines or fetal cells were first fixed with 4% paraformaldehyde and stored until mixing into WBCs. For experiments in which cell lines were spiked into whole blood, live A549 cells were first stained with CellTracker Green CMFDA (Thermo Fisher Scientific), then spiked into whole blood (EDTA) at predefined ratios (e.g., 400 or 4000 cells in 10 ml blood), followed by buffy coat RBC lysis and fixation. Prior to loading into the sorter as disclosed herein, the cell mixtures were pre-enriched by selective depletion of CD45 positive WBC cells using magnetic beads (Miltenyi). Twenty percent of the samples were saved for flow cytometry analysis to estimate the number of total cells and cancer cells before and after CD45 depletion. Based on flow cytometry analysis, the CD45 magnetic bead depletion step resulted in −11-15 fold enrichment of A549 cells.

For the isolation of human immune cells for subsequent morphological characterization, human peripheral blood mononuclear cells (PBMCs) were first isolated from whole blood by standard density gradient separation. Briefly, whole blood was diluted 1:1 with 1X PBS and layered on top of Ficoll-Paque media. Tubes were centrifuged at 400×g for 40 min at room temperature to collect the mononuclear cell fraction. Cells were then fixed with 4% PFA for 20 minutes at room temperature and washed with PBS. PBMCs were labeled with a panel of primary antibodies (CD45, CD3, CD16, CD19 and CD14) and sorted on a BD AriaII instrument for T cells (CD3+CD16/CD56−), B cells (CD3−CD16/CD56−CD14−CD19+), NK cells (CD3−CD16/CD56+) and classical monocytes (CD3−CD16/CD56−CD14+CD19−).

H. Molecular Analysis

Cell lines and WBCs of individual blood donors were genotyped with Next Generation Sequencing using a targeted SampleID panel (Swift Biosciences) that included 95 assays for exonic single nucleotide polymorphisms (SNPs) and 9 assays for gender ID. Briefly, genomic DNA was extracted from bulk cells using QIAGEN DNeasy Blood & Tissue Kit (Qiagen) and then 1 ng DNA was used as input to amplify the amplicon panels and prepare the sequencing library. For cancer cells, a panel that includes 20 assays for TP53 gene (Swift Biosciences) was pooled with the SampleID panel so cells were genotyped on both common SNPs and TP53 mutational status. From ATCC and COSMIC annotation, A549 cells are known to be TP53 wild type and NCI-H522 are known to carry a homozygous frameshift mutation (c.572_572delC). The bulk genotyping results confirmed the relative mutation status for these two cell lines.

In some experiments (e.g., integrated gradients approach), cells were retrieved from the positive outlet well of the microfluidic chip into a PCR tube, then directly lysed using Extracta DNA Prep for PCR (Quanta Bio). Cell lysates were amplified with the aforementioned Swift panels and followed by the same library preparation procedure for NGS. All libraries were sequenced on an Illumina MiniSeq instrument using MiniSeq 2×150 bp kit (Illumina).

I. Primary Sequencing Analysis and QC

Sequencing reads were aligned to the reference genome using the BWA-MEM aligner. SNP allele counts were summarized using bcftools. SNP data were subjected to quality control checks: each sample was required to have a mean coverage per SNP of >200; each SNP locus needed to have a median coverage across all samples>0.1× the median SNP to be considered; each individual SNP assay for a sample needed to have a depth of coverage>50.89 SNP assays were selected on this basis for further use in mixture analysis. Samples and individual SNP assays that failed QC were excluded from genotyping and the estimation of mixture proportions.

J. Mixture Proportion Estimation by SNP Analysis

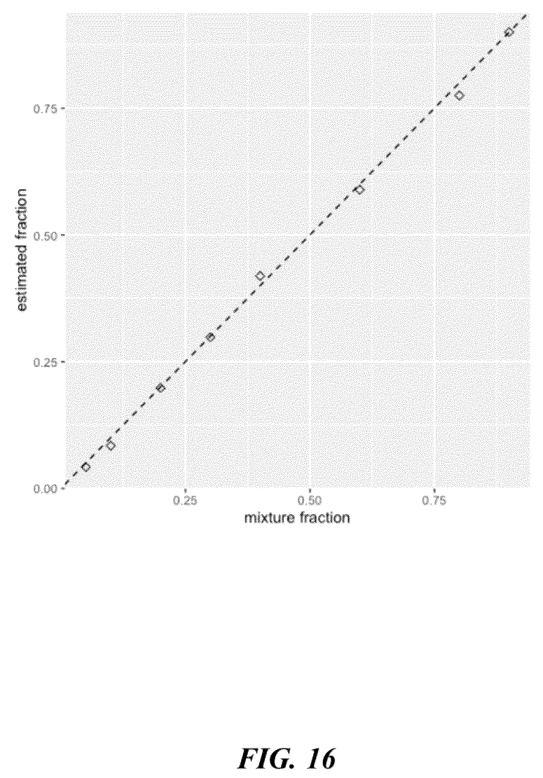

Pure diploid samples that formed the base of each mixture for spike-in experiments were clustered into the three diploid genotypes (AA, AB, BB) for each SNP using a maximum likelihood estimation that incorporated an internal estimate of error within homozygous SN. The mixture proportion of the component of interest (tumor cell line or fetal sample) was determined using maximum likelihood estimation (M_LE), in which all discrete mixture fractions in increments of 0.005 were considered (0.0, 0.005, 0.01, . . . , 1.0). For each possible mixture proportion, expected allele fractions at each SNP were determined by linearly combining the allele fractions in the two mixture components. A binomial log likelihood corresponding to each individual sample-SNP combination was computed using the expected allele fraction and an effective number of independent reads N per SNP estimated from the variance of allele fraction in mixture SNPs at which the base genotype is heterozygous (AB) and the spike-in component genotype is homozygous (AA or BB). By estimating N from the mixture data directly and using SNPs expected to have a shared allele fraction, the procedure is robust to low input for which the number of reads might exceed the number of independent molecules sampled. The overall log likelihood for each possible mixture proportion is computed as the sum of contributions from each SNP, and the mixture proportion is estimated as that at which the highest overall log likelihood is obtained. The accuracy of the procedure was verified on DNA mixtures with known composition (FIG. 16). Each composite sample contained 250 pg of DNA and the mixture proportion of DNA from the second individual was set at 5%, 10%, 20%, 30%, 40%, 60%, 80% and 90%. A close correspondence was obtained between the known mixture proportions and the SNP-based purity estimates (FIG. 16).

K. Joint Estimation of Genotypes and Sample Purity with an Expectation-Maximization (EM) Algorithm

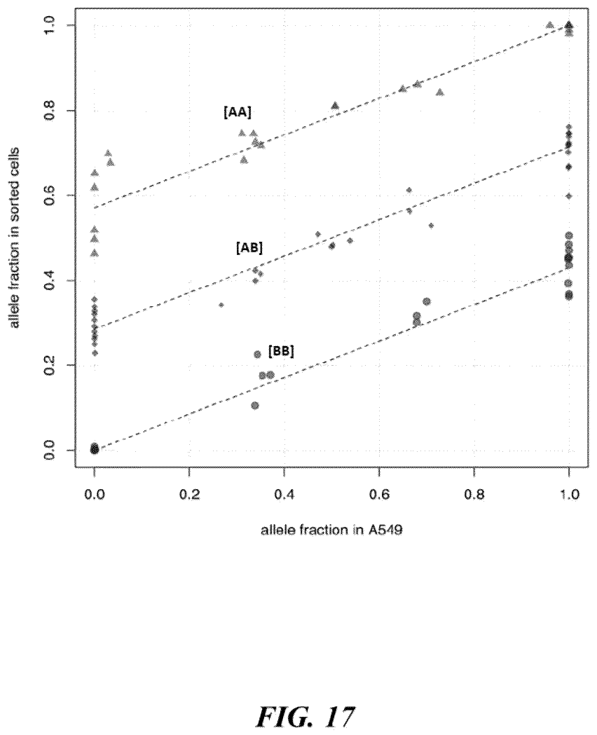

In two cases, genotypes and mixture fraction were jointly estimated from the allele fractions ϕ of SNPs in the mixture: (i) to genotype the fetal sample Fetl, which included some maternal cells in addition to fetal cells (ii) for the spike-in of A549 cells into whole blood. In each case, genotypes for one of the mixture components, designated G0, were obtained from a pure sample (from maternal DNA for the former, and from the pure A549 cell line for the latter), while the genotypes of the other sample, designated G (corresponding to the fetal sample in the former case and to the unrelated blood sample for the latter) were estimated from the data. The maternal sample was genotyped as diploid, but for pure A549, the allowed allele fractions for genotypes were 0, 1/3, 1/2, 2/3 and 1, in keeping with the known hypotriploidy of that cell line. An expectation maximization (EM) procedure was then used to jointly estimate the purity and missing genotypes. Briefly, given G and a current estimate of purity f, a binomial likelihood was estimated for each allowed missing genotype, and a maximum likelihood estimate was used to update G. Given G, a revised estimate of f was obtained by linear regression, using the expected linear relationship between the observed allele fraction ϕ and G0 over SNPs of identical G. The procedure incorporated an error rate estimate drawn from the SNPs where both components are identically homozygous. The procedure was iterated until convergence, defined as changes in the purity estimate<0.0001. Results of the EM procedure for A549 cells enriched from a starting concentration of 40 cells/ml are shown in Supplementary FIG. 17. The three dotted lines depict the linear regression used to estimate the purity given the genotypes; their slope is equal to the final purity estimate of 0.43.

L. Materials

50.8 million (M) images were gathered in order to train and validate the classifier. A dataset of 25.7M cells was imaged for the purpose of training the deep convolutional neural net: WBCs of 44 blood samples of normal adult individuals were collected which resulted in 22M cell images. Additionally, 18 fetal blood samples were collected which yielded 2.8M imaged cells. A total of 156,000 cells from four NSCLC cell lines, a total of 400,000 cells from four HCC cell lines, and another 440,000 cells from four cell lines of other types were imaged. A separate dataset of 25.1M cells from 111 samples of the cell types above were gathered in order to validate the results of the classifier. The NCI-H522 (H522) cell line was used as the sample in validation for NSCLC and Hep 3B2.1-7 (HEP3B2) for HCC respectively.

### Example 4. Platform Development

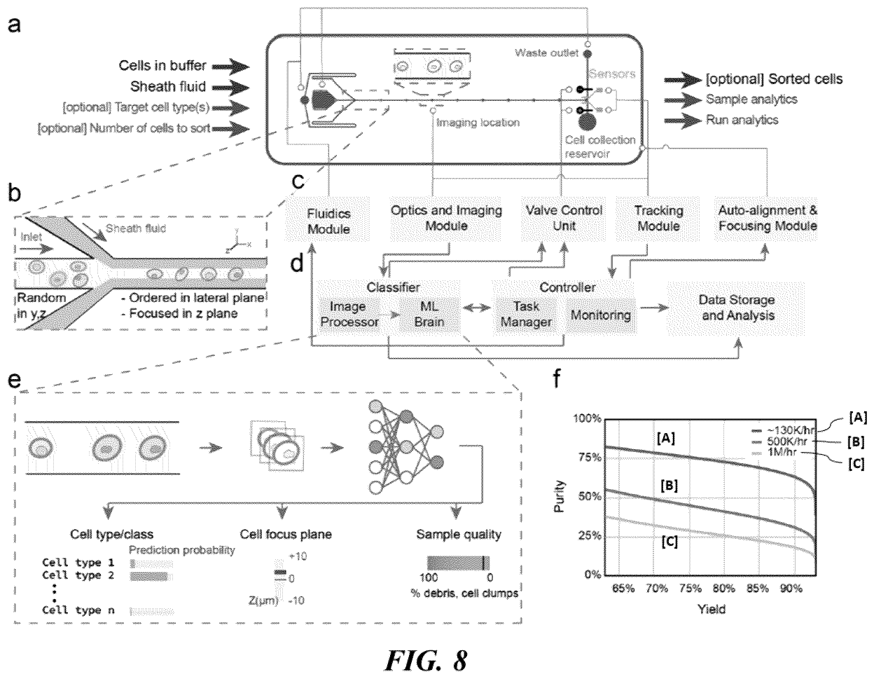

The platform as disclosed herein can allow for the input and flow of cells in suspension with confinement along a single lateral trajectory to obtain a narrow band of focus across the z-axis (FIGS. 8a-8f).

FIG. 8a shows the microfluidic chip and the inputs and output of the sorter platform of the present disclosure. Cells in suspension and sheath fluid are inputted, along with run parameters entered by the user: target cell type(s) and a cap on the number of cells to sort, if sorting is of interest. Upon run completion, the system generates reports of the sample composition (number and types of all of the processed cells) and the parameters of the run, including: length of run, number of analyzed cells, quality of imaging, quality of the sample. If sorting option is selected, it outputs isolated cells in a reservoir on the chip as well as a report of the number of sorted cells, purity of the collected cells and yield of the sort. Referring to FIG. 8b, a combination of hydrodynamic focusing and inertial focusing is used to focus the cells on a single z plane and a single lateral trajectory. Referring to FIGS. 8c and 8d, the diagram shows the interplay between different components of the software (FIG. 8c) and hardware pieces (FIG. 8d). The classifier is blown up in FIG. 8e, depicting the process of image collection, and automated real-time assessment of single cells in flow. After the images are taken, individual cell images are cropped using an automated object detection module, the cropped images are then run through a deep neural networks model trained on the relevant cells. For each image, the model generates a prediction vector over the available cell classes and an inference will be made according to a selection rule (e.g., argmax). The model may also infer the z focusing plane of the image. The percentage of debris and cell clumps may also be predicted by the neural network model as a proxy for “sample quality”. FIG. 8f shows the performance of sorting. The tradeoff between purity and yield is shown in three different modes, for profiling as sorting of 130,000, 500,000 or 1,000,000 cells within one hour

Using a combination of hydrodynamic and inertial focusing, the platform can collect ultra high-speed bright-field images of cells as they pass through the imaging zone of the microfluidic chip (FIGS. 8A and 8B). In order to capture the single cell images for processing, an automated object detection module was incorporated to crop each image centered around the cell, before feeding the cropped images into a deep convolutional neural network (CNN) based on Inception architecture, which is trained on images of relevant cell types.

In addition to classifying cells into categories of interest, the CNN was trained to assess the focus of each image (in Z plane) and identify debris and cell clusters, thus providing information to assess sample quality (FIG. 8E). A feedback loop was engineered so that the CNN inferred cell type was used in real time to regulate pneumatic valves for sorting a cell into either the positive reservoir (cell collection reservoir) for a targeted category of interest or a waste outlet (FIG. 8A). Sorted cells in the reservoir could then be retrieved for downstream processing and molecular analysis.

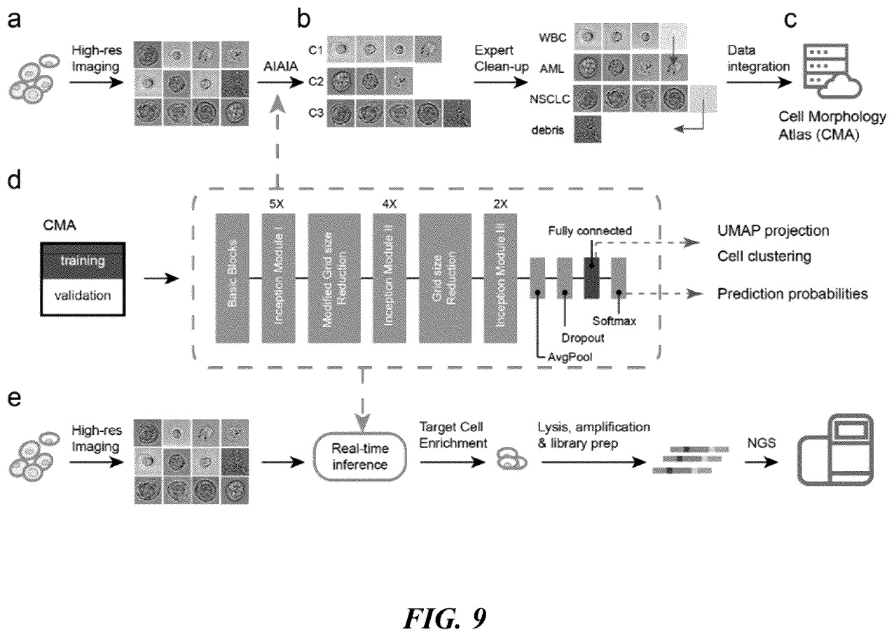

FIG. 9a shows high resolution images of single cells in flow are stored. Referring to FIG. 9b, AIAIA (AI Assisted Image Annotation) is used to cluster individual cell images into morphologically similar groups of cells. An expert uses the labeling tool to adjust and batch-label the cell clusters. In the example shown, one AML cell was mis-clustered into a group of WBC cells and an image showing a cell clump (debris) was mis-clustered in a NSCLC cell group. These errors are corrected by the “Expert clean-up” step. Referring to FIG. 9c, the annotated cells are then integrated into a Cell Morphology Atlas (CMA). Referring to FIG. 9d, the CMA is used to generate both training and validation sets of the next generation of the models. Referring to FIG. 9e, during a sorting experiment, the pre-trained model shown in FIG. 9d is used to infer the cell type (class) in real-time. The enriched cells are retrieved from the device. The retrieved cells are further processed for molecular profiling.

The platform was run in multiple different modes. In the training/validation mode FIGS. 9A-9C), the collected images of a sample were fed to the AI-Assisted Image Annotation (AIAIA), configured to use unsupervised learning to group cells into morphologically distinct sub-clusters. Using AIAIA, a user can clean up the sub-clusters by removing cells that are incorrectly clustered and annotates each cluster based on a predefined annotation schema. The annotated cell images are then integrated into the Cell Morphology Atlas (CMA), a growing database of expert-annotated images of single cells. The CMA is broken down into training and validation sets and is used to train and evaluate CNN models aimed at identifying certain cell types and/or states. Under the analysis mode (FIG. 9D), the collected images are fed into models that had been previously trained using the CMA, and a report is generated demonstrating the composition of the sample of interest. A UMAP visualization is used to depict the morphometric map of all the single cells within the sample. A set of prediction probabilities is also generated showing the classifier prediction of each individual cell within the sample belonging to every predefined cell class within the CMA. In the sorting mode (FIG. 9E), the collected images are passed to the CNN in real-time and a decision is made on the fly to assign each single cell to one of the predefined classes within the CMA. Based on the class of interest, the target cells are sorted in real-time and are outputted for downstream molecular assessment.

### Example 5. Characterization of Cell Sorter Performance

The performance of the sorter as disclosed herein was evaluated using homogeneous cell suspensions, which were prepared at a concentration of one million WBCs per milliliter. Each sample was introduced into the microfluidic chip at a flow rate of −2.2 μl/min which corresponds to a throughput of 2,160 cells per minute. A side reagent of 1×PBS buffer was simultaneously introduced with more than twice the sample flow rate to direct the cells of interest to the center of the flow stream for imaging and sorting.

A fraction (0.5%) of the cells imaged in flow were randomly selected to be sorted into the positive well of the microfluidics chip. Laser spots downstream of the bifurcation junction on either side were used to mark the passage of cells and thereby count true positive (TP), false positive (FP) and false negative (FN) sorting events. In each experiment, 50 cells out of a total of ˜10,000 imaged cells were selected for sorting, and the yield (sensitivity or recall) and purity (precision or positive predictive value) metrics were calculated as TP/(TP+FN) and TP/(TP+FP) respectively.

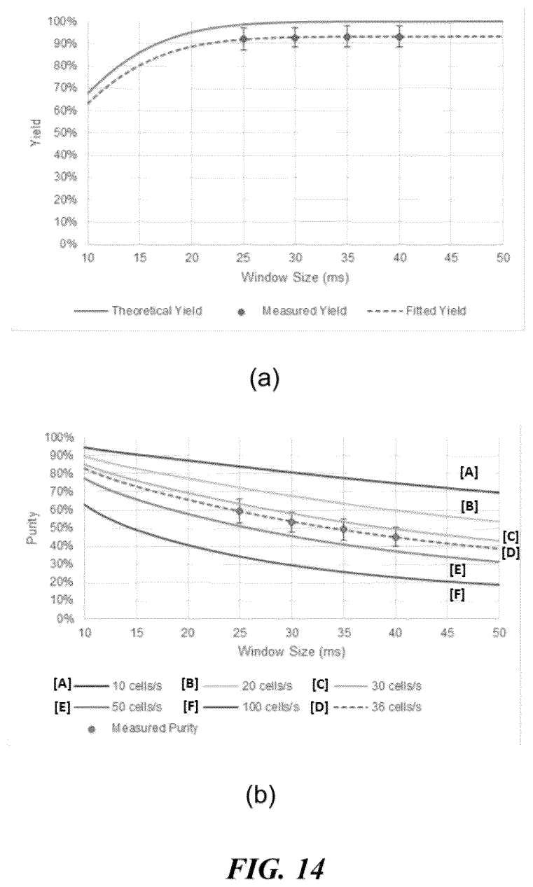

FIGS. 14a and 14b show performance of 0.5% random sorting of WBC samples using different window sizes (25, 30, 35 and 40 milliseconds). Total 341 experiments were run across 4 window sizes in 21 microfluidic devices (3 chips each from 7 photoresist mold sets) on 2 hardware systems. FIG. 14a: Yield: The theoretical curve assumes a normal distribution of cell arrival time with a standard deviation of 5 ms; fitted curve adds a limit of detection level at 93%. FIG. 14b: Purity: Solid and dotted lines are theoretical values at various cell throughput; ±3 ms exclusion zone is assumed around each cell to match measured values with the theoretical values. The error bars in both graphs represent one standard deviation (2a total) of the raw experimental data in each window size.

A key contributing factor to the trade-off between yield and purity can be the window size—the period of time for which flow is diverted toward the positive well for each sorting event. Yield and purity metrics for four different window sizes collected from 341 experimental runs are shown in FIG. 14A. For each window size, data were collected from at least 77 independent runs, distributed across 21 microfluidic devices, 7 photoresist mold sets and 2 instruments. The cell flow rate affects the number of false positives observed at any given window size and thus influences purity. The yield is not affected by the false positive rate and thus primarily depends on the window size. Theoretical curves are added to show the expected effect of changes in cell flow rate on purity, based on a normally distributed transit time for the cells with a standard deviation of 5 ms. The measured purity is closely consistent with theoretical expectation, while the yield is about 7% lower than expected. As a representative example, these results indicate that with a window size of 25 ms and a flow rate of 2,160 cells/m, the sorted cells for a rare component that constitutes 0.5% of the cells would have a yield of about 90% and a purity of about 60%. The measured data shows consistency in sorting performance across multiple microfluidic devices, instruments and runs. At a given number of cells of interest to analyze within an hour, one can adjust valve parameters (FIG. 14B) to achieve desirable purity vs yield.

### Example 6. CNN Model of Cell Morphology Classifies Diverse Cell Types with High Accuracy

The performance of the trained CNN classifier was measured on a validation dataset that included 206,673 cells from NSCLC cell lines, 76,592 cells from HCC cell lines, 192,306 cells from adult blood PBMCs, and 12,253 nucleated red blood cells (fnRBC) from fetal samples. Further, for all the cancer classes, the specific cell lines assessed in each class in the validation dataset were also distinct from those used for training.

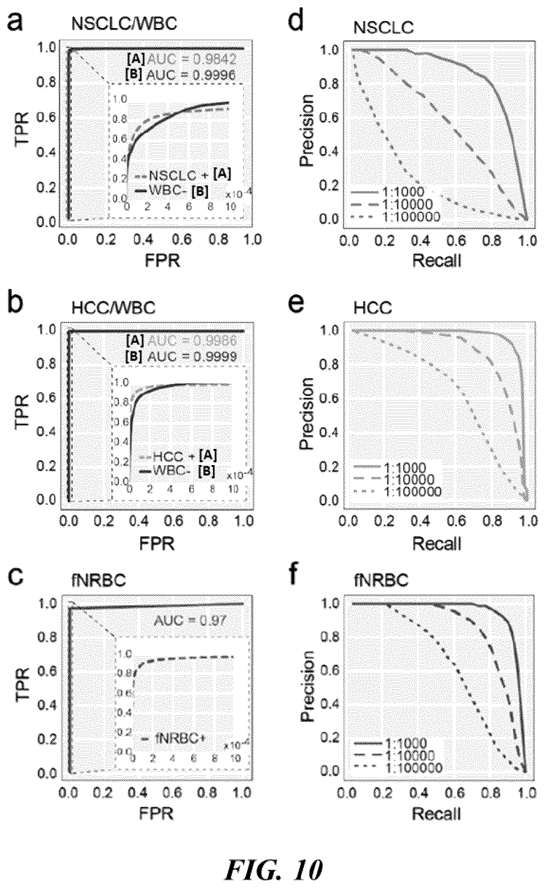

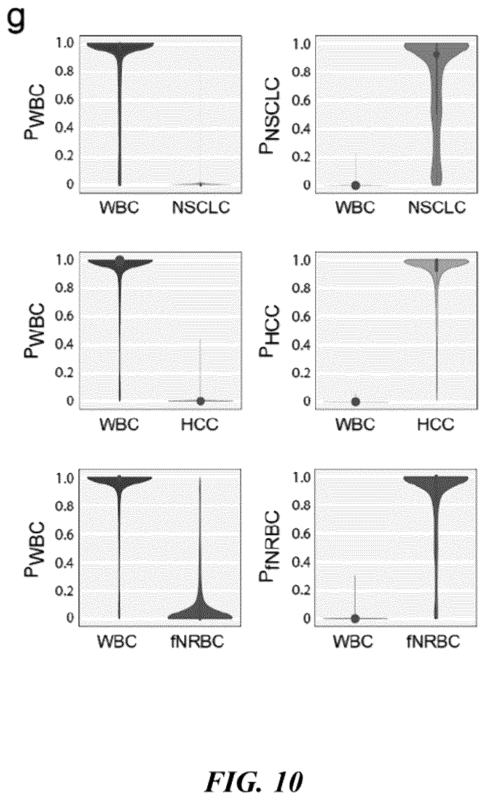

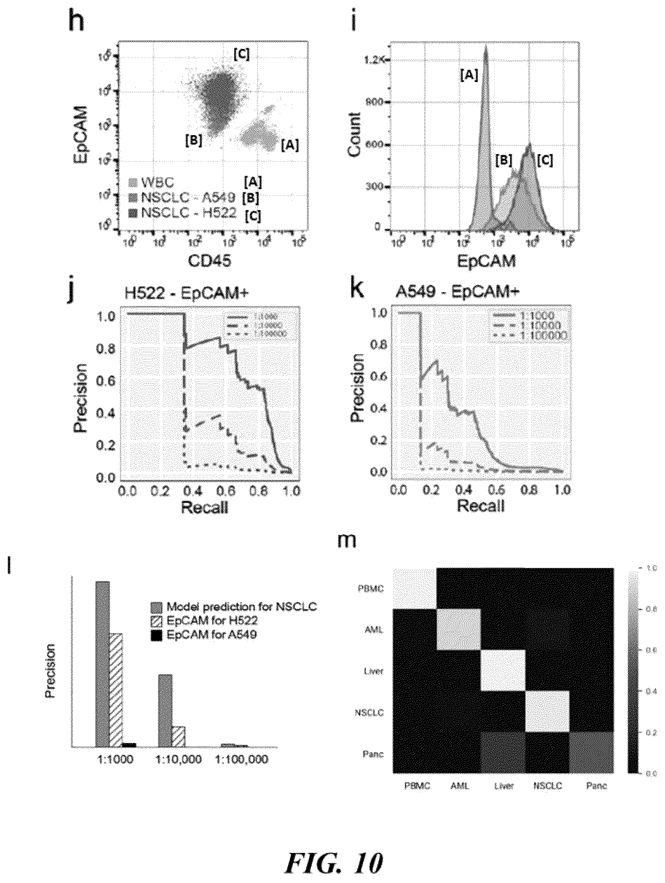

FIG. 10a shows receiver operating characteristic (ROC) curves for the classification of three cell categories—NSCLC, HCC, and fNRBC. Referring to FIGS. 10a-10c, for the cancer cell lines, two ROC curves each are shown: one for the positive selection of each category, and one for negative selection, specifically for the selection of non-blood cells. Insets zoom into the upper left portions of the ROC curves where false positive rates are very low to highlight the differences between modes of classification. AUCs achieved for NSCLC are 0.9842 (positive selection) and 0.9996 (negative selection); AUCs for HCC are 0.9986 (positive selection) and 0.9999 (negative selection); the AUC for fNRBC is 0.97 (positive selection). FIG. 10d-10f show estimated precision-recall curves at different proportions for each cell category. Precision corresponds to the estimated purity and recall to the yield of the target cells. For each cell category, three curves are shown for different target cell proportions: 1:1000, 1:10,000 and 1:100,000. FIG. 10g shows violin plots illustrating the predicted probabilities of assigning cells in each category to its appropriate class. For instance the top left plot shows the probability distribution of WBCs as well as NSCLCs being classified as WBCs (P WBC) and so on. Referring to FIGS. 10h and 10i, flow cytometry analysis shows the expression of CD45 and EpCAM in two NSCLC cell lines (A549 and H522). FIGS. 10j and 10k show precision recall plots show the performance of using EpCAM to identify NSCLC cells against PBMCs in hypothetical mixtures of 1:1000, 1:10,000 and 1:100,000. Referring to FIG. 10l (or 10L), assuming a recall of >90% is desirable, the bar graph shows the precision achievable by the model as disclosed herein versus EpCAM for identifying H522 or A549 cells against a background of WBCs at mixture ratios of 1:1000 to 1:100,000. FIG. 10m shows a heatmap representation of classifier prediction (y axis) versus actual cell classes (x axis) shows a high classifier accuracy distinguishing each pair of cells, including clear distinction between NSCLC and HCC purely.

Referring to FIGS. 10a-10c, the receiver operating characteristic (ROC) curves for three categories (NSCLC, HCC, and fNRBC) are shown. For each cell category, the area-under-curve (AUC) metric for a global assessment of classifier performance was computed. FIGS. 10d-10f show predicted precision-recall curves to also assess the expected purity and yield of the classifier for mixtures in which the ratio of cells of interest to a background of WBCs is low −1:1000, 1:10,000, or 1:100,000.

To evaluate the performance of the model on the NSCLC cell line, NCI-H522, and the HCC cell line HEP 3B2.1-7, two different strategies were tested to identify target cells: (1) positive selection (selecting the target cell class: NSCLC+ or HCC+) and (2) negative selection (selecting all non-blood cells: WBC−). The classifier performance metrics for these cell lines yielded an AUC of 0.9842 for positive selection and 0.9996 for negative selection, respectively, for the NSCLC class, and an AUC of 0.9986 and 0.9999 for positive and negative, respectively, for the HCC class (FIGS. 10a and 10b). In addition, extraordinarily low false positive rates were demonstrated for both modes of classification (FIGS. 10a and 10b insets). Although the AUC in both cases can be superior for the negative selection strategy, the positive selection strategy in both cases can enable higher yields at low false positive rates (FPR<0.0004). For fnRBCs, only the mode of positive selection was assessed, which yielded an AUC of 0.97 (FIG. 10c).

To better understand the classifier performance in supporting the reliable detection of cells of interest when they are the rare component in an in silico mixture, precision-recall curves were generated (FIGS. 10d-10f), each of which was based on positive selection. The validation results indicate that even at the most extreme dilution considered of 1:100,000, the classifier supports the detection of half the target cells with a positive predictive value (PPV) of >70% in both the fNRBC and HCC samples tested. Even for the NSCLC class, the projected PPV to detect half the present target cells is >15%. Variations in classifier performance of this magnitude are likely because cell lines of the same cancer class can have meaningful morphological differences from one another. The probability distribution of each of the classes as it relates to their identification against WBCs are also shown in FIG. 10g.

Next, the accuracy of the classifier as disclosed herein with that of the EpCAM expression was compared in identifying NSCLC and HCC cells against a background of WBCs. FIGS. 10h and 10i show the flow cytometry assessment of EpCAM and CD45 expression in the WBC population as well as A549 and H522 cells. Next, in order to estimate the performance of an approach using EpCAM expression to purify NSCLC cells, precision/recall graphs were derived from flow cytometry data (FIGS. 10j and 10k). Comparing this to FIG. 10d, one can estimate for any desirable recall, what precision the two approaches (our model versus EpCAM expression) would be able to produce. As an example, if a recall (yield) of >90% is desirable, the morphology-based classifier can be demonstrated to outperform EpCAM-based identification in different ranges of dilution (1:1000, 1:10,000 and 1:100,000) (FIG. 10l).

Also investigated was whether the classifier can identify different malignant cells against each other. FIG. 10m is the heatmap representation of classifier prediction percentages for each cell class against their actual class. This shows that morphology can be used to identify different cancer cell types against each other accurately.

### Example 7. Simultaneous Classification and Sorting for the Enrichment of Rare Cells

The simultaneous classification and enrichment of cells of two NSCLC cell lines and one fetal sample were characterized. In each case, the cells of interest were spiked into a much larger set of WBCs from a genetically distinct sample in a precisely known proportion. The fetal cells were spiked into WBCs from matched maternal blood; cells from the NSCLC cell lines were spiked into WBCs from an unrelated individual. Each mixture was then introduced into the platform as disclosed herein. Cells identified by the classifier as belonging to the class of interest (fNRBC or NSCLC) were sorted in real-time and subsequently retrieved. The two NSCLC cell lines used in these spike-in tests were A549, cell images from which were used to train the classifier, and H522, which was not used in classifier training. The two cell lines also have differing mutational profiles: A549 is known to be wildtype for TP53, an essential tumor suppressor gene, whereas NCI-H522 carries a homozygous frame-shift deletion in TP53 reported in the COSMIC database. A549 cells are also characterized by low or inconsistent EpCAM expression, suggesting that EpCAM surface marker-based enrichment would be inefficient for that cell line. EpCAM expression was assessed using flow cytometry (FIGS. 10h, 10i, and 10k).

For each spike-in mixture, the purity of the sorted cells retrieved from the system was assessed by analyzing allele fractions in a panel of SNPs. From a comparison of the known spike-in mixture proportions and the final purity, the degree of enrichment achieved for each of the samples analyzed was computed. The platform was able to achieve similar enrichment and purity for the cells of A549 and H522 (Table 1), even though the former was used to train the classifier and the latter was not. For the lowest spike-in proportion investigated of 1:100,000, purities of 19.5% and 30% were achieved for A549 and H522, corresponding to enrichments of 13,900× and 30,000× respectively.

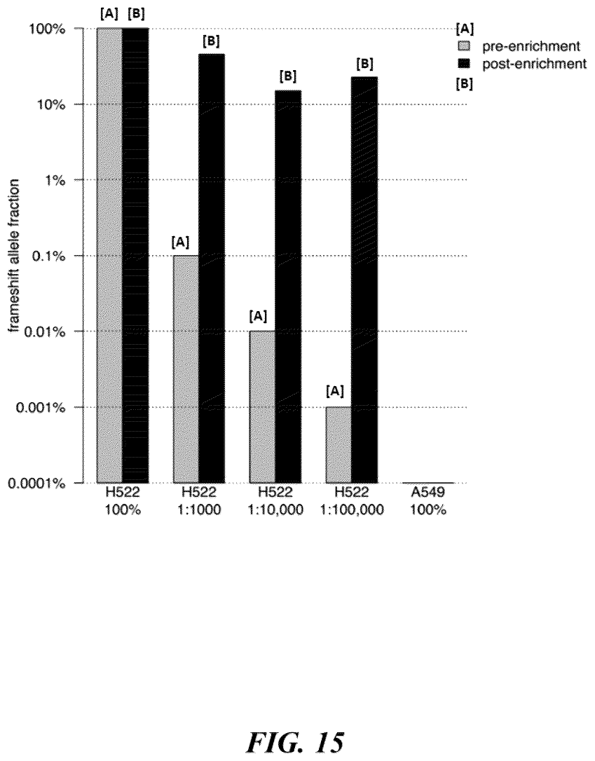

In each of the sorted cell line mixtures, also assayed was a frame-shifting single-base deletion in the TP53 gene (c.572_572delC), for which the H522 cell line is homozygous and the A549 cell line is wildtype. The proportion of the total sequence reads that contain this frame-shift mutation are shown in FIG. 15. The results are broadly consistent with estimates from the panel of SNPs depicted in Table 1. Even at the lowest starting proportion investigated of 1:100,000, it was observed that the frame-shift present at an allele fraction of 23% in the DNA extracted from the enriched cells after sorting at an allele fraction of 23%, indicating that functionally important cancer mutations can be detected even when the cells containing them are present at proportions significantly lower than the lowest explored here.

TABLE 1
Enrichment of cells spiked into WBCs at known ratios. Fet1
is a fetal blood sample spiked into cells from the corresponding
maternal sample. Cells from the A549 and H522 cell lines were
spiked into WBCs from an unrelated individual. Purity of the
enriched cells was estimated by comparing allele fractions
for a SNP panel to the known genotypes of both the cell lines
and the samples that they were spiked into.

| | Primary | | | Classifier | Sorted | Fold |
|---|---|---|---|---|---|---|
| Cell | Cell | Spike-in | Cells | Positive | Cell | Enrich- |
| Source | Class | Ratio | Imaged | Rate | Purity | ment |
| Fet1 | fNRBC | 1:1304 | 999,978 | 0.017% | 74% | 965 |
| A549 | NSCLC | 1:1000 | 69,611 | 0.150% | 62% | 348 |
| A549 | NSCLC | 1:1000 | 101,180 | 0.170% | 67% | 380 |
| A549 | NSCLC | 1:10,000 | 1,105,997 | 0.060% | 27% | 1978 |
| A549 | NSCLC | 1:10,000 | 876,421 | 0.099% | 17% | 1201 |
| A549 | NSCLC | 1:10,000 | 1,107,669 | 0.025% | 31% | 2305 |
| H522 | NSCLC | 1:10,000 | 1,050,036 | 0.030% | 26% | 2550 |
| A549 | NSCLC | 1:100,000 | 1,342,632 | 0.003% | 20% | 13,904 |
| H522 | NSCLC | 1:100,000 | 1,514,263 | 0.005% | 30% | 30,000 |
| H522 | NSCLC | 1:100,000 | 1,561,847 | 0.006% | 33% | 32,500 |

### Example 8. Enrichment of Rare Cells from Whole Blood

To mimic a liquid biopsy workflow, fluorescently-labeled live A549 cells were spiked into whole blood at concentrations of 40 cells/ml and 400 cells/ml. The spike-in cell concentrations were chosen to mimic circulating tumor cells in metastatic non-small cell lung cancer. Following, the blood samples was processed with standard buffy coat centrifugation, RBC lysis, and cell fixation. The cell mixtures were next processed with CD45 magnetic beads to remove the majority of CD45-positive WBCs for a pre-enrichment of cells of interest. The pre-enriched cells were loaded into microfluidic chips for imaging, classification, and sorting of the target A549 cells. The ratio of A549 cells to WBCs was estimated from flow analysis after the initial RBC lysis and also after CD45 depletion. The purity of the finally retrieved sorted cells was estimated by jointly analyzing allele fractions in a SNP panel in both the A549 cell line and the enriched cells. Results are shown in Table 2 for two replicates corresponding to each initial concentration. The proportion of A549 cells within the sample following CD45 depletion increased by 13× and 15× in the mixtures with 400 NSCLC cells/ml, and by 11× and 6.7× in the mixtures with 40 NSCLC cells/ml respectively. The retrieved sorted samples had final purities of 55% and 80% for the 400 cells/ml replicates, corresponding to an overall enrichment of >10,900× and >29,000× respectively) and purities of 43% and 35% for the 40 cells/ml replicates (corresponding to an overall enrichment of >33,500× and >27,800× respectively). Achievement of these high levels of purity suggests that the limit of detection for this enrichment process is likely significantly lower than the range explored.

TABLE 2
Enrichment of cells from the NSCLC cell line A549 spiked into whole blood in the
concentration 400 cells/ml or 40 cells/ml. In this case, an additional CD45 depletion
step was used to partly enrich the A549 cells prior to microfluidic sorting.

| | Percentage | Percentage | Fold | | | | |
|---|---|---|---|---|---|---|---|
| | of A549 | of A549 | Enrichment | | Classifier | Sorted | Overall |
| Spike-in Cell | after RBC | after CD45 | by CD45 | Cells | Positive | Cell | Fold |
| Concentration | lysis | [symbol] | [symbol] | Imaged | Rate | Purity | Enrichment |
| 400/ml | 0.004% | 0.06% | 13 | 1,029,175 | 0.019% | 55% | 10,900 |
| 400/ml | 0.003% | 0.06% | 16.2 | 932,665 | 0.018% | 80% | 29,000 |
| 40/ml | 0.001% | 0.01% | 11 | 949,836 | 0.007% | 43% | 33,500 |
| 40/ml | 0.001% | 0.01% | 6.7 | 1,012,315 | 0.009% | 35% | 27,800 |

[symbol]

indicates data missing or illegible when filed

### Example 9. Embeddings in the CNN Reveal Correlations Among Cells of Related Types

Having established the high degree of sensitivity and specificity of the CNN model for cell image classification in a complex mixture of cells, the correlations of both within and between cell classes were further studied.

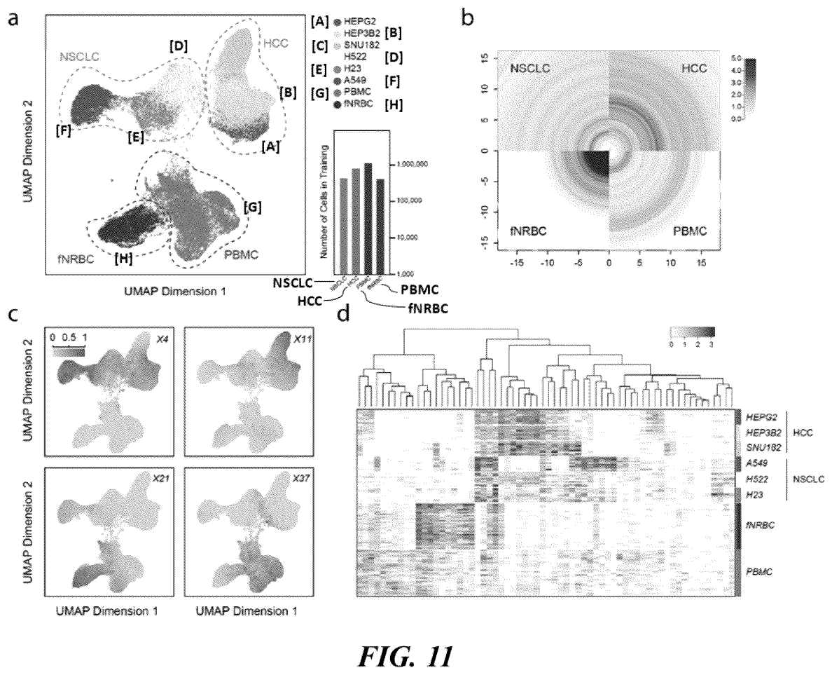

FIG. 11a shows UMAP depiction of cells sampled from classes analyzed by a CNN classifier. Each point represents a single labeled cell. Data were extracted from a 64-node fully-connected hidden layer within a convolutional neural network (CNN). Hep G2 (HEPG2), Hep 3B2.1-7 (HEP3B2) and SNU-182 (SNU182) are HCC cell lines. H522, H23 and A549 are NSCLC cell lines. fNRBCs were drawn from a pool of cells from three fetal samples, and white blood cells (WBC) were extracted from the blood of three distinct subjects. The bar chart shows the number of individual data points for each of the categories in the training set. FIG. 11b shows a heatmap of the distances of the pixels that are driving the inference decision from the center of the cell. As an example, pixels that have the highest contributions to inferring fnRBCs fall in the nucleus boundary. FIG. 11c shows heatmap representation of the fully-connected layer of the model. Each row is a single cell. Clear patterns are forming, separating different cell types. FIG. 11d shows UMAP projection of morphology profiles colored by value for the indicated dimensions.

Morphological features were extracted from a 64-node fully-connected hidden layer within the CNN and represented in UMAP with each point representing a single cell (FIG. 11a). Hep G2, Hep 3B2.1-7 and SNU-182 are HCC cell lines, of which cells from SNU-182 and Hep G2 were used to train the classifier and cells from Hep 3B2.1-7 were used to validate it. H522, H23 and A549 are NSCLC cell lines, of which A549 and H23 were used in training and H522 in validation. For comparison, fNRBCs drawn from a pool of three fetal samples, and white blood cells (WBC) extracted from the blood of three distinct adult subjects were analyzed. None of the fnRBC or WBC shown were used to train the model. The UMAP plot indicates that all of the HCC cell lines studied cluster close to one another. In contrast, the NSCLC cell lines also cluster close to one another but show greater variation, which is also reflected in the slightly lower classifier performance on H522, the cell line used in the CNN model validation. However, WBCs show a more diverse correlation structure, consistent with the existence of several morphologically variant subclasses of white blood cells.

The visualizations of cell similarity demonstrated within related samples suggest that the classifier as disclosed herein is capable of abstracting morphological features characteristic of cell classes that it has been trained on, and also that using larger and more morphologically diverse sets of representative samples for each cell category can improve and generalize model performance further,

In order to get a better understanding on what the classifier is identifying as important pixels in the images to drive the classification decisions, an attribution algorithm based on deep nets (e.g., integrated gradients algorithm) was implemented. The goal was, for example, to demonstrate which image pixels support or oppose an inference of a cell type. As shown in FIG. 11b, the distance between (i) the pixels that support the inference decision and (ii) the center of the cell within a heatmap show the degree of support or concordance over a set of 400 cells for each class.

Next, it was investigated whether there is a strong correlation between any of the features within the 64-node fully-connected hidden layer of the model and cell type. To that aim, a heatmap representation of the data was generated, as shown in FIG. 11C, with its rows showing these 64 nodes and columns being the individual cells within each sample. There are clear blocks forming within the heatmap showing signature profiles associated with PBMCs, fNRBCs and cancer cells. Within cancer cell populations, there is a clear distinction between HCC and NSCLC cell lines. Within a specific cancer cell type, some cell lines show more distinct morphological profiles. For instance, A549 shows a more unique profile compared to H522 and H23. Similarly, as also seen in the UMAP representation, SNU182 exhibits a slightly different signature compared to HEP3B2 and HEPG2 within the HCC category.

An important driver of morphological changes in cancer cells can be the epithelial-mesenchymal transition (EMT), which is an important precursor to metastasis. Several of the cell lines analyzed in the current studies have previously been investigated with respect to their EMT state. The HCC cell lines HepG2 and Hep3B can be characterized as being epithelial, while SNU-182 is seen as displaying some mesenchymal characteristics. The NSCLC cell lines H522 and H23 can be characterized as being morphologically “mesenchymal-like” and mesenchymal respectively. The EMT can be induced in A549 cells by exposure to liquids and aerosols derived from electronic cigarettes. The sampling of cell lines in the present example may be too small to firmly establish a firm morphological link to EMT status, but, without wishing to be bound by theory, a part of the variation across cell lines of the same category seen in FIG. 11a may be related to aspects of cell morphology that alter during the EMT.

Next, some of the individual features were studied more deeply. Generating the same UMAP as seen in FIG. 11a, the values of selected single features (nodes) of the fully-connected layer of the model were highlighted, as shown in FIG. 11d. As shown in FIG. 11d, there were individual dimensions that highly correlate with NSCLCs (top left), HCCs (top right), fNRBCs (bottom left), and WBCs (bottom right).

### Example 10. Embeddings in the CNN Reveal Differences Among Novel Cell Classes

To investigate whether the CNN classifier can abstract a rich enough representation of cell morphology to generalize beyond the cell classes for which it was trained, the ability of the systems and methods disclosed herein to classify and represent immune cells of known types was investigated.

Each immune cell type investigated—classical monocytes, natural killer (NK) cells, CD4 T cells, and B cells and activated CD4 T cells were obtained. For the isolation of human immune cells for subsequent morphological characterization, human peripheral blood mononuclear cells (PBMCs) were first isolated from whole blood by standard density gradient separation. Briefly, whole blood was diluted 1:1 with PBS and layered on top of Ficoll-Paque media. Tubes were centrifuged at 400×g for 40 min at room temperature to collect the mononuclear cell fraction. Cells were then fixed with 4% paraformaldehyde for 20 minutes at room temperature and washed with PBS. PBMCs were labeled with a panel of primary antibodies (e.g., CD45, CD3, CD16, CD19, and CD14) and sorted for T cells (e.g., CD3+CD16/CD56−), B cells (e.g., CD3−CD16/CD56−CD14−CD19+), NK cells (e.g., CD3−CD16/CD56+), and classical monocytes (e.g., CD3−CD16/CD56−CD14+CD19−).

For T cell activation, Human Naive CD4+ T cells were first isolated from fresh PBMCs (e.g., with EasySep Human Naive CD4+ T cell isolation kit), then cultured in RPMI medium containing 10% fetal bovine serum and 1% pen-strep, and activated by 30 U/mL IL-2 and 25 ul/1M cell CD3/CD28 dynabeads. Activated T cells were resuspended after 3-4 days in culture and beads were removed with a magnetic stand. The purity of activated T cells was measured as the CD25+/CD69+ fraction using flow cytometry and estimated to be 65% to 87%. The cell suspensions were then introduced into the microfluidic chip and imaged. Cell images were processed with a CNN that had been pre-trained on at least a subset of the CMA, as disclosed herein, but was not trained on immune cell subtypes. Cells identified as debris or out of focus were excluded from further analysis. Following, a 64-dimensional feature vector was extracted for each cell image from the penultimate hidden layer of the neural network (e.g., analogous to the procedure used to cluster cells for annotation). The first component of a principal components analysis (PCA) of the feature data was used to divide the cells into the two planes associated with flow under inertial focusing.

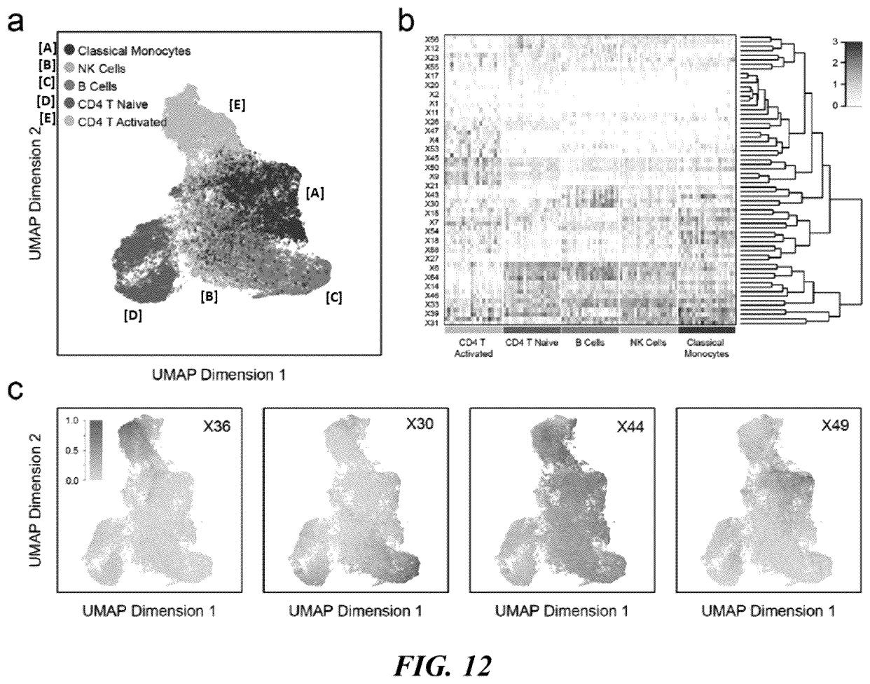

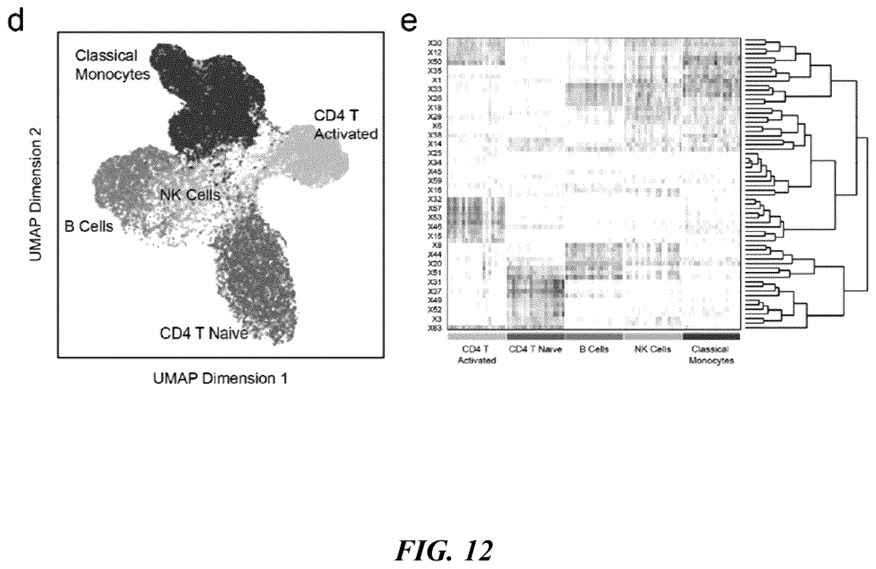

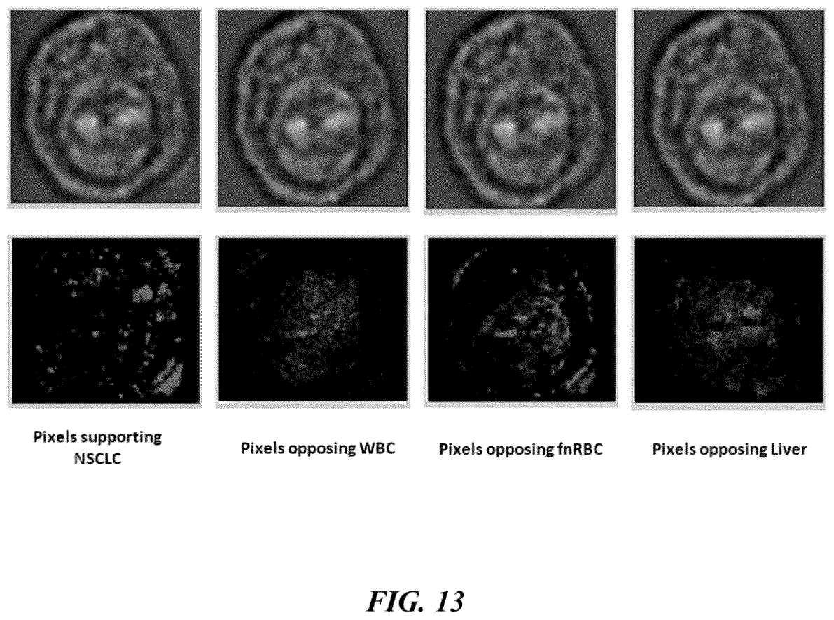

FIG. 12a shows UMAP depiction of immune cells using a CNN untrained on immune cells. Each point represents a single cell. Feature vectors were extracted from a 64-node fully-connected layer of a CNN untrained on immune cell categories. Cell categories depicted are classical monocytes, natural killer (NK) cells, CD4 T Naive cells, CD4 T Activated cells, and B cells. FIG. 12b shows heatmap depiction of immune cell subtypes. Y axis shows the different features of the fully-connected layer of the CNN. FIG. 12c shows UMAP projection of the morphology profiles colored by value for the indicated dimensions. FIG. 12d shows UMAP depiction of immune cells using a CNN that was specifically trained on immune cells. FIG. 12e shows a heatmap of the values of the CNN's 64 nodes derived from FIG. 12d.

The immune cell suspensions were then introduced into the microfluidic chip and images of the cells were collected. Cell images were processed with a CNN that had been pre-trained using the CMA, as disclosed herein, but was not trained on immune cell annotations. Cells identified as debris or out of focus were excluded from further analysis. A 64-dimensional feature vector for each cell image was then extracted from the penultimate hidden layer of the neural network, analogous to the procedure used to cluster cells for annotation. The first component of a principal components analysis (PCA) of the feature data was used to divide the cells into the two planes associated with flow under inertial focusing. A UMAP visualization of these morphological feature vectors for one of the flow planes is depicted in FIG. 12a. Points close to one another on the plot indicate a similarity in morphology as viewed through the lens of the untrained CNN. In this visualization, classical monocytes, CD4 T Naive cells and CD4 T Activated cells are seen to clearly cluster separately from NK cells and B cells, which are seen to have overlapping but differing morphological distributions. UMAP projection of the morphological features for specific dimensions (FIG. 12c) shows strong correlation between single dimensions and specific subtypes.

Thus, it was found that CNNs trained on a diverse set of cell types develop a rich representation of the space of cell morphologies that allow them to distinguish between cell types on which they have not been explicitly trained. This is encouraging for the development of unsupervised approaches for the classification of novel cell types and states by morphological characteristics in a variety of applications. These data also suggest that with the collection of more annotated cell images, a model could likely be trained on immune cells that would achieve superior classification performance.

### Example 11. CNN Tuned to Specific Problems has Improved Accuracy

Next, it was investigated if training a CNN including the immune cell subtypes can effect better separation between these subtypes. Similar to as described above, classical monocytes, natural killer (NK) cells, CD4 T Naive and activated cells, and B cells were generated from a specific subset of donor samples. The images were taken with the microfluidic system/platform as disclosed herein and used as training set for an immune-cell CNN. Same cell samples were created from a separate group of donor samples for validation. FIG. 12d is the UMAP projection of the 64-dimensional feature vector of the validation set from this immune-cell CNN. Similar to above, the first component of a PCA of the feature data was used to correct for the two planes associated with inertial focusing.

Compared to the model that was never trained on immune cell subtypes (FIG. 12a), the pre-trained model generates better separation between the immune cell subtypes. Since this model has been purely trained on the immune cells, there are more features in the 64-dimensional feature vector that specifically contribute to identifying one subtype versus another (FIG. 12e) compared to a model that was trained on all other cell types (FIG. 12b). It is remarkable that in a model that had never been trained on these subtypes with minute morphological differences, still a unique signature is visible as depicted in the heatmap representation (FIG. 12b).

### Example 12. Integrated Gradients Approach

Also implemented was Integrated Gradients, an approach to demonstrate which image pixels can support or oppose an inference of a cell type. The idea was based on a smart variation of calculating the gradients of the inferred class probability with respect to the image pixels in a way that may preserve several natural axioms. The pixels maximizing the magnitude of the gradient were determined to be important pixels and the sign can determine whether the pixels may support or oppose the inference. Both pixels that support the inferred cell type and the pixels that oppose other cell types were analyzed. FIG. 13 demonstrates an example pf a NSCLC cell probed against WBC, fnRBC, and Liver carcinoma cell types. The model can look at both nuclear and cytoplasmic features along with the pixels that indicate size and the shape of the cell membrane. The cell can have a double nucleoli which seems to be observed by the pixels supporting NSCLC inference. The model disclosed herein can perform a through sweep of other nuclear features and also cytoplasmic features, e.g., vacuoles and chromatin patterns.

### EMBODIMENTS

The following non-limiting embodiments provide illustrative examples of the invention, but do not limit the scope of the invention.

Embodiment 1. In an aspect, the present disclosure provides a method comprising:
(a) obtaining image data of a plurality of cells, wherein the image data comprises tag-free images of single cells;
(b) processing the image data to generate a cell morphology map, wherein the cell morphology map comprises a plurality of morphologically-distinct clusters corresponding to different types or states of the cells;
(c) training a classifier using the cell morphology map; and
(d) using the classifier to automatically classify a cellular image sample based on its proximity, correlation, or commonality with one or more of the morphologically-distinct clusters,
optionally wherein:
   (1) each cluster of the morphologically-distinct clusters is annotated based on a predefined annotation schema; and/or
   (2) the classifier is configured to automatically classify the cellular image sample, without requiring prior knowledge or information about a type, state, or characteristic of one or more cells in the cellular image sample; and/or
   (3) the cell morphology map is generated based on one or more morphological features from the processed image data; and/or
   (4) the cell morphology map comprises an ontology of the one or more morphological features; and/or
   (5) the one or more morphological features are attributable to unique groups of pixels in the image data; and/or
   (6) the image data is processed using a machine learning algorithm to group the single cell images into the plurality of morphologically-distinct clusters; and/or
   (7) the machine learning algorithm is configured to extract the one or more morphological features from each cell of the single cells; and/or
   (8) the machine learning algorithm is based on unsupervised learning; and/or
   (9) processing the image data further comprises annotating each cluster of the morphologically-distinct clusters to generate annotated cell images belonging to said each cluster of the morphologically-distinct clusters; and/or
   (10) an interactive annotation tool is provided that permits one or more users to curate, verify, edit, and/or annotate the morphologically-distinct clusters; and/or
   (11) the interactive annotation tool permits the one or more users to annotate each cluster using a predefined annotation schema; and/or
   (12) the interactive annotation tool permits the one or more users to exclude cells that are incorrectly clustered; and/or
   (13) the interactive annotation tool permits the one or more users to exclude debris or cell clumps from the clusters; and/or
   (14) the interactive annotation tool permits the one or more users to assign weights to the clusters; and/or
   (15) the interactive annotation tool is provided on a virtual crowdsourcing platform to a community comprising of the one or more users; and/or
   (16) the classifier is useable on both known or unknown populations of cells in a sample; and/or
   (17) one or more of the clusters comprises sub-clusters; and/or
   (18) two or more of the clusters overlap.

Embodiment 2. An aspect of the disclosure provides a method comprising:
(a) processing a sample and obtaining cellular image data of the sample;
(b) processing the cellular image data to identify one or more morphological features that are potentially of interest to a user; and
(c) displaying, on a graphical user interface (GUI), a visualization of patterns or profiles associated with the one or more morphological features,
optionally wherein:
   (1) the image data is processed using a cell morphology map, wherein the cell morphology map comprises a plurality of morphologically-distinct clusters corresponding to different types or states of cells; and/or
   (2) the GUI permits the user to select one or more of the morphological features to base sorting of the sample; and/or
   (3) the GUI permits the user to select one or more regions of the map having the one or more morphological features; and/or
   (4) the GUI permits the user to select the one or more regions by using an interactive tool to draw a bounding box encompassing the one or more regions; and/or
   (5) the bounding box is configured having any user-defined shape and/or size; and/or
   (6) the method further comprises receiving an input from the user via the GUI, wherein the input comprises the user's selection of the morphological feature(s) or clusters of the map; and/or
   (7) the method further comprises sorting a group of cells from the sample, the group of cells possessing the selected morphological feature(s); and/or
   (8) the one or more morphological features are identified to be potentially of interest to the user based on a set of criteria input by the user to the GUI; and/or
   (9) the one or more morphological features are identified to be potentially of interest to the user based on one or more previous sample runs performed by the user; and/or
   (10) the one or more morphological features are identified to be potentially of interest to the user based on a research objective of the user; and/or
   (11) the one or more morphological features are identified from the cellular image data within less than one minute of processing the sample; and/or
   (12) the one or more morphological features are identified from the cellular image data within less than five minutes of processing the sample; and/or
   (13) the one or more morphological features are identified from the cellular image data within less than ten minutes of processing the sample.

Embodiment 3. An aspect of the disclosure provides a cell analysis platform comprising:
a cell morphology atlas (CMA) comprising a database having a plurality of annotated single cell images that are grouped into morphologically-distinct clusters corresponding to a plurality of predefined cell classes;
a modeling library comprising a plurality of models that are trained and validated using datasets from the CMA, to identify different cell types and/or states based at least on morphological features; and
an analysis module comprising a classifier that uses one or more of the models from the modeling library to (1) classify one or more images taken from a sample and/or (2) assess a quality or state of the sample based on the one or more images,
optionally wherein:
   (1a) each cluster comprises a population of cells that exhibits one or more common or similar morphological features; and/or
   (1b) each population of cells is of a same cell type or of different cell types; and/or
   (1c) the one or more images depict individual single cells; and/or
   (1d) the one or more images depict clusters of cells; and/or
   (1e) the sample comprises a mixture of cells; and/or
   (1f) the quality or state of the sample is assessed at an aggregate level; and/or
   (1g) the quality or state of the sample is indicative of a preparation or priming condition of the sample; and/or
   (1h) the quality or state of the sample is indicative of a viability of the sample; and/or
   (2a) the platform comprises a tool that permits a user to train one or more models from the modeling library; and/or
   (2b) the tool is configured to determine a number of labels and/or an amount of data that the user needs to train the one or more models, based on an initial image dataset of a sample provided by the user; and/or
   (2c) the number of labels and/or the amount of data are determined based at least on a degree of separability between two or more clusters that the user is interested in differentiating using the one or more trained models; and/or
   (2d) the number of labels and/or the amount of data are further determined based at least on a variability or differences in morphological features between the two or more clusters; and/or
   (2e) the tool is configured to determine and notify the user if additional labels and/or additional data is needed to further train the one or more models for improving cell classification, or for improving differentiation between two or more cell types or clusters; and/or
   (2f) the tool is configured to allow the user to customize the one or more models to meet the user's preferences/needs; and/or
   (2g) the tool is configured to allow the user to combine or fuse together two or more models; and/or
   (3a) the plurality of models are configured and used to discriminate among and between multiple different cell types; and/or
   (3b) the multiple different cell types comprise fNRBC, NSCLC, HCC, or multiple subtypes of immune cells; and/or
   (3c) the plurality of models are configured to abstract morphological attributes/features/characteristics that are associated and indicative of a type and/or state of the cells; and/or
   (3d) the classifier is capable of providing discriminating information about new cell classes that are not present in the CMA and for which the plurality of models have not been trained on; and/or
   (3e) the plurality of models are validated to demonstrate accurate cell classification performance, having a high degree of sensitivity and sensitivity as characterized by an area under receiving operating characteristic (ROC) curve (AUC) metric of greater than about 0.97 in identifying one or more target cells; and/or
   (3f) the classifier is capable of identifying and discriminating target cells at dilution concentrations ranging from 1:1000 to 1:100,000; and/or
   (3g) the classifier is capable of distinguishing between different sub-classes of malignant cells; and/or
   (3h) the classifier is configured to generate a set of prediction probabilities comprising a prediction probability of each individual cell within the sample belonging to each predefined cell class within the CMA; and/or
   (3i) the set of prediction probabilities is provided as a prediction vector over the available cell classes within the CMA; and/or
   (3j) the analysis module is configured to assign each single cell to one of the predefined classes within the CMA based on the set of prediction probabilities; and/or
   (3k) one or more of the models is configured to assess the quality of the sample based on an amount of debris or cell clumps detected from the one or more images; and/or
   (3l) one or more of the models is configured to assess the quality of the sample based on a ratio of live/viable cells to dead/damaged cells; and/or
   (3m) the plurality of models comprise one or more deep neural network models; and/or
   (3n) the one or more deep neural network models comprise convolutional neural networks (CNNs); and/or
   (3o) the plurality of models in the modeling database are continuously trained and validated as new morphologically-distinct clusters are being identified and added to the CMA; and/or
   (3p) the clusters in the CMA are mapped to one or more cellular molecular profiles based on genomics, proteomics, or transcriptomics; and/or
   (3q) the mapping is used to identify or develop new molecular markers; and/or
   (4a) the analysis module comprises an interface that permits a user to customize and select which model(s) from the modeling database to use in the classifier; and/or
   (4b) the platform further comprises a reporting module that is configured to generate a report showing a cellular composition of the sample based on results obtained by the analysis module; and/or
   (4c) the report comprises a visualization depicting a morphometric map of all single cells within the sample; and/or
   (4d) the visualization comprises a uniform manifold approximation and projection (UMAP) graph; and/or
   (4e) the visualization comprises a multi-dimensional morphometric map in three or more dimensions; and/or
   (4f) the report comprises a heatmap representation of classifier prediction percentages for each cell class against the actual cell class; and/or
   (4g) the heatmap representation displays correlations between one or more extracted features and individual cell types; and/or
   (4h) the plurality of models comprise a neural network, and the extracted features are extracted from a hidden layer of the neural network; and/or
   (5a) the platform further comprises a sorting module that is configured to sort the cells in the sample substantially in real-time, based on one or more classes of interest input by a user; and/or
   (5b) the sorted cells are collected for downstream molecular assessment/profiling; and/or
   (6a) the sample comprises two or more test samples, and wherein the analysis module is configured to determine a morphological profile for each test sample; and/or
   (6b) the analysis module is further configured to compare the morphological profiles between the two or more test samples; and/or
   (6c) a comparison of the morphological profiles is used to evaluate a response of each test sample after the test samples have been contacted with a drug candidate; and/or
   (6d) a comparison of the morphological profiles is used to differentiate responses of the test samples after the test samples have been contacted with different drug candidates; and/or
   (6e) a comparison of the morphological profiles is used to determine a degree or rate of cell death in each test sample; and/or
   (6f) a comparison of the morphological profiles is used to determine a degree or rate of cell stress or damage in each test sample; and/or
   (6g) a comparison of the morphological profiles is used to determine whether a test sample is treated or untreated; and/or
   (7a) the platform provides an inline end-to-end pipeline solution for continuous, labeling and sorting of multiple different cell types; and/or
   (7b) the CMA is scalable, extensible and generalizable to incorporate new clusters of morphologically-distinct cells and/or new models; and/or
   (7c) the modeling library is scalable, extensible and generalizable to incorporate new types of machine learning models; and/or
   (7d) the analysis module is configured to detect correlations between new clusters and existing clusters of cells in the CMA; and/or
   (7e) one or more of the models in the modeling library are removable or replaceable with new models.

Embodiment 4. An aspect of the disclosure provides a method comprising:
(a) obtaining image data of a plurality of cells, wherein the image data comprises images of single cells captured using a plurality of different imaging modalities;
(b) training a model using the image data; and
(c) using the model with aid of a focusing tool to automatically adjust in real-time a spatial location of one or more of cells in a sample within a flow channel as the sample is being processed,
optionally wherein:
   (1) the model is used to classify the one or more cells, and wherein the spatial location of the one or more of cells is adjusted based on a cell type; and/or
   (2a) the image data comprises in-focus images of the cells; and/or
   (2b) the image data comprises out-of-focus images of the cells; and/or
   (2c) the in-focus and out-of-focus images are captured under a range of focus conditions to sample the effects of changes in focus during processing of samples; and/or
   (2d) the image data comprises bright field images of the cells; and/or
   (2e) the image data comprises dark field images of the cells; and/or
   (2f) the image data comprises fluorescent images of stained cells; and/or
   (2g) the image data comprises color images of the cells; and/or
   (2h) the image data comprises monochrome images of the cells; and/or
   (2i) the model comprises a cell morphology map based on the different imaging modalities; and/or
   (3a) the image data comprises images of the single cells captured at a plurality of locations along the flow channel; and/or
   (3b) the plurality of locations are located on different planes within the flow channel; and/or
   (3c) the different planes are located on a vertical axis; and/or
   (3d) the different planes are located on a horizontal axis; and/or
   (3e) the different planes are located on a longitudinal axis of the flow channel; and/or
   (3f) the plurality of locations are located on a same plane within the flow channel; and/or
   (3g) the image data comprises images of the single cells captured at different angles; and/or
   (3h) the image data comprises images of the single cells captured from different perspectives within the flow channel; and/or
   (4a) the image data is annotated with one or more of the different imaging modalities prior to training the model; and/or
   (4b) each image in the image data is annotated with its corresponding location in the flow channel; and/or
   (4c) the location in the flow channel is defined as a set of spatial coordinates; and/or
   (4d) each image in the image data is marked with a timestamp; and/or
   (4e) each image in the image data is annotated with a cell type or state; and/or
   (5a) the method further comprises generating altered replicas of one or more images in the image data prior to training the model; and/or
   (5b) the altered replicas are generated using one or more augmentation techniques comprising horizontal or vertical image flips, orthogonal rotation, gaussian noise, contrast variation, or noise introduction to mimic microscopic particles or pixel-level aberrations; and/or
   (6a) the focusing tool utilizes hydrodynamic focusing and inertial focusing; and/or
   (6b) the model and the focusing tool are used to focus the one or more cells in the sample on a single Z-plane and a single lateral trajectory along the flow channel; and/or
   (6c) the method further comprises using the model with aid of one or more microfluidic elements to automatically adjust in real-time a velocity of the one or more cells in the sample within the flow channel as the sample is being processed; and/or
   (6d) the one or more microfluidic elements comprise valves and pumps; and/or
   (6e) the model is used to classify the one or more cells, and wherein the velocity of the one or more of cells is adjusted based on a cell type.

Embodiment 5. An aspect of the disclosure provides a method comprising:
(a) obtaining image data of a plurality of cells, wherein the image data comprises images of single cells captured under a range of focal conditions;
(b) training a model using the image data;
(c) using the model to assess a focus of one or more images of one or more of cells in a sample within a flow channel as the sample is being processed; and
(d) automatically adjusting in real-time an imaging focal plane based on the image focus assessed by the model,
optionally wherein:
   (1) the model is used to classify the one or more cells, and wherein the imaging focal plane is adjusted based on a cell type; and/or
   (2) the range of focal conditions comprise in-focus and out-of-focus conditions; and/or
   (3) the imaging focal plane is automatically adjusted to bring subsequent images of the one or more cells into focus; and/or
   (4) the imaging focal plane is automatically adjusted to enhance a clarity of subsequent images of the one or more cells; and/or
   (5) the imaging focal plane is adjusted to focus on different portions of the one or more cells; and/or
   (6) the different portions comprise an upper portion, a mid portion, or a lower portion of the one or more cells.

Embodiment 6. An aspect of the disclosure provides a method comprising:
(a) obtaining image data of a plurality of cells, wherein the image data comprises images of single cells captured using a plurality of different imaging modalities;
(b) training an image processing tool using the image data; and
(c) using the image processing tool to automatically identify, account for, and/or exclude artifacts from one or more images of one or more cells in a sample as the sample is being processed, optionally wherein:
   (1) the different imaging modalities systematically incorporate or induce variations in cell image characteristics into the image data that is used to train the image processing tool; and/or
   (2) the artifacts are due to non-optimal imaging conditions during capture of the one or more images; and/or
   (3) the non-optimal imaging conditions include lighting variability and/or oversaturation; and/or
   (4) the non-optimal imaging conditions are induced by external factors including vibrations, misalignment or power surges/fluctuations; and/or
   (5) the artifacts are due to degradation of an imaging light source; and/or
   (6) the artifacts are due to debris or defects in an optics system; and/or
   (7) the artifacts are due to debris or clumps that are inherent in the sample; and/or
   (8) the artifacts are due to debris or unknown objects within a system that is processing the sample; and/or
   (9) the artifacts are due to deformation changes to a microfluidics chip that is processing the sample, wherein the deformation changes comprise shrinkage or swelling of the chip; and/or
   (10) the image processing tool is configured to compare (a) the one or more images of the one or more cells in the sample to (b) a set of reference images of cells within same or similar locations within the flow channel, to determine differences between the one or more images and the set of reference images; and/or
   (11) the image processing tool is configured to edit the one or more images to account or correct for the differences; and/or
   (12) the image processing tool is configured to assign weights to the differences.

Embodiment 7. An aspect of the disclosure provides an online crowdsourcing platform comprising:
a database storing a plurality of single cell images that are grouped into morphologically-distinct clusters corresponding to a plurality of predefined cell classes;
a modeling library comprising one or more models; and
a web portal for a community of users, wherein the web portal comprises a graphical user interface (GUI) that allows the users to (1) upload, download, search, curate, annotate, or edit one or more existing images or new images into the database, (2) train or validate the one or more models using datasets from the database, and/or (3) upload new models into the modeling library,
optionally wherein:
   (1) the one or more models comprise machine learning models; and/or
   (2) the web portal is configured to permit the users to buy, sell, share or exchange one or more models with one another; and/or
   (3) the web portal is configured to generate incentives for the users to update the database with new annotated cell images; and/or
   (4) the web portal is configured to generate incentives for the users to update the modeling library with new models; and/or
   (5) the web portal is configured to permit the users to assign ratings to annotated images in the database; and/or
   (6) the web portal is configured to permit the users to assign ratings to the models in the modeling library; and/or
   (7) the web portal is configured to permit the users to share cell analysis data with one another; and/or
   (8) the web portal is configured to permit the users to create an ontology map of various cell types and/or states.

Embodiment 8. An aspect of the disclosure provides a method of identifying a disease cause in a subject, the method comprising:
(a) obtaining a biological sample from the subject;
(b) suspending the sample into a carrier, to effect constituents of the biological sample to (i) flow in a single line and (ii) rotate relative to the carrier;
(c) sorting the constituents into at least two populations based on at least one morphological characteristic that is identified substantially concurrently with the sorting of the constituents; and
(d) determining a disease cause of the subject as indicated by at least one population of the at least two populations,
optionally wherein:
   (1) the constituents are regularly spaced in the single line; and/or
   (2) the carrier comprises a housing that encloses at least the constituents of the biological sample, and wherein the constituents are rotating relative to the housing; and/or
   (3) the disease cause is a pathogen, and wherein the at last one population comprises the pathogen; and/or
   (4) the method further comprises sequencing at least a portion of a genome of the pathogen; and/or
   (5) the pathogen is a virus; and/or
   (6) the disease cause is indicated by a comparison between (i) a number of the constituents in the at least one population and (ii) a number of the constituents in a different population of the at least two populations; and/or
   (7) the disease cause is indicated by sequence information of the at least one population; and/or
   (8) the at least one population comprises antibody producing cells; and/or
   (9) the at least one population comprises immune cells; and/or
   (10) the constituents comprise a plurality of cells; and/or
   (11) the at least one morphological characteristic is identified by analyzing one or more images of the constituents prior to or substantially concurrently with the sorting; and/or
   (12) the at least one morphological characteristic comprises a plurality of morphological characteristics; and/or
   (13) the constituents of the biological sample are label-free; and/or
   (14) the image data is processed using a machine learning algorithm to group the single cell images into the plurality of morphologically-distinct clusters.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-163. (canceled)

164. A method, comprising:
generating a cell morphology map using image data comprising tag-free images of a plurality of single cells, wherein the cell morphology map comprises a plurality of morphologically-distinct clusters corresponding to different types or states of the cells; and
using a classifier to automatically classify a cellular image sample using a proximity, a correlation, a commonality, or a combination thereof, with one or more of the morphologically-distinct clusters, the classifier having been trained using the cell morphology map.

165. The method of claim 164, further comprising: annotating each morphologically-distinct cluster of the morphologically-distinct clusters using at least a predefined annotation schema.

166. The method of claim 164, further comprising: automatically classifying, using the classifier, the cellular image sample, without prior knowledge or information about a type, state, or characteristic of one or more cells of the cellular image sample.

167. The method of claim 164, wherein the cell morphology map is generated using at least one or more morphological features from the image data, wherein the cell morphology map comprises an ontology of the one or more morphological features.

168. The method of claim 167, wherein the one or more morphological features are attributable to one or more unique groups of pixels in the image data.

169. The method of claim 164, wherein the generating the cell morphology map using image data comprises processing image data using a machine learning algorithm to group the images of the plurality of single cells into the plurality of morphologically-distinct clusters.

170. The method of claim 169, further comprising: extracting, using the machine learning algorithm, one or more morphological features of the image data from each cell of the plurality of single cells.

171. The method of claim 164, further comprising: annotating each morphologically-distinct cluster of the morphologically-distinct clusters to generate a plurality of annotated cell images belonging to each cluster of the morphologically-distinct clusters.

172. The method of claim 164, wherein the using the classifier to automatically classify the cellular image sample is performed on (i) a known population of cells in the cellular image sample, (ii) an unknown population of cells in the cellular image sample, or both (i) and (ii).

173. The method of claim 164, wherein the one or more of the clusters comprise sub-clusters.

174. The method of claim 164, wherein two or more of the clusters overlap.

175. The method of claim 164, further comprising: curating, verifying, editing, annotating, or a combination thereof, using an interactive annotation tool, the morphologically-distinct clusters.

176. The method of claim 175, further comprising: excluding, using the interactive annotation tool, one or more cells of the plurality of single cells that are incorrectly clustered.

177. The method of claim 175, further comprising: excluding, using the interactive annotation tool, debris or one or more cell clumps from the plurality of morphologically-distinct clusters.

178. The method of claim 175, further comprising: assigning weights, using the interactive annotation tool, to one or more clusters of the plurality of morphologically-distinct clusters.

179. A non-transitory computer-readable medium storing instructions that, when executed by a processor, perform a method comprising:
generating a cell morphology map comprising a plurality of morphologically-distinct clusters corresponding to different types or states of the cells using image data comprising tag-free images of a plurality of single cells; and
automatically classifying, by a classifier trained using the cell morphology map, a cellular image sample using (i) a correlation, (ii) a commonality, or both (i) and (ii), with one or more of the morphologically-distinct clusters.

180. The computer-readable medium of claim 179, wherein the generating the cell morphology map using image data comprises processing image data using a machine learning algorithm to group the images of the plurality of single cells into the plurality of morphologically-distinct clusters, wherein the method further comprises extracting, by the machine learning algorithm, one or more morphological features of the image data from each cell of the plurality of single cells.

181. The computer-readable medium of claim 179, wherein the method further comprises annotating each morphologically-distinct cluster of the morphologically-distinct clusters to generate a plurality of annotated cell images belonging to each cluster of the morphologically-distinct clusters.

182. A system, comprising:
at least one memory storing instructions; and
at least one processor to execute the instructions to perform operations comprising:
generating a cell morphology map using image data comprising tag-free images of a plurality of single cells, wherein the cell morphology map comprises a plurality of morphologically-distinct clusters corresponding to different types and/or states of the cells; and
automatically classifying, by a classifier trained using the cell morphology map, a cellular image sample using (i) a proximity, (ii) a commonality, or both (i) and (ii), with one or more of the plurality of morphologically-distinct clusters.

183. The system of claim 182, wherein the generating the cell morphology map using image data comprises processing image data using a machine learning algorithm to group the images of the plurality of single cells into the plurality of morphologically-distinct clusters, wherein the operations further comprise extracting, by the machine learning algorithm, one or more morphological features of the image data from each cell of the plurality of single cells.

184. The system of claim 182, wherein the automatically classifying is performed at least partially with a computer-implemented cloud-based system.
